(19) United States
(12) Patent Application Publication
(10) Pub. No.: US 2025/0188402 A1
(43) Pub. Date: Jun. 12, 2025

(54) SYSTEMS AND METHODS FOR ANALYZING AND MANAGING CELL CULTURE PROCESSES

(71) Applicant: Cellino Biotech, Inc., Cambridge, MA (US)

(72) Inventors: Matthias Wagner, Cambridge, MA (US); Ozge Whiting, Cambridge, MA (US); Deniz Aksel, Cambridge, MA (US); August Lin, Cambridge, MA (US)

(21) Appl. No.: 18/972,162

(22) Filed: Dec. 6, 2024

Related U.S. Application Data
(60) Provisional application No. 63/690,991, filed on Sep. 5, 2024.
(60) Provisional application No. 63/648,443, filed on May 16, 2024.
(60) Provisional application No. 63/553,380, filed on Feb. 14, 2024.
(60) Provisional application No. 63/606,929, filed on Dec. 6, 2023.

(51) Int. Cl. C12M 1/36; G06T 7/00

(52) U.S. Cl. CPC C12M 41/48; G06T 7/0016; G06T 2207/20081; G06T 2207/30024; G06T 2207/30072; G06T 2207/30241

(57) ABSTRACT

Methods, systems, and computer program products are provided for monitoring a cell culture, utilizing a predictive model to improve cell culture processes, measuring cell dynamics of a cell culture, managing cell clusters in a cell culture system, and training a machine learning model for managing cell clusters in a cell culture system. In various embodiments, a cell culture may be maintained in a cell culture chamber. The cell culture may comprise one or more cell colonies. One or more properties of the cell culture may be measured. One or more perturbations may be applied to all or a portion of the cell culture. The one or more properties of the cell culture may be measured after applying the one or more perturbations. The one or more properties may be compared before and after the one or more perturbations. The cell culture may be managed based on the comparison.

### CROSS-REFERENCE TO RELATED APPLICATIONS

This application claims the benefit of priority to U.S. Provisional Application No. 63/606,929, filed Dec. 6, 2023, U.S. Provisional Application No. 63/553,380, filed Feb. 14, 2024, U.S. Provisional Application No. 63/648,443, filed May 16, 2024, and U.S. Provisional Application No. 63/690,991, filed Sep. 5, 2024, the contents of each of which are incorporated herein by reference in their entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Cell therapies hold the promise of aiding millions around the world by treating a wide variety of ailments and illnesses. However, to treat patients cell therapy providers must manufacture cells numbering in the hundreds of millions to billions for each patient. Traditionally, cell manufacturing has been conducted manually, by technicians working in labs around the clock for months to generate a sufficient number of cells (that pass quality controls) for just one patient. This method of cell manufacturing is prohibitively expensive and slow, and thus becomes a bottleneck for cell therapy companies to overcome.

Efforts have been made to automate and/or scale up cell manufacturing, but these efforts are still in their infancy. There are many technical challenges that must be overcome. For example, it would be advantageous to shrink the footprint needed for manufacturing cells, or manufacture cells for multiple patients in the same space rather than requiring different clean rooms for each patient. However, this presents the challenge of preventing contamination of patient samples, particularly if the same equipment is being used on multiple patients. In another example, it would be advantageous to replace the manual work done by technicians (e.g., cell observation, cell picking, passaging, media changes) with automated workflows. However, the challenge becomes developing tools and software that at least match the quality of work done by technicians without damaging the cells during cell culture processes. In another example, the design of a cell culture container that can be used in automated systems and workflows is itself an engineering challenge. Thus there is a need in the art to solve many technical problems that stand in the way of robust, large scale, automated cell manufacturing. The systems and methods disclosed herein address a number of these technical problems.

### BRIEF SUMMARY

In various embodiments, a method of managing a cell culture process is provided. The method comprises: maintaining a cell culture in a cell culture chamber, the cell culture undergoing a cell culture process; measuring one or more properties of the cell culture; applying one or more perturbations to all or a portion of the cell culture; measuring the one or more properties of the cell culture after applying the one or more perturbations; comparing the one or more properties before and after the one or more perturbations; and managing the cell culture process based on the comparison.

Managing the cell culture process may comprise predicting a future state of the cell culture based on the one or more properties before and after the one or more perturbations and the comparison. Measuring the one or more properties may comprise acquiring images of the cell culture. The images may comprise time-series images. At least one of the one or more perturbations may be applied spatially. At least one of the one or more perturbations may be applied temporally. The cell culture chamber may comprise a closed fluidic cell culture chamber. The cell culture may be adhered to a first surface of the closed fluidic cell culture chamber. The first surface may be transparent and may enable optical imaging and optical cell removal of the cell culture. Measuring the one or more properties may comprise generating one or more cell maps. Comparing the one or more properties may comprise generating one or more differential maps. Managing the cell culture may comprise at least one of selectively removing one or more cells from the cell culture using a cell removal tool, applying one or more additional perturbations to the cell culture, harvesting the all or a portion of the cell culture for downstream processing, or terminating the cell culture process. The one or more perturbations may be applied to a test portion of the cell culture spatially separated from the rest of the cell culture.

The method may further comprise capturing images of the cell culture at a plurality of timepoints throughout the process, with the plurality of timepoints including an initial timepoint before application of the one or more perturbations and a final timepoint after application of the one or more perturbations, generating a map of the cell culture at the final timepoint, the map identifying one or more regions of the culture having a target cell type, back-tracing the one or more regions to earlier timepoints, based on this back-tracing, determining a first perturbation in the one or more perturbations that results in the one or more regions having the target cell type, and associating the first perturbation with the target cell type in a predictive model. The cell culture process may comprise one of reprogramming, expansion, differentiation, or rejuvenation. The map may be a spatial trajectory map, an -omic trajectory map, or a combination of both. The method may be iteratively repeated to improve the predictive model. The method may also comprise applying the predictive model to a future cell culture process.

A computer program product may be provided for managing a cell culture process. The computer program product may comprise a computer-readable storage medium having program instructions embodied therewith. The program instructions may be executable by a processor to cause the processor to perform a method according to any aspects of the described method of managing a cell culture process.

An imaging system may be provided for analysis and monitoring of a cell culture. The system may comprise a camera configured to capture at least one digital image of the cell culture. The system may also comprise a processor configured to receive the at least one digital image from the camera and to perform a method according to any aspects of the described method of managing a cell culture process.

In various embodiments, a method of utilizing a predictive model to improve cell culture processes is provided. The method comprises: performing a cell culture process on the cell culture, capturing images of the cell culture at a plurality of timepoints throughout the cell culture process, the plurality of timepoints including an initial timepoint, one or more intermediate timepoints, and a final timepoint, generating a map of the cell culture at the final timepoint, the map identifying one or more regions of the culture having a target cell type, back-tracing the one or more regions to earlier timepoints, based on this back-tracing, determining one or more initial or intermediate conditions of the cell culture at the initial timepoint that results in the one or more regions having the target cell type, and associating the one or more initial or intermediate conditions with the target cell type in the predictive model.

The cell culture process may comprise one of reprogramming, expansion, differentiation, or rejuvenation. The map may be a spatial trajectory map, an -omic trajectory map, or a combination of both. The one or more initial conditions may comprise spatial configurations of one or more cells in the cell culture. The method may be iteratively repeated to improve the predictive model. The method may also comprise applying the predictive model to a future cell culture process. Applying the predictive model may comprise at least one of: adjusting the spatial configuration of a cell culture at the beginning of the future cell culture process; adjusting the spatial configuration of the cell culture during the future cell culture process; and adjusting one or more parameters of global conditions applied during the future cell culture process. Adjusting the spatial configuration may comprise at least one of selective cell removal or selective extracellular matrix removal. A cell removal tool may be used to adjust the spatial configuration.

A computer program product may be provided for utilizing a predictive model to improve cell culture processes. The product may comprise a computer-readable storage medium having program instructions embodied therewith. The program instructions may be executable by a processor to cause the processor to perform a method according to any aspects of the described method of utilizing a predictive model to improve cell culture processes.

An imaging system may be provided for utilizing a predictive model to improve cell culture processes. The system may comprise a camera configured to capture at least one digital image of the cell culture. The system may also comprise a processor configured to receive the at least one digital image from the camera and to perform a method according to any aspects of the described method of utilizing a predictive model to improve cell culture processes.

In various embodiments, a method of measuring cell dynamics of a cell culture is provided. The method comprises: capturing images of the cell culture at a first timepoint and a second timepoint; generating spatial maps of the cell culture at the first timepoint and the second timepoint; determining an optimal transport plan for the cell culture based on the spatial maps, wherein the optimal transport plan represents cell dynamics of the cell culture between the first timepoint and the second timepoint; and estimating one or more cell dynamics of the cell culture based on the optimal transport plan.

The one or more cell dynamics may comprise cell motion, cell division, cell death, and cell state changes. The method may further comprise altering a cell culture process performed on the cell culture based on the estimated cell dynamics. The alteration to the cell culture process may be performed using a spatially-selective cell removal tool. The cell removal tool may comprise an optical coating that interacts with a pulsed laser to locally remove cells.

A computer program product may be provided for measuring cell dynamics of a cell culture. The product may comprise a computer-readable storage medium having program instructions embodied therewith. The program instructions may be executable by a processor to cause the processor to perform a method according to any aspects of the described method of measuring cell dynamics of a cell culture.

An imaging system may be provided for measuring cell dynamics of a cell culture. The system may comprise a camera configured to capture at least one digital image of the cell culture. The system may also comprise a processor configured to receive the at least one digital image from the camera and to perform a method according to any aspects of the described method of measuring cell dynamics of a cell culture.

In various embodiments, a method of managing cell clusters in a cell culture system is provided. The method comprises: maintaining a cell culture in a cell culture container, the cell culture comprising one or more cell clusters; collecting observation data on a first cell cluster in the one or more cell clusters; determining, using a cell culture management agent, a cell operation action to perform on the first cell culture based on the observation data, wherein the cell culture management agent utilizes reinforcement learning to make the determination; and performing the cell operation action using a cell removal tool.

The cell operation action may comprise at least one of removing the first cell cluster, removing a region of the first cell cluster, shepherding the first cell cluster along a surface of the cell culture container, splitting the first cell cluster into a plurality of sub-clusters, or keeping the first cell cluster as-is. The observation data may comprise data obtained from time-series images of the first cell culture. The cell culture management agent may be trained using at least one of in-silico or in-vitro cell culture experiments. The in-silico cell culture experiments may be managed by a cell culture simulation engine. The cell culture management agent may generate a cell removal map that defines the cell operation action.

A computer program product may be provided for managing cell clusters in a cell culture system. The product may comprise a computer-readable storage medium having program instructions embodied therewith. The program instructions may be executable by a processor to cause the processor to perform a method according to any aspects of the described method of managing cell clusters in a cell culture system.

A system may be provided for managing cell clusters in a cell culture system. The system may comprise a data store configured to store observation data. The system may also comprise a processor configured to receive the observation data from the data store and to perform a method according to any aspects of the described method of managing cell clusters in a cell culture system.

In various embodiments, a method for managing cell clusters or regions in a cell culture system is provided. The method comprises: reading image data of at least one cell cluster or region in a cell culture at a plurality of time steps, applying a pretrained machine learning model to the image data at each of the plurality of time steps, and determining, from the trained machine learning model, instruction data defining a cell removal operation for the cell culture system.

The method may further comprise instructing the cell culture system to execute the instruction data. The method may further comprise receiving additional data of the cell cluster or region. The additional data may include at least one of nutrient data, metabolic data, pH data, O2 data, and temperature data. The method may further comprise providing instruction data from a prior time step to the pretrained machine learning model. The method may further comprise synchronizing the additional data to the image data. Applying the pretrained machine learning model may comprise extracting one or more features from the image data for the present and each preceding time step. Extracting the one or more features may comprise providing the image data for the present and each preceding time step to a feature extraction network. Applying the pretrained machine learning model may comprise providing the image data for the present and each preceding time step to the pretrained machine learning model. The pretrained machine learning model may comprise an artificial neural network. The instruction data may comprise a cell removal action. The instruction data may comprise a cell removal map. The cell removal map may be selected from a plurality of cell removal templates. Applying the pretrained machine learning model to the image data may comprise applying one or more label maps to the image data.

A computer program product may be provided for managing cell clusters or regions in a cell culture system. The product may comprise a computer-readable storage medium having program instructions embodied therewith. The program instructions may be executable by a processor to cause the processor to perform a method according to any aspects of the described method of managing cell clusters in a cell culture system.

A system may be provided for managing cell clusters or regions in a cell culture system. The system may comprise a data store configured to store observation data. The system may also comprise a processor configured to receive the observation data from the data store and to perform a method according to any aspects of the described method of managing cell clusters in a cell culture system.

In various embodiments, a method of training a machine learning model for managing cell clusters in a cell culture system is provided. The method comprises generating an initial training set, the initial training set comprising a plurality of action-outcome pairs, each action selected from a set of cell removal actions, wherein generating the initial training set comprises simulating one or more cell experiments; determining a reward value for each of the action-outcome pairs; and training a machine learning model based on the action-outcome pairs and associated reward values.

The method may also comprise providing the trained machine learning model for updating based on one or more additional experiments. The pretrained machine learning model may have been pretrained according to the method outlined above. A computer program product for managing cell clusters in a cell culture system may comprise a computer-readable storage medium having program instructions embodied therewith. The program instructions may be executable by a processor to cause the processor to perform a method according to any aspects of the described method of training a machine learning model for managing cell clusters in a cell culture system.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative implementations, in which the principles of the disclosure are utilized, and the accompanying drawings of which:

FIG. 1 is a block diagram of a cell culture system in accordance with various implementations;

FIG. 2 is a flow chart of a method of operating a cell culture system in accordance with various implementations;

FIGS. 3A-E are diagrams illustrating global perturbation and observation of a cell culture in accordance with various implementations;

FIGS. 4A-F are diagrams illustrating localized spatial perturbation and observation of a cell culture in accordance with various implementations;

FIGS. 5A-G are diagrams illustrating spatial perturbation and observation of a test portion of a cell culture in accordance with various implementations;

FIG. 6 is a block diagram illustrating a method of measuring properties of a cell culture by perturbation in accordance with various implementations;

FIG. 7 is a diagram of a prior art approach to managing a cell culture process;

FIG. 8 is a diagram of another prior art approach to managing a cell culture process;

FIG. 9 is a diagram of a multi-step prior art approach to managing a cell culture process;

FIG. 10 is a diagram of another multi-step prior art approach to managing a cell culture process;

FIG. 11 is a diagram of another multi-step prior art approach to managing a cell culture process;

FIGS. 12A-F are diagrams illustrating the alteration of -omic trajectories of cells in accordance with various implementations;

FIGS. 13A-E are diagrams illustrating the use of spatial and -omic relationships to manage a cell culture process in accordance with various implementations;

FIG. 14 is a diagram illustrating training a predictive model with spatial maps in accordance with various implementations;

FIG. 15 is a diagram illustrating management of a cell culture process using a predictive model and selective cell removal in accordance with various implementations;

FIG. 16 is a diagram illustrating observation of the initial spatial configuration of a cell culture process in accordance with various implementations;

FIG. 17 is a diagram illustrating management of a cell culture process using a predictive model trained on initial spatial configurations in accordance with various implementations;

FIG. 18 is a diagram illustrating methods for tracing cell trajectories of a cell culture in space in accordance with various implementations;

FIGS. 19A-B are diagrams illustrating parallel back-tracing of cell trajectories in both the spatial and -omic domains in accordance with various implementations;

FIGS. 20A-C are diagrams illustrating methods for mapping -omic and spatial features of a cell culture process into a single latent space to train predictive models in accordance with various implementations;

FIG. 21 is a diagram of a differentiation cell culture process prior to optimization by a predictive model in accordance with various implementations;

FIG. 22 is a diagram of a differentiation cell culture process after optimization by a predictive model in accordance with various implementations;

FIG. 23 is a flow chart of a method of training and using a predictive model based on cell culture process trajectories in accordance with various implementations;

FIG. 24 are images showing an example of fluorescence tracking and selective cell removal of a multi-day cell culture process in accordance with various implementations;

FIG. 25 are images showing an example of colonialization of a homogeneously mixed population of fluorescent and non-fluorescent iPSCs in accordance with various implementations;

FIGS. 26A-D are diagrams illustrating a cell population at four different timepoints in accordance with various implementations;

FIGS. 26E-G illustrate cell dynamics maps generated from images of the cell populations at the timepoints illustrated in FIGS. 26A-D in accordance with various implementations;

FIGS. 26H-I illustrate examples of aggregate cell dynamics for the cell population of FIGS. 26A-D that may be generated from the transport maps shown in FIGS. 26E-G in accordance with various implementations;

FIGS. 27A-B are cell dynamics graphs for comparing observed cell dynamics to baseline cell dynamics behavior in accordance with various implementations;

FIGS. 28A-C are diagrams illustrating splitting of test colonies from a primary cell colony in accordance with various implementations;

FIGS. 29A-C are diagrams illustrating the use of multiple scales of density measurement used to classify cell colony regions in accordance with various implementations;

FIG. 30 is a diagram illustrating the reconstruction of a cell transport plan using data from two timepoints in accordance with various implementations;

FIG. 31 is a diagram illustrating the use of transport plans to trace cellular origins and/or destinations in a cell culture process in accordance with various implementations;

FIGS. 32A-C are diagrams illustrating the calculation of an optimal transport plan for a cell culture while restricting cells to a single type/state in accordance with various implementations;

FIGS. 33A-C are diagrams illustrating a solution for the optimal transport challenge depicted in FIGS. 32A-C in accordance with various implementations;

FIG. 34 is a block diagram of a cell culture management system in accordance with various implementations;

FIG. 35 is a block diagram of a cell culture simulation engine in accordance with various implementations;

FIG. 36 is a block diagram illustrating a training framework for a reinforcement learning approach to cell culture management in accordance with various implementations;

FIG. 37 is a block diagram illustrating a training method for a reinforcement learning model in accordance with various implementations;

FIG. 38 is a block diagram of a cell culture management system using an exemplary agent architecture in accordance with various implementations;

FIG. 39 illustrates an example action space for a cell culture management agent in accordance with various implementations;

FIG. 40 is a block diagram of an example neural network used in a cell culture management system in accordance with various implementations;

FIG. 41 illustrates a method for cell culture management in accordance with various implementations;

FIGS. 42A-G illustrate machine learning generated (ML-generated) cell removal maps and subsequent removal and proliferate steps to expand a cell colony in accordance with various implementations;

FIG. 43 is a diagram of a cell culture workflow for detecting anomalies in a cell culture process in accordance with various implementations;

FIG. 44 is a method for measuring and managing a cell culture in accordance with various implementations; and

FIG. 45 is an exemplary computing node in accordance with various implementations.

These and other features of the present implementations will be understood better by reading the following detailed description, taken together with the figures herein described. The accompanying drawings are not intended to be drawn to scale. For purposes of clarity, not every component may be labeled in every drawing.

### DETAILED DESCRIPTION

Disclosed herein are systems and methods including an automated or semi-automated cell culture system that may quickly and accurately produce output cell products and that is easily scalable to enable large scale biological manufacturing. The system may include cell imaging subsystems to acquire images of a cell culture, a cell removal tool to selectively remove one or more cells during the cell culture process, a computing subsystem that controls the cell removal tool based on the acquired images, or any combination thereof. The computing subsystem may apply machine learning to data collected by the system (e.g., imaging data, sensor data, input, and output assay data) to determine how to effectively manage the cell culture to reach the desired output. This allows for dynamic monitoring and control of how the cell culture develops from input cells to output cell products. The automated or semi-automated nature of the system removes the need for manual human intervention at many stages of cell culture development, thus reducing the time and cost of making output cell products. It also allows for easy scalability, as the computing subsystem may monitor and control multiple cell culture processes at the same time.

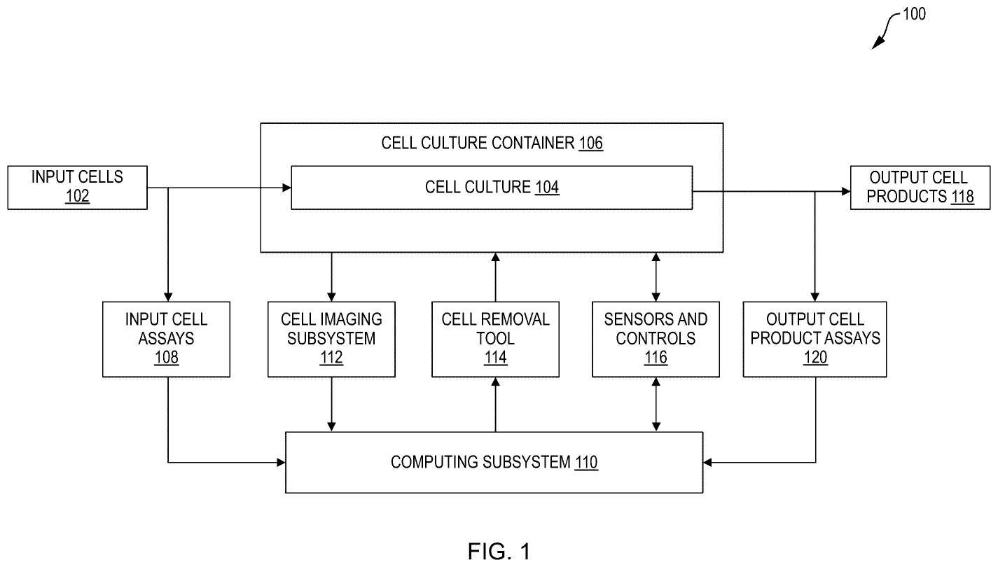

FIG. 1 is a block diagram of a cell culture system 100 in accordance with various implementations. The cell culture system 100 receives input cells 102 as “source” cells upon which the cell culture system 100 performs various cell culture processes. The input cells 102 may be sorted, expanded, or otherwise modified prior to the cell culture performed by the cell culture system 100. Input cell types may include, but are not limited to, somatic cells (including but not limited to fibroblasts, mature blood and progenitor cells, such as CD34+ cells and erythroblasts, keratinocytes, epithelial cells, including blood and urine-derived epithelial cells, Sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, other muscle cells, adipocytes, mesenchymal stem cells, and generally any live somatic cells. The term “somatic cells,” as used herein, also includes adult stem cells and pluripotent stem cells (including but not limited to induced pluripotent stem cells and embryonic stem cells).

The input cells 102 may be analyzed with one or more input cell assays 108 which serve to quantify the state of the input cells 102. The input cell assays 108 may be nondestructive (such as cell counting) or a sample may be extracted for tests including, but not limited to, genomic profiling, gene expression assays such as PCR, qPCR, microarray, single-cell RNA sequencing, whole exome sequencing (WES), whole genome sequencing (WGS), karyotyping, short tandem repeat (STR) analysis, DNA methylation assays such as BS-Seq, RRBS or MeDIP-Seq, histone modification assays such as ChiP-Seq or CUT&RUN, chromatin accessibility assays such as ATAC-Seq, DNase-Seq, or FAIRE-Seq, telomere assays by qPCR, terminal restriction fragment analysis, FISH, STELA, TeSLA, or SMTLA, sterility testing (testing for bacteria and viruses), or other phenotype analysis including but not limited to cell surface antigen or intracellular staining-based immunofluorescence or flow analysis, and cell viability, morphology and migration assays, or any other implementations known to persons of ordinary skill in the art. The sample extraction can be performed using automated or semi-automated processes within a closed cell culture environment to enable continued propagation of the cell culture within a sterile environment. The results of these assays are transmitted to a computing subsystem 110, which may use the results in various software applications to monitor, predict, and control the cell culture process performed by the cell culture system 100.

The input cells 102 are placed into a cell culture 104, where they will remain for the duration of the processes performed by the cell culture system 100. The cell culture 104 may reside in a cell culture container 106. The cell culture container 106 may include one or more chambers to hold the cell cultures, and may take the form of microwell plates, flasks, stackable cell culture containers, closed cassette systems, microfluidic chambers, purpose-built bioreactor vessels, roller bottles, roll-to-roll adherent cell culture systems, or any other implementations known to persons of ordinary skill in the art. The cell culture container 106 may be a closed/sealed sterile environment for the cell culture 104 and fluid media used in cell culture processes.

The cell culture 104 may be used for a number of cell processes performed and monitored by the cell culture system 100, including but not limited to: cell reprogramming (into pluripotent or multipotent forms), cell differentiation, cell trans-differentiation, cell expansion, cell sorting or purification, clonal isolation, cell gene editing, cell-based protein production, cell-based viral production, cell rejuvenation or regeneration, combinations thereof, or any other implementations known to persons of ordinary skill in the art.

The cell culture container 106 may be in a format that allows for observation of the cell culture 104 at regular intervals using an imaging subsystem 112. For example, the cell culture container 106 may include a closed cassette system having at least one transparent or semi-transparent surface that allows for light or laser-based imaging and editing. The imaging subsystem 112 may be configured to provide label-free imaging suitable for long-term cell culture observation, although some implementations may include fluorescent imaging capability for immunofluorescent or other labeled images. Label-free modalities employed by the imaging subsystem 112 may include, but are not limited to, brightfield imaging, phase imaging, darkfield imaging, transmission imaging, reflection imaging, quantitative phase imaging, holographic imaging, two-photon imaging, autofluorescence imaging, Fourier ptychographic imaging, defocus imaging or any other implementations known to persons of ordinary skill in the art. The imaging subsystem 112 may be shared between one or more of the cell culture containers 106.

The cell culture system 100 further includes a cell removal tool 114 for selectively removing cells or affecting changes to cells within the cell culture 104. The cell removal tool 114 may selectively remove cells in the cell culture 104 at a regional, colony-specific, and/or cell-specific level. The cell removal tool 114 may be configured for selective destruction and/or removal of cells or cell regions, and non-destructive operations on cells (including intracellular delivery of compounds into cells or extraction of compounds from cells). The cell removal tool 114 may interact with the cell culture 104 through a variety of directed energy mechanisms. In other words, the cell removal tool 114 may generate energy that is directly used to remove cells and/or converts energy of one form (e.g., light, mechanical) into energy of another form to achieve cell removal. The mechanism by which the cell removal tool 114 acts upon cells in the cell culture may include, but not be limited to, robotic systems that mechanically actuate a tip or tool across the cell culture, magnetic actuators in conjunction with magnetic tools that interact with the cell culture, systems that are configured to selectively apply an electric field across portions of the cell culture, ultrasound systems that are configured to apply ultrasonic energy to portions of the cell culture, droplet or particle ejection/acceleration systems that are designed to impact droplets or particles on portions of the cell culture, optical systems that are designed to deliver optical energy to portions of the cell culture, combinations thereof, or any other implementations known to persons of ordinary skill in the art. The cell removal tool 114 may be shared between one or more of the cell culture containers 106.

Optical mechanisms for cell removal may include, but are not limited to, optical systems that direct energy directly into cells or surrounding media in the cell culture, optical systems that direct energy into particles or dyes that are added to the cell culture media (including but not limited to particles functionalized in a manner to attach to specific cells, or that are taken up by cells), or optical systems that direct energy into particles or films that are on surfaces proximate to portions of the cell culture, or any other implementations known to persons of ordinary skill in the art. Optical mechanisms may operate on the cell culture by a number of approaches including, but not limited to, elevating the local temperature to a point where cells are destroyed due to heat damage, elevating local temperature to cause boiling and/or bubble formation to cause portions of the cell culture to detach from a surface, or elevating local temperature rapidly in order to cause rapid bubble formation and then subsequent collapse to affect mechanical forces on the local cell membranes, or combinations thereof.

The cell culture system 100 may also include a number of sensors and controls 116 which may measure or act upon the cell culture 104. For example, the sensors and controls 116 may carry out functions such as measuring media conditions within the cell culture 104, causing fresh media to be supplied, or adding reagents or gases to adjust media conditions for optimal cell culture growth. Sensors that sense the state of the cell culture 104, cell culture media, and/or surrounding cell culture container 106 may include, but are not limited to, temperature sensors, humidity sensors, gas composition sensors including but not limited to O₂ and CO₂ concentration sensors, gas flow rate sensors, dissolved gas sensors including but not limited to dissolved O₂ sensors, liquid flow rate sensors, and sensors to measure cell culture media constituents (such as nutrients, waste products, vitamins, metabolites, proteins, extracellular vesicles, cell mass, or cell debris) including but not limited to optical absorption sensors, optical scattering sensors, mass spectroscopic sensor systems, optical or electrical pH sensors, and viscosity sensors.

Controls that may interact with the cell culture 104 or the cell culture container 106 may include, but are not limited to, liquid handling systems that inject or extract various liquids to/from the cell culture 104 or the cell culture container 106, environmental control systems that control the temperature or other environmental parameters of the cell culture 104 or the cell culture container 106, power systems that provide electrical power to the cell culture container 106, and mechanical or robotic systems that may move or manipulate the cell culture container 106 or portions thereof.

The computing subsystem 110 may be configured to control the other components of the cell culture system 100 to perform the specified cell culture process on the cell culture 104 to produce output cell products 118. The output cell products 118 may include both cells and cell-derived products, and may be harvested from the cell culture 104. Output cell products 118 that may be produced by the computing subsystem 110 may include, but are not limited to, induced pluripotent stem cells, differentiated cells of various cell types (e.g., retinal pigment epithelial (RPE) cells, heart cells, lung cells, neural cells, brown adipose tissue cells), proteins (e.g., cytokines, antibodies, hormones), lipid particles (e.g., exosomes), viral particles, somatic cells (including but not limited to fibroblasts, mature blood and progenitor cells, such as CD34+ cells and erythroblasts, keratinocytes, epithelial cells, including blood and urine-derived epithelial cells, Sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, other muscle cells, adipocytes, mesenchymal stem cells, generally any live somatic cells, and the combination of any of the above. The term “somatic cells,” as used herein, also includes adult stem cells.

The output cell products 118 may be measured by output cell product assays 120 in order to determine critical product parameters such as phenotype distribution, protein production, gene activation, genomic makeup (including but not limited to genomic profiling assays such as PCR, qPCR, microarray, single-cell RNA sequencing, whole exome sequencing (WES), whole genome sequencing (WGS), karyotyping, short tandem repeat (STR) analysis, DNA methylation assays such as BS-Seq, RRBS or MeDIP-Seq, histone modification assays such as ChiP-Seq or CUT&RUN, chromatin accessibility assays such as ATAC-Seq, DNase-Seq, or FAIRE-Seq, telomere assays by qPCR, terminal restriction fragment analysis, FISH, STELA, TeSLA, or SMTLA, sterility testing (testing for bacteria and viruses)), or other phenotype analysis including but not limited to cell surface antigen or intracellular staining and immunofluorescence or flow analysis and cell viability, morphology and migration assays, or potency assays such as self-renewal and teratoma formation assays, and germ-layer differentiation assays. The output assay data may be conveyed to the computing subsystem 110 in order to refine predictive models (based on image data, sensor data, information from prior cell culture processes, and other information sources) for cell culture monitoring and control. Output cell product assays 120 may include, but not be limited to, viability assays, cell counting, flow cytometry, immunostained imaging assays, PCR assays (including but not limited qPCR, ddPCR), RNA sequencing assays including single-cell RNA assays, cell differentiation assays, embryoid body formation assays, trilineage differentiation assays, karyotyping assays, DNA sequencing, or any other implementations known to persons of ordinary skill in the art.

The computing subsystem 110 is configured to gather data from a range of sources, organizes the data in a manner that allows it to make predictions of success/quality/functionality of the cell culture 104, and in many cases do so on a cell-by-cell, colony-by-colony, or region-by-region basis. For example, using local cell density and proliferation rate data obtained through analysis of the time series of label-free images provided by the imaging subsystem 112, in conjunction with data regarding the input cells (in order to control for patient-specific factors, for instance), and optionally information from sensors 116, and based on a large number of observed histories and corresponding cell quality data measured by the output cell product assays 120, the computing subsystem 110 may predict which regions of cells are most likely to yield superior cell products, and which regions are less likely to yield good product. In situations where cell media is limited or there is competition between cells for space in the cell culture container 106, the computing subsystem 110 may instruct the cell removal tool 114 to remove the regions or even individual cells predicted to underperform.

Another function of the computing subsystem 110 is to use cell data derived from imaging in conjunction with environmental parameters and sensor data from the sensors and controls 116 and assay data from the input cells 102 and/or the output cell products 118 in order to pre-emptively adjust cell culture conditions according to cell count, proliferation rate, differentiation status, phenotype, or other factors in addition to real-time cell media readings. Using a model trained on previous iterations, the computing subsystem 110 may adjust media conditions such as fresh media feed, media type, temperature, pH, dissolved Oxygen levels, reagent or vitamin levels or other global cell culture properties using the controls 116. Similarly, the computing subsystem 110 may use cell data obtained from imaging, potentially in conjunction with cell media sensor data, to determine when the cell culture 104 is ready for harvest. Actuators utilized by the controls 116 may include, but are not limited to: liquid handling robots, liquid circulation systems including valves and pumps, temperature control elements, pH controllers, gas exchange mechanisms to control dissolved gases or any other implementations known to persons of ordinary skill in the art.

The computing subsystem 110 may control the cell removal tool 114 to manage the cell culture 104 according to cell management algorithms (for example, to maintain a certain cell density, to maintain certain exclusion areas within the cell culture container), in a timed manner (for example, delivering gene-activating or gene-editing compounds to cells at a specific interval), and/or as a result of predictions made by the computing subsystem 110 (for example, removal of cells predicted not to yield the desired phenotype or optimal level of function). Cell culture management may include both destruction of cells and/or colonies (including inducing apoptosis, lysing, physically removing) as well as selective delivery of compounds into cells and/or regions of cells via intracellular delivery mechanisms, or selective extraction of compounds from the cells via intracellular delivery mechanisms, or management of confluence or density of a cell culture over time (e.g., for at least 10 days), or other types of cell manipulation and management.

The computing system 110 may include elements that perform conventional image processing (including but not limited to filtering, normalization, contrast enhancement, z-stack processing, thresholding, histogram transformations, edge detection, correlations, convolutions, frequency space operations, blob detection, morphological operations, registration, warping, object detection, object tracking or combinations thereof), deep learning based image processing (including but not limited to convolutional neural networks, fully-connected neural networks, semantic and instance-level segmentation, encoder-decoder networks, multi-scale algorithms, recurrent networks, visual attention models, vision transformers, generative adversarial models, U-Nets, ResU-Net, SegNet, X-Net, ENet, BoxENet, long short-term memory neural networks, and combinations thereof), statistical models, pattern recognition, statistical learning (including but not limited to linear regression, non-linear regression, hierarchical regression, generalized linear models, logistic regression, log-linear models, non-parametric models), machine learning (including but not limited to decision trees, random forest, support vector machines, neural nets, deep learning, association models, sequence modeling, genetic modeling), reinforcement learning, clustering techniques including hierarchical and non-hierarchical clustering, supervised machine learning models, unsupervised machine learning models, databases (including but not limited to SQL databases and NoSQL databases), visualization tools for image, cell, colony, clone and other data, combinations of these elements, or any other implementations known to persons of ordinary skill in the art.

The computing subsystem 110 may also include data storage for storing image data, sensor data, the results of data analysis, and program code that the computing subsystem 110 executes. The computing subsystem 110 may also include input/output devices to allow users to view data and monitor and control the cell culture system 100, or to transfer data in and out of the cell culture system 100. For example, the computing subsystem 110 may include display screens, monitors, communications/interface ports, keyboards, audio systems, and the like. The computing subsystem 110 may be proximate to the other components in the cell culture system 100 (e.g., a local computer) or may be remote from the other components in the cell culture system 100 (e.g., a cloud server). In some implementations, the computing subsystem 110 may have one or more components proximate the other components in the cell culture system 100 and some components remote from the other components in the cell culture system 100. The computing subsystem 110 may be configured to communicate with the other components in the cell culture system 100 utilizing a wired and/or wireless connection (e.g., Ethernet cables, optical fiber, Wi-Fi, Bluetooth), and may be configured to communicate with external components utilizing a wired and/or wireless connection. The computing subsystem 110 may have additional functionality and components not disclosed herein, but would be apparent to a person of ordinary skill in the art.

The cell culture system 100 may be configured to allow extended cell culture processes to be performed within a single cell culture container 106 using the cell removal tool 114. Because the cell removal tool 114, as directed by the computing subsystem 110, can selectively remove cells from cell culture, the cell culture does not overgrow the cell culture container, and therefore does not require frequent transfers (“passaging”) which are stressful on cell populations, disrupt cell processes, introduce potential sterility and contamination issues, and make time series tracking of cell-, region-, colony- or clone-specific behavior impossible. Thus, the combination of continuous monitoring via image and sensor data—enabled by the single-container process—may allow the computing subsystem 110 to predict the optimal regions or cells to remove to maintain low enough cell density to remain in the single cell culture container 106. In the process the cell culture system 100 may also perform in-place “sorting” of cells to enrich the population according to real-time measurements.

The cell culture system 100 may be configured to monitor and control multiple cell culture processes at the same time, and on multiple patient samples at the same time. For example, a first set of cell culture containers may contain cells for a first patient. The first set of cell culture containers may undergo a cell expansion process. A second set of cell culture containers may contain cells for a second patient, and those cells may undergo a cell differentiation process. The first and second set of cell culture containers may be located in the same environment, but each cell culture container may be sealed such that there is no cross-contamination between patient samples. The cell culture system 100 may apply a first machine learning model optimized for cell expansion to the first set of cell culture containers as those cells undergo cell expansion, and may at the same time apply a second machine learning model optimized for cell differentiation to the second set of cell culture containers as those cells undergo cell differentiation. In general, the cell culture system 100 may be configured to parallel process many cell samples from many patients, and apply different machine learning models to each cell sample based on the intended cell culture process and other factors. The cell culture system 100 may be configured to monitor and control multiple parallel cell culture processes for long periods of time, for example over the course of at least 10 days or 30 days.

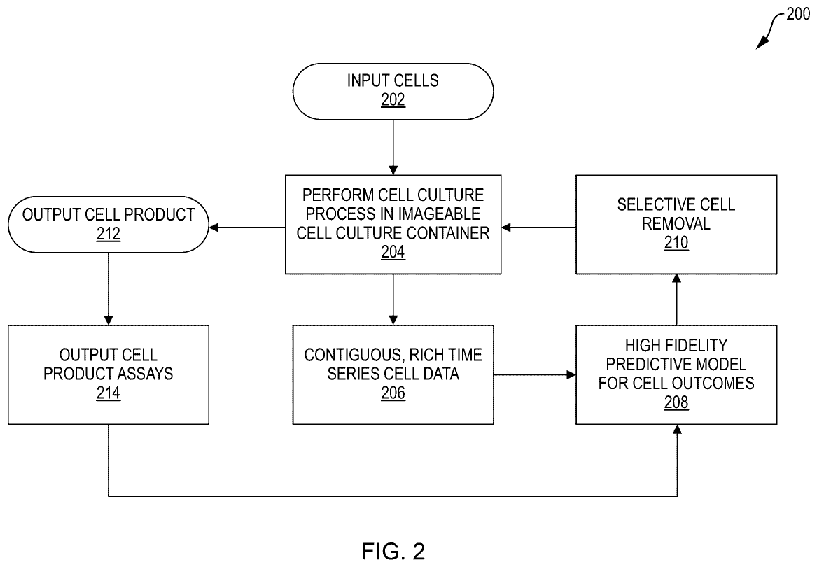

FIG. 2 is a flow chart of an example method 200 of operating a cell culture system in accordance with various implementations. The method 200 may be performed by a cell culture system, such as cell culture system 100. In block 202, input cells are seeded into a cell culture container that is fully imageable and able to support a cell culture for the duration of the cell process. This results in a single-container, fully-imageable cell culture. The cell culture container may provide a closed, sterile environment for cell culture processes. In block 204, a cell culture process may be performed on the single-container, fully-imageable cell culture. The cell culture process may be sustained within a single container for the duration of the process (as opposed to transferring, sometimes selectively, cells from container to container to maintain property density). The cell culture process may be monitored and controlled by a computing subsystem in the cell culture system. However, despite this single-container extended culture capability, transfers of cells from container to container, or container to multiple containers, is not precluded.

In block 206, the cells may be observed with an imaging subsystem to acquire unbroken, contiguous, rich time series of cell data. In block 208, the computing subsystem may analyze the cell data to develop a high fidelity predictive model for cell outcomes. The computing subsystem may utilize the predictive model to adjust the cell culture process dynamically. For example, in block 210, the computing subsystem may control a cell removal tool to selectively remove cells from the cell culture to manage the cell culture process (e.g., de-densify the cell culture over time). The selective removal, in turn, is optimally configured to improve the predicted yield, functionality, phenotype, or other properties of the output cell product. The method 200 may iterate through the steps of collecting imaging data, refining the predictive model, and editing the cell culture until the output cell product is produced in block 212.

In block 214, output cell product assay 214 may be performed on the output cell product at the end of a cell culture operation. The results of the assays may be used in conjunction with the time series cell data to adjust the predictive model in block 208. In some cases, the output cell product may be harvested dynamically from the process (for example, a subset of cells may be selected and removed from the cell culture, or cell products within the media are removed from the cell culture) and the corresponding assay results immediately fed back into the predictive model. In this manner, the method 200 allows for a completely automated method for dynamically processing and managing cell cultures, from input cells to output cell products. This allows for faster, more accurate cell culture processes without the time and expense of manual human intervention, which in turn reduces the time and cost for producing output cell products. This approach is also easily scalable to enable large scale biological manufacturing.

### Cell Culture Measurements Via Modulation

For various biomanufacturing and regenerative medicine applications (e.g., production of cells for cell and gene therapies), there are many sources of variability in cell manufacturing and cell culture processes. These sources of variability include, but are not limited to, source cell phenotype and state (including donor- and clone-dependent genomic and epigenetic state), process inputs (e.g., media, reagents, extracellular matrix deposition) and conditions, stochastic intracellular processes, stochastic multi-cell processes such as clumping and clustering, and associated chemical and mechanical processes with high variability (for example, cell passaging).

There has been much effort to exert better control over cell culture processes to reduce this variability and increase uniformity, yield, and quality of manufactured cells. In addition, there is a desire in the art to characterize cells, cell regions, colonies, or entire cell cultures to estimate quality, purity, and/or progression during a cell culture process, even in noisy, variable environments. Characterization is preferably performed using only label-free and/or non-invasive approaches to minimize the effect on cells, but which generally offers lower signal fidelity than labeled (e.g., fluorescently-labeled) or invasive techniques that physically sample cell material from a bioreactor or cell culture container.

One approach to making cell measurements in this noisy, constrained environment is integration or averaging of signals over long time periods to improve the signal-to-noise ratio (SNR) of estimates. Another approach is spatial averaging over regions of cells, entire cell colonies, or over entire cell populations within a cell culture container (for example, measuring cell confluence within a container to assess proliferation rates). However, averaging can lead to significant lags in detection of events or changes in the cell culture, and/or the loss of information related to spatial localization of such events.

Thus there is a need in the art to achieve higher-SNR discrimination of cell states from the available measurement techniques for monitoring and control of cell culture processes. Better estimates from such measurements would allow for better dynamic control of bioprocesses, both at the global level (e.g., adjustment of environmental conditions, adjustment of media and reagents, timing of steps/phases of the process, early disqualification of batches, etc.) and at the local level through spatially-selective cell modification or removal.

The systems and methods disclosed herein include a system for introducing perturbations into a bioprocess being performed on a cell culture while making time series observations. The system may use these perturbations in conjunction with label-free observations of an entire cell culture, one or more cell colonies, regions of cells, or individual cells by their dynamics under perturbation. The system may further estimate properties of cells, colonies, regions, or entire cell cultures based on these dynamical observations. The system may further control the bioprocess based on the estimated properties, by adjusting the global conditions of the bioprocess, or by selectively modifying or removing cells based on spatially-resolved properties. For example, a cell culture system has disclosed herein may utilize perturbations to monitor and control cell culture processes to improve yield and output cell quality.

Further implementations disclosed herein include a method of monitoring a cell culture, the method including maintaining a cell culture in a cell culture chamber (the cell culture comprising one or more cell colonies) measuring one or more properties of the cell culture, applying one or more perturbations to all or a portion of the cell culture, measuring the one or more properties of the cell culture after applying the one or more perturbations, comparing the one or more properties before and after the one or more perturbations, and managing the cell culture based on the comparison.

While a cell culture system (e.g., cell culture system 100) is usually configured to keep cell cultures in a steady state and allow cells to proliferate (optionally, in a clonal manner), it may be beneficial to change the conditions in all or a portion of the cell culture to measure modulated responses from the cell culture. The modulated responses may be indicative of various properties of the cell culture, and the cell culture system may utilize this information to modify the cell culture process to improve yield and/or quality of the cell culture, or to make decisions about when to end, abort, or change phases or conditions in a cell culture process.

The cell culture system may initiate these modulated responses by perturbing one or more parameters in all or a portion of the cell culture. The term “perturbation” or “modulation” is hereinafter used to describe spatial or temporal changes to a cell culture environment intended to produce a response from a cell culture that may be measured. A perturbation may be a temporal condition modulation, in which the perturbation is applied to the cell culture as a function of time and effects are measured as a function of time. A perturbation may also be spatial in nature, in which a perturbation is applied to a designated region of the cell culture. In some implementations, a perturbation may be both spatial and temporal in nature.

One example of a temporal perturbation is impulse condition modulation, in which a single “impulse” or a short change in a cell culture environment parameter introduces a temporary change in cell culture itself. For example, the impulse condition may be a short-term introduction of a dissociation enzyme into the bioprocess. The cell culture may include one or more “test” sub-colonies of cell colonies under observation that may be prone to detachment from the cell culture surface for some cell statuses, and not prone to detachment for others. The dissociation enzyme may cause detachment of some of the test sub-colonies, which may be harvested for testing or in which detachment themselves may be indicative of certain cell culture conditions.

Another example of a temporal perturbation is step condition modulation, in which a step (or discrete increase) in a cell culture parameter is imposed at a time point in the bioprocess to elucidate differentials in cell, cell colony, or cell region responses. For example, the step may be a change in media additives at pre-set time point in a cell culture process. The cell culture system may then observe changes of cell dynamics across the cell culture to map out regions that are more and less likely to have reached the target point of the cell culture process (e.g., process may be disqualified if too many laggard cells are observed, or laggard regions may be selectively removed via a cell removal tool in order to purify/better synchronize the process).

Another example of a temporal perturbation is a waveform condition modulation, in which a repeated modulation of a parameter is applied to a cell culture process at a certain frequency. This cyclical change of conditions may alter cell characteristics or dynamics in a way that may be observed by the cell culture system as a way of characterizing the cells. For example, a fluidic cassette enclosing the cell culture process may be alternately incubated in a normoxic (20-21% O₂) and physiological/hypoxic (2-3% O₂) environment on a 48-hour cycle. Changes in proliferation rate per cell colony corresponding to the modulation frequency may be extracted via label-free imaging and cell density mapping. Changes in magnitude, direction, and phase lag of cell proliferation rate for each cell colony may be recorded as a marker for cell colony status that may be used in a predictive model to rank cell colonies by functionality and viability.

An example of a spatial perturbation is media spatial modulation, in which different media compositions may be added sequentially into a cell culture container (e.g., fluidic chamber or closed cassette system) in which fluid flow is non-mixing. This may cause regions in the cell culture container with different media conditions and components, and thus the cells may respond differently based on which region they are in. Additionally, gradients within the media over the cell culture may be set up, or result from diffusion. Cell features and dynamics in the different zones, or in the gradient region, may subsequently be measured.

Another example of a spatial perturbation is extracellular matrix (ECM) patterning modulation, in which ECM may be spatially patterned across the cell culture to cause differential cell behaviors such as propagation (or lack thereof), proliferation, motility, differentiation, etc. These patterns may be applied in the vicinity of multiple cell populations/colonies to record differentials in dynamics that may be correlated with cell functionality and state.

Another example of a spatial perturbation is cell patterning modulation, in which cell sheets, colonies, clusters, or individual cells may be patterned in various spatial arrangements to look at dynamic cell behavior, such as motility, differentiation, proliferation, apoptosis, etc. For example, test cell colonies may be physically separated from main cell colonies, and the ability of test cell colonies to re-group through motility/colony re-joining behavior may be observed via time-series imaging.

The examples described above are non-limiting examples of perturbations that may be applied to a cell culture process. In general, perturbations are changes in one or more cell culture environment parameters applied spatially, temporally, or a combination of both. Many kinds of parameters may be changed in a perturbation, and a non-limiting list of parameters is provided herein.

One example of a parameter that may be perturbed is cell patterning by cell removal. Adherent cells may be patterned using a cell removal tool to establish patterns that elicit characteristic behaviors, such as proliferation, changes in morphology, differentiation, motility, and cell or colony regrouping behaviors. Examples include patterning “test cells” (e.g., not main colonies or populations, but cells extracted from these populations as described herein) in arrangements that are prone to differentiation or apoptosis under certain conditions.

Another example of a parameter that may be perturbed is ECM patterning to control where cells may grow. This parameter may be combined with cell patterning to isolate cells or examine how cells grow through specific ECM patterns and/or densities. Another example of a parameter that may be perturbed is ECM detachment, in which ECM under the observed cells may be partially or completely removed/detached from the cell culture surface, for example by optical means, or by opto-mechanical means such as laser-initiated microbubbles.

Another example of a parameter that may be perturbed is surface texturing and/or modulus, in which cells may be moved to regions with specific surface textures or elastic moduli for the purpose of modulating cell function. For example, a set of cells may be moved to an area with parallel striations to assess alignment with the striations.

Another example of a parameter that may be perturbed is temperature, which may affect different cell types differently. Temperature may be modulated globally or locally (e.g., using illumination by wavelengths that are absorbed by the cell media or cell culture container to locally heat a region of the cell culture surface). Another example of a parameter that may be perturbed is pH of the fluid media, which may be changed directly or indirectly through control of CO₂ gas levels.

Another example of a parameter that may be perturbed is light, and particularly illumination of cells with one or more wavelengths of light. This may be done spatially and/or temporally. If spatially selective, the light may target “test cells” derived from the main cell population. For example, cells may be illuminated by light to induce cell signaling within some cells and induce behaviors depending on cell state. In other examples, light may locally modify cell media to induce effects on local cells. In other examples, light may interact with coatings on the cell culture surface, including but not limited to ECMs or optical films, that in turn cause modulation of cell behavior.

Another example of a parameter that may be perturbed is pressure. Hydrostatic pressure may be varied in closed fluidic chambers to differentially affect cells of different states or types. Another example of a parameter that may be perturbed is stretching or other mechanical strain effects on cell cultures. This may include stretching of the surface that cells are adhered to, application of force by flow, application of force by centrifugation, application of force by liquid-gas interfaces that are translated across the cell surface, or application of force by microbubbles, which may be initiated by means of ultrasonic, laser bubble nucleation, or other means.

Another example of a parameter that may be perturbed is the application of electric fields to the cell culture, including fields that cause electroporation of the cell membrane to various degrees. In some examples, an electric field may provide electrical stimulation to cells of particular states/phenotypes (and not others). For example, an electric field may stimulate contraction or other behaviors of specific cells to provide contrast with non-contracting cells, or cells that contract out of synch.

Another example of a parameter that may be perturbed are dissolved gasses (for example, setting up a hypoxic environment) in the cell media, including but not limited to oxygen and carbon dioxide. For example, physiological (2-3%) versus atmospheric oxygen may cause differentiated proliferation rates and other effects which may be observed in morphology or growth dynamics, depending on cell state and phenotype.

Another example of a parameter that may be perturbed is cell media protocol. The cell media protocol may be changed in various ways. For example, the media change schedule may be changed (e.g., by changing the frequency of changes). Changing the media may have multiple effects on the cell culture resulting from fluid flow, replenishment of nutrients, and depletion of waste products. The fluid media components may also be changed. The media components that may be changed includes, but are not limited to, levels of nutrients, proteins, growth factors, cytokines, hormones, vitamins, caffeine, small molecules meant to promote cell differentiation, rho-associated protein kinase (ROCK) inhibitors that might affect cell proliferation, reprogramming or differentiation factors, and other components that may affect cell motility, morphology, cell-cell adhesion, or cell-surface adhesion. Additional media components that may be changed, added, or removed from the fluid media may include extracellular matrix components that may affect cell structure, antibodies that may activate specific cellular pathways, nanoparticles or beads (potentially coated), and compounds that differentially affect cell health/survival (e.g., quercetin may be added as a differential stressor for pluripotent cells).

Another media component that may be changed is osmolarity, Na+, Ca++, and other ionic concentrations. Varying ionic concentrations in the fluid media may affect cell membranes. Osmotic pressure on the cell membrane may modulate cell behavior (including proliferation) depending on the cell state/phenotype. Additionally, osmotic pressure modulation may allow imaging of cells in two states (e.g., low pressure, high pressure), with each state and the differential giving information about individual cell or cell neighborhood state.

Another media component that may be perturbed are flow speeds/rates. Fluid media flows over cells, like other stimuli, may have modulating effects on gene regulatory networks. In some instances it is known to foster differentiation into specific cell types. The use of media flow in conjunction with observation of cells may indicate a range of cell states and characteristics. This may be combined with patterning of cells, including use of “test cells” or “test colonies” that are patterned in a manner to be particularly sensitive to flow conditions.

Another example of a parameter that may be perturbed are antibiotics, which may be added to stress specific cell populations differentials. For example, antibiotics may be added when a removal antibiotic-resistant gene cassette has been added as part of a gene editing process. In this case, imaging observations may detect cells, regions, or colonies that have an effect induced by the antibiotic prior to others, which may be used to drive proactive removal of cells based on these measurements. Such modulation and detection may minimize the amount of antibiotic required, and reduce stress on desirable cells.

Another example of a parameter that may be perturbed is the introduction of additional cells or co-culture (including new cell types) into the cell culture, which may be used to modulate the existing cell functions or to observe interactions between the cell types. The introduced cells may be removed from the cell culture container after the observation is made via washing, change of media conditions, and/or by use of a cell removal tool. Another example of a parameter that may be perturbed is the introduction of extracellular matrix or hydrogels, or other materials into the cell culture that coat cell layers and modulate cell functions. Another example of a parameter that may be perturbed is cell synchronization. Small molecules or other cues may be used to synchronize cell cycles, and such synchronization may work for some cells/phenotypes better than others.

Synchronization may be used as the observed signal itself (global synchronization, or local/regional degree of synchronization), or it may be used in conjunction with other modulated factors to more accurately measure their effects. A description of synchronization in iPSC cultures is described in Yiangou, Loukia, et al., “Method to Synchronize Cell Cyle of Human Pluripotent Stem Cells without Affecting Their Fundamental Characteristics,” Stem Cell Reports 2019, 12(1):165-179, which is hereby incorporated by reference in its entirety.

In some implementations, perturbations may be used in combination, either sequentially or simultaneously, and mixing both temporal and spatial perturbations. The effects of multiple applied perturbations may be de-multiplexed in computational analysis of cell observations. In cases in which “test cells” are separated spatially from the main colonies/cell populations and exposed to the modulating parameters, these “test cells” may be removed from the cell culture after the modulated measurements are made (for example, when such isolated test cells are potentially damaged by the modulated conditions).

The perturbations disclosed herein may be measured by various measurement modalities and techniques to monitor the cell culture and observe the effects of the perturbations. The measurement techniques contemplated herein include various imaging modalities disclosed herein, but may also include other measurement and imaging techniques that include but are not limited to transmission imaging (including but not limited to brightfield, darkfield, Zernicke phase, differential interference contrast (DIC), quantitative phase imaging, lens-less imaging, holographic interference imaging, ptychographic imaging, etc.), fluorescence imaging (including but not limited to imaging with fluorescent labels or endogenously-expressed fluorescent molecules such as those that may be added as part of a gene editing workflow), spectral imaging/mapping (including but not limited to Raman spectroscopy, surface-enhanced Raman spectroscopy, near infrared (NIR) spectroscopy, mid-infrared spectroscopy, optoacoustic spectroscopy, photothermal imaging, and other spectroscopy techniques that measures the state of cell media by its spectral features), cell surface imaging (including but not limited to surface plasmon resonance (SPR) and total internal reflectance microscopy), spent media analysis (i.e., analysis of media that has been used in the cell culture to look at nutrient, waste, and cell (by)product levels), or combinations of any of the above.

Measurement of perturbations may provide information about various cell or cell culture properties and dynamics in the cell culture. One example of properties that may be measured from perturbations is cell morphology, which may include but is not limited to refractive index, size, count, and shape of cell body, nucleus, nucleoli, projections, and other cellular components. Cell morphology may also include refractive index differentials between media, cell body, nucleus, nucleoli, and other intracellular components. Another example of properties that may be measured from perturbations is cell absorption (including but not limited to pigmentation levels in the cells). Another example of properties that may be measured from perturbations is colony morphology, which includes but is not limited to area, shape factors, perimeter morphology, density distribution, holes, appearance of 3-dimensional areas, etc.

Another example of properties that may be measured from perturbations is cell motility and direction, including but not limited to movement of individual cells on the cell culture surface, movement of colony borders, and velocity of movement as well as direction. Cell motility may also include re-grouping behaviors and cells or colonies moving to join with one another or larger colonies. Another example of properties that may be measured from perturbations is proliferation rate and direction (e.g., local cell division rate, and directionality of growth, if any). Another example of properties that may be measured from perturbations is cell/colony survival (e.g., survival of cell colonies, sometimes patterned in specific sizes or shapes to make them susceptible to modulating conditions). Another example of properties that may be measured from perturbations are cell/colony differentiation parameters, such as rate of differentiation of cells or colonies, and location of differentiated cells (along edges of colony, for example).

Another example of properties that may be measured from perturbations is 3D colony morphology (e.g., 3D properties of cell colonies including height, morphology, distribution of 3D areas). Another example of properties that may be measured from perturbations is apoptosis (e.g., level and spatial distribution of apoptotic cells in a colony, region, etc.). Another example of properties that may be measured from perturbations is local media usage (e.g., local nutrient consumption, waste production, pH change, metabolite concentrations—all as a result of cell activity).

Another example of properties that may be measured from perturbations are cell-surface adhesion properties, such as the level of adhesion of cells or colonies to the cell culture surface. Adhesion properties may measured by either direct imaging of focal adhesions or contact of cells with the cell culture surface, or indirectly through observation of detachment of cells, regions, or colonies from the cell culture surface. Another example of properties that may be measured from perturbations are cell-cell junctions, which includes morphology and strength of cell-cell adhesions as visualized by imaging, or measured in other manners such as flow-throughs resulting from pressure gradients, electrical resistance measurements, or other means. Combinations of these properties may be measured, and absolute levels as well as changes/differentials resulting from the perturbed parameters may be recorded by the cell culture system. Properties may be mapped spatially over the cell culture container (e.g., a cell map generated by the cell culture system), or measured as an average for the cells in a cell culture container.

The cell and cell culture properties that may be measured due to perturbations applied to a cell culture may be used to predict or estimate various cell characteristics via models that have been pre-trained using in-process data together with cell assay data. The properties of the cells, regions, colonies or entire populations that may be derived, predicted, or estimated from the tracked data (including the data derived from the condition modulations) includes but is not limited to phenotype, genotype, pluripotency, differentiation capacity to specific lineages and cell types, differentiation state, cellular aging, functionality, viability, viral load, protein production, exosome production, expansion capacity, and engraftment potential. This information may be mapped at the cellular, regional, colony or whole population level of the cell culture.

The cell culture system, and particularly its computing subsystem, may utilize the cellular properties derived from measurements of perturbations of the cell culture in a variety of manners to further manage the cell culture. For example, the cell culture system may determine whether the cell culture process is deviating from its intended course and not likely to yield good cells, and terminate the cell culture process if it has deviated beyond a threshold amount. In another example, the cell culture system may utilize the information about cell and cell culture properties to determine whether to move to a new phase of the cell culture process. For example, the cell culture system may determine that a cell culture has completed a certain phase of the cell culture process and is ready to transition to the next phase, either in the same cell culture container or in a new cell culture container.

In another example, the cell culture system may utilize the information about cell and cell culture properties to control fluid media or other conditions in the cell culture container. This may include active control of media, gas, temperature, or other conditions to keep the cell culture within a desired band. In some implementations, the modulated conditions may be the same as the controlled conditions, with the modulation being small deviations on top of the slowly-varying baseline condition in the cell culture.

In another example, the cell culture system may utilize the information about cell and cell culture properties to perform selective removal of cells from the cell culture. For example, the cell culture system may use a cell removal tool to selectively remove cells that are deemed, via the perturbations and subsequent observation, to not meet desired characteristics, to purify the cell culture, and/or to make room for the selective expansion of cells or colonies deemed “good.”

In another example, the cell culture system may utilize the information about cell and cell culture properties to selectively harvest cells. For example, the cell culture system may use a cell removal tool to selectively harvest a portion of cells or cell material, either for downstream use, or for further characterization by assays based on the information obtained from observing the perturbed cell culture. In another example, the cell culture system may utilize the information about cell and cell culture properties to predict downstream functionality or yield of the cell culture. For example, the cell culture system may be configured to predict differentiation potential into a specific cell line or germ layer, or predicting engraftment and function in vivo, from observing the perturbed cell culture.

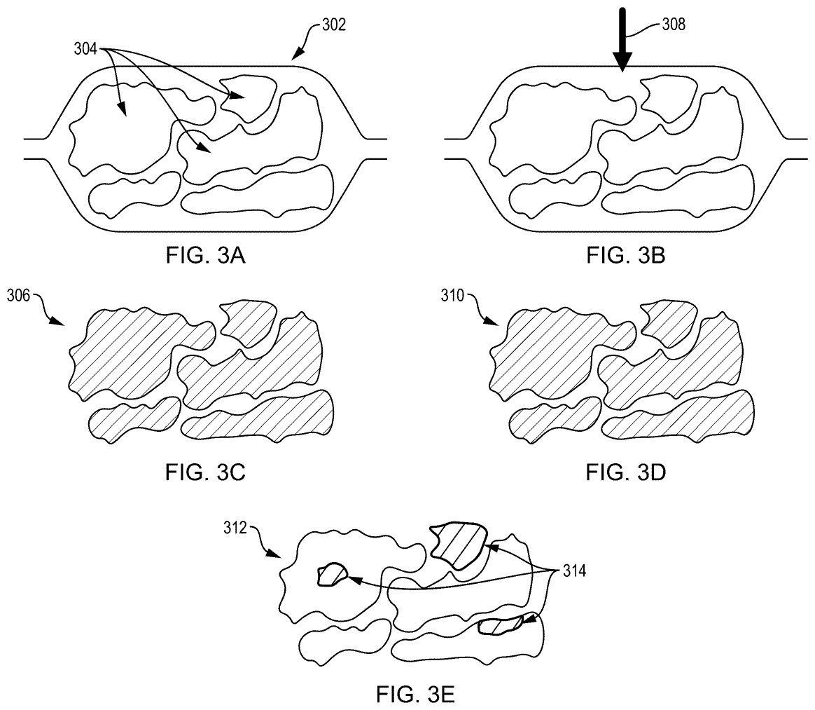

FIGS. 3A-E are diagrams illustrating global perturbation and observation of a cell culture in accordance with various implementations. FIG. 3A illustrates a fluidic cell culture chamber 302, which may be part of a cell culture container (e.g., a closed cassette system). The cell culture chamber may be monitored and controlled by a cell culture system, which includes one or more imaging subsystems, cell removal tools, computing subsystems, and other supporting components. An adherent cell culture 304 is growing on one or more surfaces of the cell culture chamber 302, and the cell culture 304 is undergoing a cell culture process (e.g., reprogramming, expansion, differentiation).

FIG. 3B illustrates the application of a perturbation 308 into the cell culture chamber 302 that alters the environment for the cell culture 304. The perturbation 308 may change any one or more parameters as described herein and may be a spatial or temporal perturbation, or a mixture of both. The perturbation 308 may be global such that it is applied to the entire cell culture 304. For example, the perturbation 308 may be a change in oxygen concentration, change in temperature, and/or a change in media constituents.

FIG. 3C illustrates a cell map 306 that is created via imaging of the cell culture 304. The cell culture system may have a cell imaging subsystem that is configured to capture images of the cell culture 304 over time, using any of the modalities described herein. A computing subsystem in the cell culture system may be configured to convert the images into the cell map 306. The cell map 306 may be multidimensional to capture multiple properties of the cell culture 304 as described herein. The cell map 306 may be generated using conventional image processing, or a range of machine learning, deep learning, and artificial intelligence models that convert an as-measured image into maps of predicted cell properties. In some implementations, more than one cell map 306 may be generated. For example, the cell map(s) 306 may include cell density maps, phenotype maps, local cell division rate maps, and motility maps that are generated from time points in a time series of images. Such a series of images would capture cell properties and dynamics under a first cell culture condition.

FIG. 3D illustrates a cell map 310 that is created via imaging of the cell culture 304 after the perturbation 308 is applied to the cell culture chamber 302. The cell map(s) 310 may be one or more maps that are composed from time series measurements of the cell culture 304 to measure the local dynamics of the cell culture 304 under the new conditions created by the perturbation 308.

FIG. 3E illustrates a differential map 312 of the cell culture 304. The differential map 312 may be generated as the difference between cell maps 306 and 310, and may indicate differences of certain cell properties or cell dynamics between the cell map 306 before the perturbation 308 and the cell map 310 after the perturbation 308. The cell culture system may generate multiple differential maps 312, each measuring the differential of a particular cell property or cell dynamic. The differential map(s) 312 may indicate regions 314 with specific alterations or non-alterations to their properties or dynamics. For example, certain regions of cells may divide faster or slower than others as a result of the perturbation 308. In another example, cell morphology may change differentially under different media conditions.

Based on the differential map(s) 312, the cell culture system may evaluate the overall quality or developmental phase of the cell culture 304 and manage on the cell culture 304 based on this information (e.g., moving to a different phase of the cell culture process, aborting the process, removing certain cells, dividing or translating certain cell colonies). For example, a cell removal tool may be used to remove regions of cells that are not properly differentiating according to the differential map 312, so as to clear space for the proliferation of properly-differentiating cells.

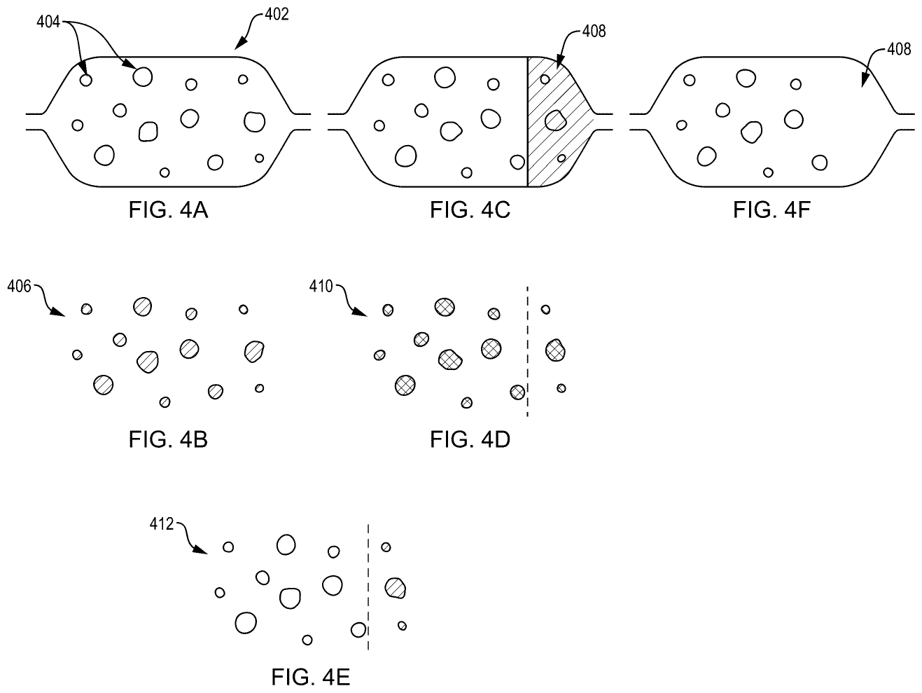

FIGS. 4A-F are diagrams illustrating localized spatial perturbation and observation of a cell culture in accordance with various implementations. FIG. 4A illustrates a fluidic cell culture chamber 402, which may be part of a cell culture container (e.g., a closed cassette system). The cell culture chamber may be monitored and controlled by a cell culture system, which includes one or more imaging subsystems, cell removal tools, computing subsystems, and other supporting components. The cell culture chamber 402 may include two or more ports for exchanging fluid cell media. A plurality of cell colonies 404 is growing on one or more surfaces of the cell culture chamber 402, and the cell colonies 404 are undergoing a cell culture process (e.g., reprogramming, expansion, differentiation).

FIG. 4B illustrates a cell map 406 that is created via imaging of the cell colonies 404. The cell culture system may have a cell imaging subsystem that is configured to capture images of the cell colonies 404 over time, using any of the modalities described herein. A computing subsystem in the cell culture system may be configured to convert the images into the cell map 406. The cell map 406 may be multidimensional to capture multiple properties of the cell colonies 404 as described herein. The cell map 406 may be generated using conventional image processing, or a range of machine learning, deep learning, and artificial intelligence models that convert an as-measured image into maps of predicted cell properties. In some implementations, more than one cell map 406 may be generated. For example, the cell map(s) 406 may include cell density maps, phenotype maps, local cell division rate maps, and motility maps that are generated from time points in a time series of images. Such a series of images would capture cell properties and dynamics under a first cell culture condition.

FIG. 4C illustrates a spatial region 408 of the cell culture chamber 402 that is subjected to a spatially-selective perturbation as described herein, and the parameter changed may include any one or more parameters as disclosed herein. For example, the region 408 may have a different media condition applied to modulate local cell and cell colony behavior. In some implementations, both the region 408 and the remaining region of the cell culture chamber 402 may each have different perturbations applied. In other implementations, the cell culture chamber 402 may be divided into multiple zones, each with a different perturbation applied (and in some cases, one region with no perturbation applied as a control). In a fluidic cell culture chamber, these zones may be achieved by slow exchanges that keep specific media boundaries intact. In the example in which the media condition is varied, the variation may include the addition or removal of specific growth factors or transcription factors, extracellular matrix, addition of antibiotics, or other additives. In other implementations, the media delivered to a particular zone may contain fluorescent labels, indicator dyes, or other components to elucidate cell state.

FIG. 4D illustrates a cell map 410 that is created via imaging of the cell colonies 404 after the spatially-selective perturbation is applied to the region 408 (the dotted line indicates the boundary of the region 408). The cell map(s) 410 may be one or more maps that are composed from time series measurements of the cell colonies 404 to measure the local dynamics of the cell colonies 404 under the new conditions created by the spatially-selective perturbation.

FIG. 4E illustrates a differential map 412 of the cell colonies 404. The differential map 412 may be generated as the difference between cell maps 406 and 410, and may indicate differences of certain cell properties or cell dynamics between the cell map 406 before the spatially-selective perturbation and the cell map 410 after the spatially-selective perturbation. The cell culture system may generate multiple differential maps 412, each measuring the differential of a particular cell property or cell dynamic. The differential map(s) 412 may indicate changes in cell colony characteristics or dynamics between the region 408 (the boundary indicated by the dotted line) and the rest of the cell colonies 404 due to the spatially-selective perturbation. These differences may be used by the cell culture system to further manage the cell culture and make decisions on maintaining or changing cell culture conditions, continuation of the cell culture, etc.

FIG. 4F illustrates an example of an action taken by the cell culture system after analysis of the differential map 412. In this example, the cell culture system may control a cell removal tool to remove the cell colonies in the region 408 affected by the spatially-selective perturbation. This operation may be performed when, for example, the perturbation is potentially harmful or destructive to the cells and so should be removed to not effect the cell culture as a whole. For example, if a perturbation was applied that causes cell colonies to differentiate into a cell type that is undesirable to measure their stability, then the cell colonies subject to the treatment should be removed after the measurements are complete. Likewise, if the spatial-selective perturbation includes UV or other illumination for the purpose of measuring cell stability or mutation rate, the targeted cells should be removed after the differential testing.

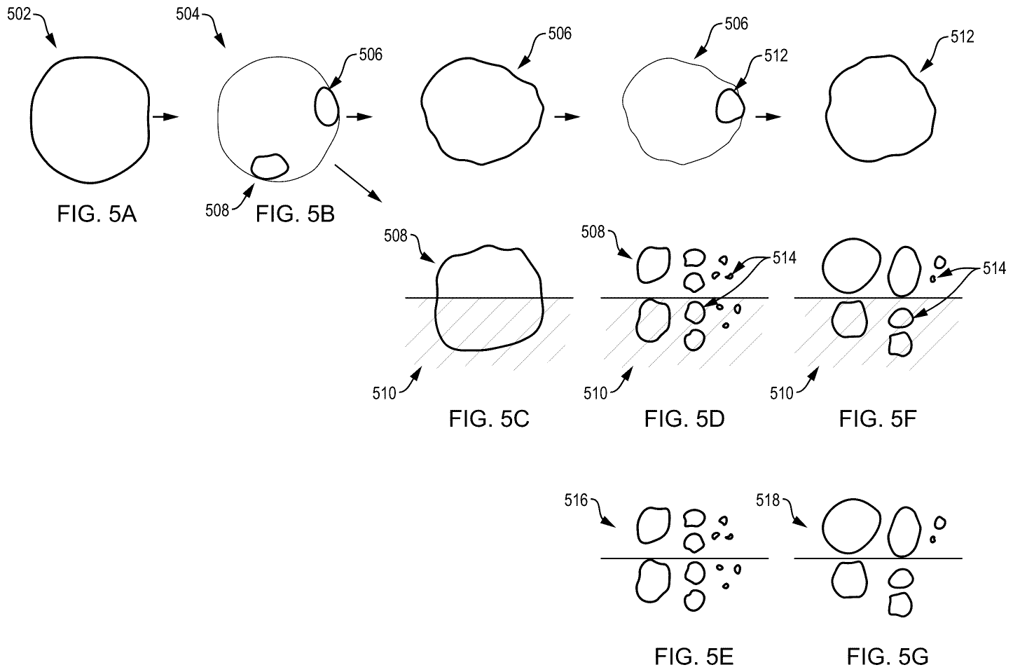

FIGS. 5A-G are diagrams illustrating spatial perturbation and observation of a test portion of a cell culture in accordance with various implementations. FIG. 5A illustrates a cell colony 502, which may be growing in a fluidic cell culture chamber. The cell culture chamber may be part of a cell culture container (e.g., a closed cassette system, well plate). The cell culture chamber may be monitored and controlled by a cell culture system, which includes one or more imaging subsystems, cell removal tools, computing subsystems, and other supporting components. The cell colony 502 may be adhered to one or more surfaces of the cell culture chamber.

FIG. 5B illustrates a cell removal operation on the cell colony 502 using a cell removal tool. The cell culture system may base the cell removal operation on cell colony images or cell maps derived from the images. The cell removal operation may remove most of the original cell colony 502 (shown by outline 504) and leave two sub-colonies: a main colony 506 and a test colony 508. The main colony 506 may be used to propagate the cell culture through a cell culture process (e.g., reprogramming, expansion, differentiation), while the test colony 508 may be used to test for various cell properties and dynamics by applying perturbations to it. As described herein, repeated use of the cell removal tool with proliferative cells may be used to manage their density, and also translate cell colonies across a growth surface.

FIG. 5C illustrates the cell culture at a later time point, in which the main colony 506 has expanded, and the test colony 508 has been translated and expanded. The test colony 508 may be translated across the growth surface using the cell removal tool as described herein. The test colony 508 may be at least partially translated into a region 510 that has spatially-varying characteristics (in other words, a localized spatial perturbation). For example, the perturbation may be the presence or amount of ECM adhered to the growth surface. In other examples, the perturbation may be a change in the growth surface texture. In other example, the perturbation may be changes in local flow rates. In the example shown in FIGS. 5A-G, the perturbation may be a change in ECM density.

FIG. 5D illustrates additional cell removal operations on the cell colonies using the cell removal tool. The main colony 506 may be reduced in size again to new main colony 512 (with the outline showing the original size of the main colony 506). This may be done, for example, to control the density and/or proliferation rate of the main colony 506. The test colony 508 may be split into multiple sub-colonies 514, which may vary in shape, size, distance from one another, or other properties. The sub-colonies 514 may be used to measure overall colony state based on the spatial perturbation in the region 510.

FIG. 5E illustrates a cell map 516 that is created via imaging of the sub-colonies 514. The cell culture system may have a cell imaging subsystem that is configured to capture images of the sub-colonies 514 over time, using any of the modalities described herein. A computing subsystem in the cell culture system may be configured to convert the images into the cell map 516. The cell map 516 may be multidimensional to capture multiple properties of the sub-colonies 514 as described herein. The cell map 516 may be generated using conventional image processing, or a range of machine learning, deep learning, and artificial intelligence models that convert an as-measured image into maps of predicted cell properties. In some implementations, more than one cell map 516 may be generated. For example, the cell map(s) 516 may include cell density maps, phenotype maps, local cell division rate maps, and motility maps that are generated from time points in a time series of images. The faded line indicates the boundary of the region 510.

FIG. 5F illustrates the cell culture at a later time point, in which the new main colony 512 has expanded, and some sub-colonies 514 have also expanded. A portion of the sub-colonies 514 located in the region 510 have been exposed to the localized spatial perturbation in the region 510 and thus may have different cell properties or dynamics than the sub-colonies 514 that have expanded outside of the region 510.

FIG. 5G illustrates a cell map 518 that is created via imaging of the sub-colonies 514 after a portion of them have been allowed to expand in the region 510 having a localized spatial perturbation. A differential map may be created from the cell maps 516, 518 to measure cell/colony behavior under a range of conditions, while maintaining the main colony 512 under ideal growth conditions. For example, the sub-colonies 514 may have a range of starting conditions (e.g., colony size, colony morphology, inter-colony distances, local colony conditions such as ECM) based on how they are divided by the cell removal tool. Applying the perturbation to this range of different sub-colonies may show characteristics including but not limited to survival (including under flow), proliferation rate (e.g., area, cell count), apoptosis rate, morphology changes, colony re-forming healing behavior, and directed or spontaneous differentiation. The resulting data from the sub-colonies 514 may be used to predict the state and/or quality of the main colony 512. After data has been collected, the test sub-colonies 514 may be removed using a cell removal tool.

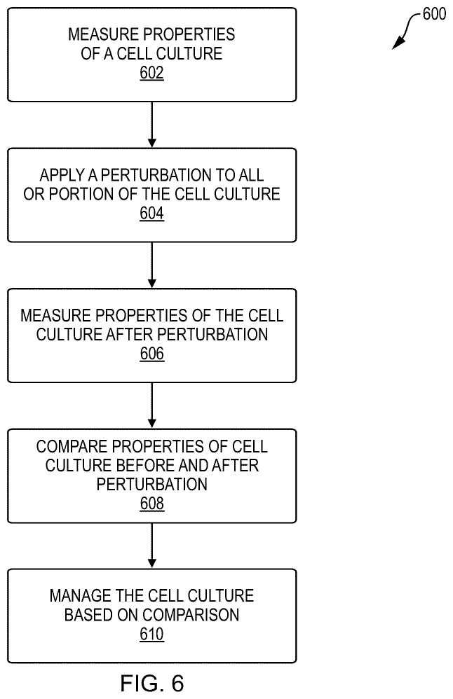

FIG. 6 is a block diagram illustrating a method 600 of measuring properties of a cell culture by perturbation in accordance with various implementations. The method 600 may be performed by a cell culture system as disclosed herein. The method 600 may be applied to a cell culture undergoing a cell culture process in a cell culture container. The cell culture may be, for example, an adherent cell culture (e.g., iPSCs). The cell culture container may be, for example, a closed cassette or multi-well plate having one or more closed fluidic cell culture chambers. The cell culture container may include a semi-transparent first surface upon which the cell culture is adhered, the first surface having an optical film configured to allow for optical imaging and optical cell operation actions.

In block 602, the cell culture system may measure properties of the cell culture. For example, the cell culture system may include a cell imaging subsystem that captures time-series images of the cell culture, and a computing subsystem may derive the properties of the cell culture from the time-series image. The cell culture system may generate one or more cell maps that represent various properties of the cell culture over the cell culture surface, as disclosed herein.

In block 604, the cell culture system may apply one or more perturbations to all or a portion of the cell culture. The perturbation may be a change in one or more parameters of the cell culture process as disclosed herein, and may be spatial, temporal, or a combination of both in nature. In some implementations, the cell culture system may spatially separate a portion of the cell culture as a test portion to apply the perturbations to. For example, the cell culture system may designate a region of the cell culture chamber as the test region, and any cells and cell colonies within that region become the test portion of the cell culture. In another example, the cell culture system may split off a portion of the cell culture (e.g., split a portion of a cell colony as described with reference to FIGS. 5A-G) and translate the portion to a different region of the cell culture chamber in which perturbations are applied.

In block 606, the cell culture system may measure properties of the cell culture after the perturbation is applied to the cell culture. The cell culture system may generate one or more cell maps that represent the properties of the cell culture over the cell culture surface, as disclosed herein.

In block 608, the cell culture system may compare properties of the cell culture before and after the perturbation. For example, the cell culture system may generate one or more differential maps that show changes in properties of the cell culture before and after the perturbation(s) are applied.

In block 610, the cell culture system may manage the cell culture based on the comparison. For example, the cell culture system may terminate the current cell culture process, move the cell culture process to a new phase, modify media or other environmental conditions of the cell culture, selectively remove cells from the cell culture, selectively harvest some of the cells, predict downstream functionality or yield, or any or management, monitoring and control functions disclosed herein. In this manner, the method 600 allows for dynamic testing and measurement of various cell culture properties which may be used to improve the management of cell culture processes, resulting in improved yield and quality and reducing the chance of failure.

### Systems and Methods for Measurement and Control of Cell Culture Process Trajectories

Biological processes, such as cell culture processes, may be conceptualized as a continuum of dynamic changes in the state of the cells. Cells undergoing a cell culture process may be characterized by a “trajectory,” or a path through a multi-dimensional expression space that traverses the various cellular states associated with a process, like differentiation. For example, transcriptomics is the study of all RNA molecules in a cell and thus transcriptomic trajectory is thus the trajectory of RNA in cells during a cell culture process. Transcriptomic trajectories may be an information-rich predictor of cell culture processes and functions of cell generation processes, such as expansion, reprogramming (including partial reprogramming), differentiation, and trans-differentiation. Besides transcriptomic trajectories, cell trajectories may also be constructed based on other cell parameters or characteristics, herein generally termed “-omics” trajectories. The term “-omics” or “-omic” as used herein is defined as cell characteristics, including but not limited to genomic, epigenetic, proteomic, and other states that characterize the cell phenotype, functionality, and viability (e.g., DNA sequences, epigenetic markers including but not limited to chromatin accessibility and methylation, karyotype, ploidy, RNA expression, and protein expression or composition).

Transcriptomic trajectory and spatial trajectory (the trajectory of spatial characteristics of cells during a cell culture process) are closely linked, as would be expected from developmental biology. This is true in both 2D and 3D cell culture processes, albeit less complex in two-dimensional space. Cell culture process trajectories and outcomes in terms of phenotype and functionality usually have distinctive, coherent spatial patterns (e.g., not random cell-to-cell variations). In addition, many cell culture process trajectories are highly sensitive to the initial and intermediate spatial conditions of the cell culture, at multiple scales.

In current cell culture processes, control of cell culture process trajectory is generally limited to global control of cell culture environment conditions. Often, observation of cell culture process trajectory is made by sampling cells, which has limited spatial scope and often sparse temporal resolution. The limited information that can be gathered from cell sampling also means that precise control of the cell culture process towards a desired outcome is also limited. This results in very poor efficiency, consistency, and functionality in many cell culture processes, particularly given variations from patient to patient, cell line to cell line, and process to process (including initial conditions). In summary, the spatio-temporal trajectory of cell culture processes is complex and depends on many initial conditions and in-process changes (e.g., cell migration, cell division, cell death, cell transformation), and are also hard to measure with high resolution. Thus there is a need in the art for systems and methods to accurately measure cell trajectories during a cell culture process and then dynamically alter the cell culture process based on the observed trajectories.

The systems and methods disclosed herein include a system configured for accurate measurements of spatial configurations of cells and/or cell characteristics and control of cells during a cell culture process. The implementations disclosed herein further include systems for large-scale, label-free global imaging of cell culture processes, with computational tools to map cell cultures and track them over time. Additionally, the implementations disclosed herein include methods for linking in-process label-free image-based data with genomic, transcriptomic, proteomic, epigenetic, phenotypic, and functional data (i.e., -omic data) on cells (either single cell or bulk), both in the spatial and non-spatial domain. The information on cells, cell colonies, and cell regions collected over time may be used to back-trace the cellular origin of cells that end up in target or non-target cell states. This allows for the identification of likely conditions that give rise to desired cell states, and optimization of cell culture processes by the system in future runs of the cell culture process by changing the conditions of the initial and intermediate cell culture process towards the identified conditions. In some implementations, once the cell culture process behavior has been measured and traced with the methods described herein, the observed/back-traced spatial and -omic trajectory behaviors may be used to inform a simulation of the cell population and cell culture process. Such a simulation may be used to optimize cell culture management processes, including but not limited to reinforcement learning models for the control of the cell culture process.

The cell culture systems disclosed herein may be configured to manage and spatially control cell culture processes by accurate, selective removal of cells at a large scale, and also configured to selectively deliver compounds to cells in a spatially-selective manner. The cell culture system may also be configured to acquire high resolution information on cell culture process trajectories and use the information to manage global cell culture process conditions or parameters (e.g., timing of reagents or harvest, etc.). The implementations disclosed herein may enable the collection of high resolution spatial, and by proxy transcriptomic or other -omic, trajectories in cell culture processes, which allow an increase in consistency, yield, purity, and functionality in cell culture output products. The implementations disclosed herein may be applied to both 2D and 3D cell cultures.

The implementations contemplated herein include the concept of a multi-label brightfield-to-spatial -omics prediction model for identifying and guiding cell state transitions. Most cell culture processes involve cell state transitions. For example, in reprogramming somatic adult cells transform into iPSCs, in differentiation iPSCs are transformed into functional target post-mitotic cell types, and in rejuvenation aged cells are transformed into epigenetically young cells. The stochastic nature of these cell state transitions can cause them to be error-prone, leading to incorrect states (e.g., partial or incomplete reprogramming, differentiation into an unwanted cell type, or transition into a cancerous state during transient reprogramming or rejuvenation). These transitions may be mapped to cell trajectories in a high-dimensional gene expression space (e.g., RNA or protein expression of cells). The cell trajectory along this space may be represented with a lower-dimensional set of marker genes that capture the trajectory accurately. Generally, non-spatial or -omics methods may be used on timepoint samples, followed by data analysis (including waddingtonOT, diffusion mapping, or other similar methods) to identify marker genes.

The spatial expression profiles of the sparse sets of markers may then be identified using a variety of methods, including multiplexed immunofluorescence that can measure spatial protein expression in fixed samples, multiplexed DNA/RNA-FISH methods that can spatially map DNA sequence changes or RNA expression changes, and gene editing cells to contain fluorescent tags or reporters linked to the expression of markers that allows spatiotemporal data collection on these markers from live cells. Collection of data linking the position of a cell or a cell neighborhood's position along a cell state trajectory to its spatial location may allow the training of predictive models inferring cell state based on label free images of cells. The model may be, for example, image-to image models with light microscopy inputs and multi-label smoothened spatial expression map outputs.

The predictive model may be used by a cell culture system to make process decisions based on the collected observations. For example, the cell culture chamber level cell statistics may be used to make chamber-level decisions around cell culture condition changes, termination of process, harvesting etc. The cell culture system may be able to identify cells that enter an unwanted state or are lagging in a starting state and remove them via cell removal tools (CRTs). Cell culture process intervention decisions (e.g., modulation of global conditions, spatial interventions) may either be made by human evaluation, a rule-based algorithm based on predictions, or an additional learned model (trained on human decision data or using a policy based on the predicted labels).

The implementations contemplated herein also include the concept of dynamically modulating the spatial distribution of cells to optimize the efficiency of cell culture processes. The spatial configuration of cells at the initiation or progression of a directed cellular state transition may have an impact on the efficiency of the targeted transition, whether this be differentiation, reprogramming, gene editing, or rejuvenation. Stochasticity in the initial spatial distribution of cells on a 2D surface, along with complex growth and motility dynamics can make it difficult for a human to observe the data and identify patterns implicating spatial features of cells to their transition efficiency. However, this stochasticity can also be a means to sample diverse spatial features and contexts for cells and cell regions. Combined with a defined spatial readout of transition efficiency, this can enable the generation of paired datasets that may be used by a machine learning model to hypothesize key spatial features of cell regions that encourage desired cell state transitions. Discovered relationships between initial spatial context of a cell and the resultant cell state after completion of a cell culture process may be used to design optimal initial spatial patterns of cells for maximum transition efficiency. The spatial pattern execution may be achieved, for example, by selective removal of cells by a CRT after a confluent or random initial seeding or by selective removal of extracellular matrix (ECM) by a CRT (after which cells are only able to attach to specific locations on the surface).

Due to the dynamic nature of cells (e.g., their motility and proliferation), it may be difficult to trace cells at a terminal point of a cell culture process back to earlier timepoints, especially if there are only a few timepoints at which the cells are imaged. This can be further complicated by the fact that the growth and motility of cells are dependent on their cell state. To solve this, inverse mathematical models conditioned on real cell density change data may be constructed to help predict or back-trace the spatial distribution of cells over multi-day cell culture processes. These models may be based on physical models of cell growth with parameter fitting, modified unbalanced versions of optimal transport models, or image or map based deep learning models.

In various implementations contemplated herein, a machine learning model may be developed to understand the spatial dispersal dynamics of cells over time during a cell culture process. For example, a model may take as input a time series of brightfield images and an annotated patch of cells in a first timepoint and the model may return a spatial probability distribution of cells derived from this patch at some intermediate or terminal timepoint. Conversely, such a model may be used to back-trace a patch of cells at an intermediate or terminal timepoint to a cell region in an earlier timepoint. Training this model for any general cell culture process may rely on sparsely mixing fluorescently labeled cells among otherwise unlabeled cells. The sparse labeled cells may be imaged via fluorescence and processed at each timepoint to generate ground truth data on the dispersal dynamics of cells during the given cell culture process. Such a cell flow prediction model may be applied as part of, for example, laser-based iPSC clonalization monitoring. Specifically, during the clonalization process a cell colony may be back-traced to a likely cell region at an earlier timepoint (e.g., the initial timepoint). Once the predicted source cell region falls below a defined threshold (e.g., less than or equal to a single cell area), the cell colony may be assumed clonal.

Various deep learning and other machine learning/artificial intelligence models may be used in conjunction with the various implementations disclosed herein. For example, in the use case of dynamic modulation of spatial distribution of cells described herein, a cell density and phenotype flow predictor may be trained in a self-supervised manner using time ordering as ground-truth from two-N frame brightfield (or other modality or other gene expression level maps) images collected from many experiments of the same cell culture process (e.g., multi-day image sequences of many wells/containers collected with the same cell culture process conditions). Depending on its architecture, such a predictor may then predict the next frame given one image as input (i.e., “sample” a next frame conditioned on a “first” frame) or predict the next N-k frames given the previous k label-free images as input (“sample” N-k next frames in the sequence given the previous k frames). The image encoder of the predictive model may start from a pre-trained visual transformer model or a pre-trained ResNet model. Through the course of flow model training, a pre-trained visual encoder may be fine-tuned to capture the visual patterns of spatial density diffusion or phenotype change dynamics of local cellular regions by virtue of observing many spatially and temporally correlated local regions in the cell culture process image sequences.

The learned flow predictor model may further be leveraged to compute a dense correspondence map (a.k.a. optical flow map) between two consecutive frames. Specifically, a reference and a target image may be passed through the model to compute feature vectors from a set of locations (e.g., pixel regions) on each frame. Then an affinity matrix may be computed which measures the embedding feature vector distances of potential correspondences between a given location on the reference frame to a set of potential matching locations on the target frame. Then the location with the nearest feature vector may be assigned to correspond to the location in the reference frame. Such a map would trace the correspondence of similar looking cell regions across time-points as long as the time-spacing is close enough and the cell phenotype and appearance characteristics do not change. Note that differentiating cells may not be tracked reliably with this technique. To predict flow vectors into differentiating regions in the target frame, a sparse correspondence map may be initially computed by requiring “strong” feature similarities between reference and target frame locations, and these strongly matching locations would constitute “anchor” locations between the two frames initializing a sparse flow vector field. A dense vector field may then be interpolated from this sparse field by assigning a flow vector to each location in the reference frame using vectors of the neighboring locations.

In some implementations, a dense correspondence map between two consecutive frames may be computed through an analytical flow vector calculation taking the growth rate of the clone into account (e.g., assuming radial growth into healthy matrix regions with a certain velocity). A hybrid approach may use such an analytical model to initialize a correspondence search area for the reference locations in the target frame, followed by the feature vector similarity-based assignment process described herein to resolve the final assignment. A fuzzy frame to frame correspondence map may also be calculated by avoiding hard assignments but fuzzy assignments to a group of target locations in the target frame.

In the use case of a multi-brightfield-to-spatial -omics prediction model for identifying and guiding state transitions, novel deep learning techniques may be used to align a latent vector space of images to a latent vector space of gene expressions. Deep learning techniques may be configured to discover semantic relationships that exist in different domains in self-supervised ways. For these techniques to be applicable, a dataset in which visual domain data which correlates with genomic data (or other expression/-omics data types) should be collected, albeit not in strict pixel to genomic-token correspondence. For example, a cell culture process experiment may be designed in which images of a certain spatial size are collected such that corresponding genomic expression data from the cell population observed in that image is available (e.g., a 96 well or smaller format size images and corresponding RNAseq data from that well from a particular time-point during a cell culture process). The latent vectors of image patches in that image may be mapped to latent vectors of the same dimension from gene expression data. A one-to-one correspondence of expression data from a single cell and a single cell observed in that patch is not required, given that an abundance of image-patch-to-population-expression-data may be collected.

In another implementation of the use case of a multi-brightfield-to-spatial -omics prediction model for identifying and guiding state transitions, a supervised deep learning framework may be used. For a supervised deep learning framework to be applicable, labeled images of cells that express certain identified gene profiles may be generated as matching ground-truth label images to the label-free images of a target cell culture. For example, certain gene expression profiles along a differentiation trajectory may be identified through optimal transport (OT)-type techniques or through expert designations. Then a cellular marking or gene-editing technique may be used to image a cell culture in corresponding relevant fluorescent channels which measure the expression levels of the tagged or marked genes. Given a sufficient number of paired brightfield and fluorescent images, a pixel-wise segmentation deep learning model may be trained to input label-free images and output the labeled channels as its predicted expression maps.

If a trajectory in the genomic expression domain can be learned through OT-type experiments, and a correspondence of image tokens to genomic profile tokens is established through the methods described herein, then a predictive model may monitor cellular regions in a cell culture container and the cell culture system may be configured to intervene at trajectories predicted to be undesired, through actions generated to impose a new spatial pattern in the cell culture container (e.g., through generation of a cell removal map). Examples of performing action generator model learning techniques that may be applied to these scenarios are described herein.

An example implementation of the systems and methods contemplated herein includes a cell culture system configured to perform a cell culture process (e.g., reprogramming, expansion, differentiation) with imaging at a plurality of timepoints. The cell culture system may be further configured to map regions of success (e.g., where cells of a target cell type are present in the final cell culture), trace back the origins of target and non-target cell types through a cell proliferation and motion model, and find spatial configurations at an earlier timepoint that likely give rise to cells of the target cell type. In future cell culture process runs, the cell culture system may be configured to spatially shape the cell culture using a combination of proliferation, selective cell removal, and/or selective removal of ECM to steer/shape cell growth to match the spatial configurations found to be successful.

Another example implementation of the systems and methods contemplated herein includes a cell culture system configured to perform a cell culture process (e.g., reprogramming, expansion, differentiation) and capture with label-free images at a plurality of timepoints during the cell culture process. The cell culture system may be further configured to map cell phenotype regions at a final timepoint, using labeling and then trace back the spatial origins of phenotype regions at one or more prior timepoints. The cell culture system may be further configured to utilize the image data from the traced regions (e.g., raw image data, or single timepoint maps generated from image data), in a time series, as an input to a model that predicts phenotype from this time series of label-free images. The cell culture system may be further configured to use the predictive model to make label-free predictions on phenotype by region in the final cell culture, and optionally selectively remove cells based on this phenotype map. The cell culture system may also be configured to modulate or perturb cell culture conditions as described herein in conjunction with image acquisition to collect additional information about the cell culture.

Another example implementation of the systems and methods contemplated herein includes a cell culture system configured to perform a cell seeding and expansion process across multiple cell lines. The cell culture system may be further configured to trace cell proliferation and mobility via optimal transport methods, use this tracing information to build a parametrized model to predict spatial cell proliferation, and predict the best confluence and spatial patterns in expanded cells from the model. The cell culture system may be further configured to adjust cell seeding conditions based on predicted behavior and to calculate model parameters that best describe proliferation of the present cell line after seeding and initial proliferation observation. Using the developed model, the cell culture system may be configured to determine an optimal combination of proliferation and cell removals by CRT, and optionally selective ECM removal, to achieve an optimal confluence state at end of process. This loop may be applied to multiple differentiation protocols in which differentiation or trans-differentiation processes start from cells at various degrees of confluence and/or colony arrangements.

Another example implementation of the systems and methods contemplated herein includes a cell culture system configured to perform a cell culture process (e.g., reprogramming, expansion, differentiation) and remove regions of cells at various timepoints, and in various spatial conditions, using a CRT. The cell culture system may be further configured to track the re-growth of cells into/around the removed cell regions, with the use of an optimal transport or other motion and proliferation model and use this tracked data to model cell proliferation into removed regions at different timepoints, local conditions, and local phenotypes. The cell culture system may be further configured to use this model to establish the timing and size at which cell regions are to be removed during cell culture process, to remove undesirable cells, and to allow optimal re-growth of desirable cells into the removed regions. During such processing, the cell culture system may be further configured to track the re-growth of cells as a way of characterizing the local cell state, phenotype, and functionality (a form of condition modulation for establishment of cell characteristics, as described herein). For example, this approach may be applied to the differentiation and maturation of retinal pigment epithelium (RPE) cells to establish timing and spatial scope for using CRT to remove cells predicted to not yield optimally functional RPEs and let adjacent RPE populations grow into these removed areas.

Another example implementation of the systems and methods contemplated herein includes a cell culture system configured to perform a cell culture process in which 2D patches of cells are detached from the cell culture surface to form 3D structures (for example, for the purpose of differentiating cells or organoids). The detachment may be made by over-density in a confined space, change of media, addition of a matrix material on top of the cells, and/or use of a laser to cause detachment of the cells from the cell growth surface. The cell culture system may be further configured to trace successful and unsuccessful 3D structure outcomes back to 2D spatial history (including area and shape histories) and develop a model for optimally and dynamically controlling the area and shape of 2D colonies based on growth trajectories, areas, and shapes. Controlling the cell culture may be achieved by, for example, adjusting growth times in 2D (e.g., for an entire cell culture or on a colony-by-colony basis, assuming the detachment of cell sheets to form 3D structure may be controlled via a spatially-selective tool), removing cells using a CRT in order to slow the area growth or to shape the growth into an optimal formation, or removing ECM to shape growth, slow growth, and/or cause the detachment of the 2D structure to form the 3D structure.

Another example implementation of the systems and methods contemplated herein includes a cell culture system configured to perform cell colony management on a single surface of a cell culture container using a CRT to continuously remove cells such that the colony remains at a healthy density and size as described herein. The cell culture system may be further configured to measure the outcome of such in situ colony management, in which the outcome may include but is not limited to vector load of a vector to be cleared by continuous colony management, clonality, level of pluripotency, level of differentiation, phenotype, transcriptome, proteome, etc. The cell culture system may be further configured to trace back the spatial trajectory of the cells in the final colony configuration to determine their position within the colony over the course of colony management and to measure factors such as local image patch data, local cell density, distance to colony edge, local cell division rate, and local predicted characteristics via deep learning mapping models. The cell culture system may be further configured to establish relationships between the spatial condition trajectory of the cells and their outcome, and to revise colony management protocols according to optimal measured spatial trajectories. The relationship establishment and cell colony control optimization may be entirely guided by a reinforcement learning model coupled with at least one of outcome assay data and simulated run data. This process may be used for iPSC reprogramming, for example, to optimize properties such as vector clearance, pluripotency, reduction in differentiated cells, clonality, and functional, epigenetic and transcriptomic properties of the resulting colonies.

The systems and methods contemplated herein for measuring and managing cell trajectories may enable various cell culture process operations and optimizations. For example, the implementations disclosed herein may be used to, among other things, (1) optimize cell or cell cluster seeding densities in cell culture containers with knowledge that proliferation and selective cell removal may be used to achieve an optimal spatial configuration; (2) optimize spatial ECM removal or deposition to achieve optimal patterning; (3) selectively remove cells or pattern ECM during cell proliferation to guide spatial distribution of cells, including cell structure size, shape, and density; (4) selectively remove cells to steer phenotypic composition of a cell culture on a global and/or local level as a function of timepoint in a cell culture process; (5) selectively remove cells or pattern ECM to prepare specific regions of cells for harvesting for the purpose of downstream processing or analysis; and (6) selectively remove cells to purify a cell population to one or more target cell types.

The systems and methods contemplated herein may be applied to a range of cell culture processes including but not limited to: expansion, purification, reprogramming, differentiation, gene editing, trans-differentiation, drug screening, rejuvenation including but not limited to partial reprogramming approaches, cell sheet patterning, and 3D cell structure formation, including combinations of these.

Cell trajectories in spatial or -omic space described herein may include those that result in cells dying. This may be a negative if cells were meant to transform in a target cell type. However, it may also be a positive outcome if the cells are contaminating cells or non-target cells. For example, a change in global conditions could cause a larger number of non-target cells to die off, which can leave more space for proliferation of target cells. In another example, a spatial treatment with a CRT removing cells or removing ECM could cause selective death among non-target cells or select for certain cell trajectories. However, some CRT operations may make colonies that are too small in one dimension may result in a die-off of cells that were destined to become target cells. The back-tracing, learning, and reinforcement learning models described herein may be used to identify these relationships faster than by multi-factorial experimentation.

Spatial interventions of cell cultures as disclosed herein may be performed by various methods including but not limited to removal of selected cells (including adherent, partially adherent, and non-adherent cells) and removal of extracellular matrix from the surface of the growth container in a manner that affects cell movement or proliferation (in some cases differentially based on cell type or characteristics).

Spatial interventions may be performed with various tools, including cell removal tools (CRTs) as described herein. These cell removal tools include but are not limited to laser-based tools (including direct laser removal of cells, laser removal by use of a temperature-sensitive layer, laser removal by use of a dye in the cell media, laser removal by direct absorption in the media, laser removal by nanoparticle absorption, laser removal by laser-absorbing film absorption, or laser removal by bubble formation), mechanical tools (e.g., magnetically-coupled mechanical tools), focused ultrasound, and spatially controllable electric fields.

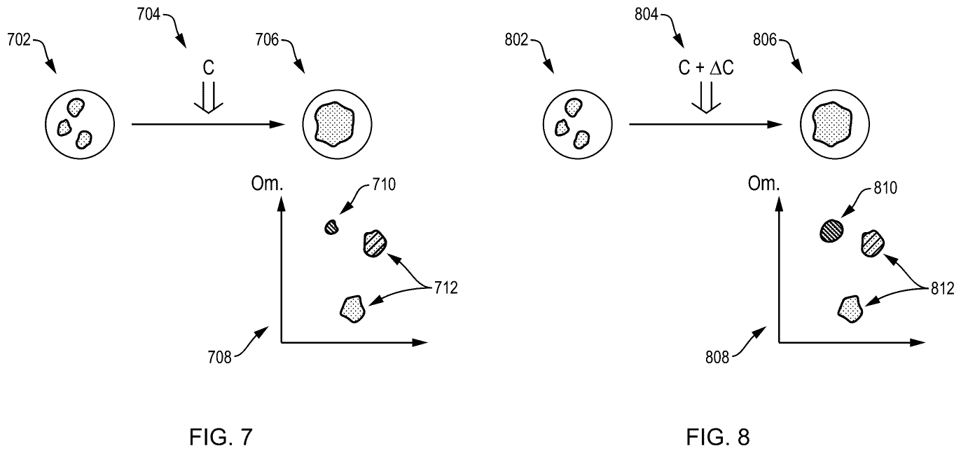

FIG. 7 is a diagram illustrating a prior approach to a cell culture process. An initial cell population 702 (e.g., a cell culture in a cell culture container) is exposed to global conditions “C” 704. The global conditions 704 may include, for example, a series of reagent changes, media operations, passaging operations, temperature changes, etc. that the cell population 702 is exposed to globally in the cell culture container. The global conditions 704 may be changed at a set of timepoints, resulting in an output cell population 706.

Graph 708 is a 2D depiction of -omics measurements of the output cell population 706. The output cell population 706 is shown in a highly simplified form as a cluster that represents a target phenotype 710 and two clusters that are non-target phenotypes 712. In general, the -omic map of a cell population is typically very high-dimensional, although it may be projected to 2- or 3-dimensional maps such as the one shown in FIG. 7 for ease of understanding. The goal of a cell culture process (such as reprogramming, expansion, or differentiation) may be to produce as many of the cells with the correct phenotype 710 as possible, and to minimize impurities in the form of non-target phenotypes 712.

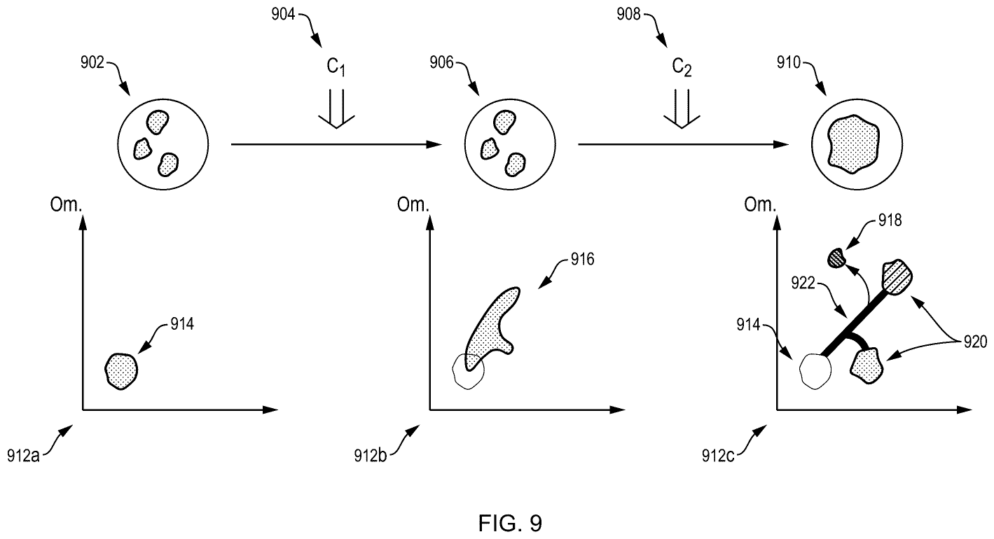

FIG. 8 is a diagram illustrating another prior art approach to managing a cell culture process. An initial cell population 802 (e.g., a cell culture in a cell culture container) is exposed to modified global conditions “C+ΔC” 804, modified with respect to the global conditions 704 in FIG. 107. Such modifications may include changing the timing of operations, the concentrations of various fluids, reagents, and other compounds, changing cell growth conditions, etc. Application of the modified global conditions 804 results in an output cell population 806 that is different from the output cell population 706 of FIG. 7 due to the difference in global conditions.

Graph 808 is a 2D depiction of “-omics” measurements of the output cell population 806. As compared to cell culture process depicted in FIG. 7 with original global conditions 704, the modified global conditions 804 may have resulted in an increase of cells having a target phenotype 810 while reducing the population of cells having non-target phenotypes 812. In prior art practice, global conditions may be modified many times to try and optimize a cell culture process. Sometimes, this experimentation is done with a matrix of conditions to be tried, or with a designed experiment, or with search algorithms including artificial intelligence/machine learning based approaches. This prior art method is used for many current cell culture processes. Sometimes cell culture processes are broken into multiple phases, each of which is independently optimized.

However, this approach may ignore the importance of trajectory within the phases that are not captured in the measured cell culture state. This simple, often-used optimization method also does not incorporate time-series elements, nor does it relate results to spatial relationships. In addition, multiple runs with multiple input cell lines are required to capture line-to-line variability. Furthermore, cell line specific behavior may not be evident until the endpoint measurement of the cell product, leading to low yields and low run-to-run and line-to-line consistency.

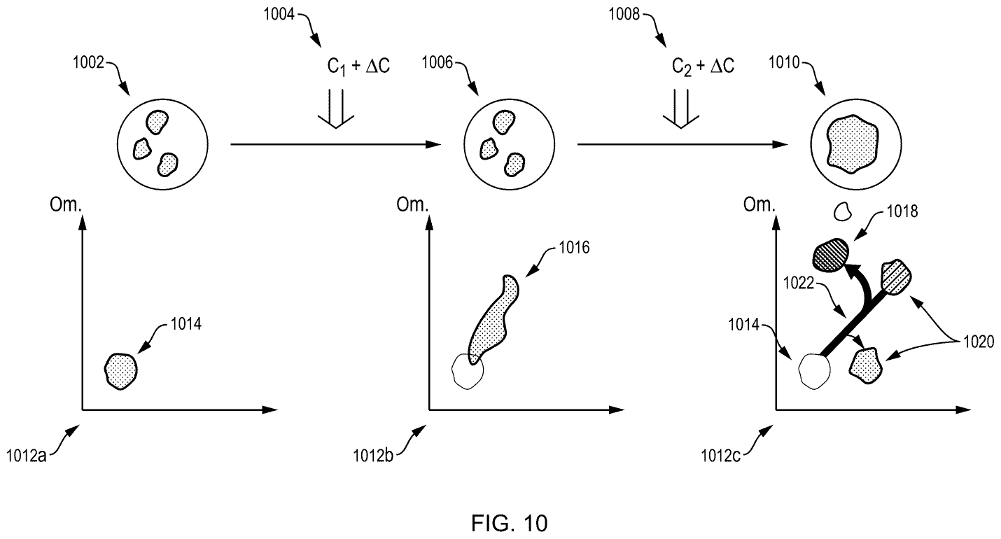

FIG. 9 is a diagram illustrating a multi-step prior art approach to managing a cell culture process. In this approach, the cell culture process may be characterized with -omics measurements at multiple stages to get an understanding of the -omic trajectory of the cells and thereby gain a better understanding of how to steer the process, rather than just optimizing based on the endpoint state. An initial cell population 902 (e.g., a cell culture in a cell culture container) may be processed with a first set of global conditions “C1” 904, resulting in an intermediate cell population 906. The intermediate cell population 906 is then processed using a second set of global conditions “C2” 908, resulting in a final cell population 910.

Maps 912a-c are -omic maps that correspond to the multi-step cell culture process shown in FIG. 9. Most -omics measurements cannot be made across multiple stages on the same cells because most measurement processes are destructive. Therefore, the -omic maps 912a-c may be representative of different cell populations at each stage rather than representing a consistent cell population throughout the whole process. In some cases -omics data may be measured across stages via live cell stains or engineered cell lines expressing fluorescent markers, but these provide very sparse data compared to other -omics techniques such as DNA sequencing, RNA sequencing, ATAC sequencing, methylation sequencing, proteomic measurements, or single-cell versions of such measurements.

A sample of the initial cell population 902 may be measured and mapped to phenotype space 914 within an initial -omics map 912a. A separate sample from the intermediate cell population 906 may be measured and mapped to phenotype space 916 within an intermediate -omics map 912b. A change in the -omic state can be seen between maps 912a and 912b, caused by the application of global conditions 904. A separate sample from the final cell population 910 may be measured and mapped to a final -omics map 912c. The final -omics map 912c includes an outline of the initial phenotype state 914, a population of cells with target cell state 918, and populations of cells with non-target cell states 920.

From the initial, midpoint, and final -omic maps 912a-c, an -omic trajectory 922 may be reconstructed that is a temporal map showing branches, merges, and residual populations of the -omics state of the cell population as it progresses through the cell culture process. The purpose of the -omics maps 912a-c is to gain insight into how and when to modify global conditions to guide a larger proportion of the initial cell population 902 to the target cell state 918. In practice, the trajectory information may be gathered by multiple runs of the cell culture process up to one or more timepoints where -omic information is measured, often across diverse cell lines to capture line-to-line variability as well as run-to-run variabilities. For the purpose of tracing -omic trajectories from disparate runs and from sparse timepoints, an optimal transport (OT) solution may be applied that predicts likely cell trajectories and may be used to interpolate between timepoints. The optimal transport technique as applied to single cell RNAseq data is described in Schiebinger, Geoffrey et al., “Optimal-Transport Analysis of Single-Cell Gene Expression Identifies Developmental Trajectories in Reprogramming,” Cell 176, 928-943 (2019), which is hereby incorporated by reference in its entirety.

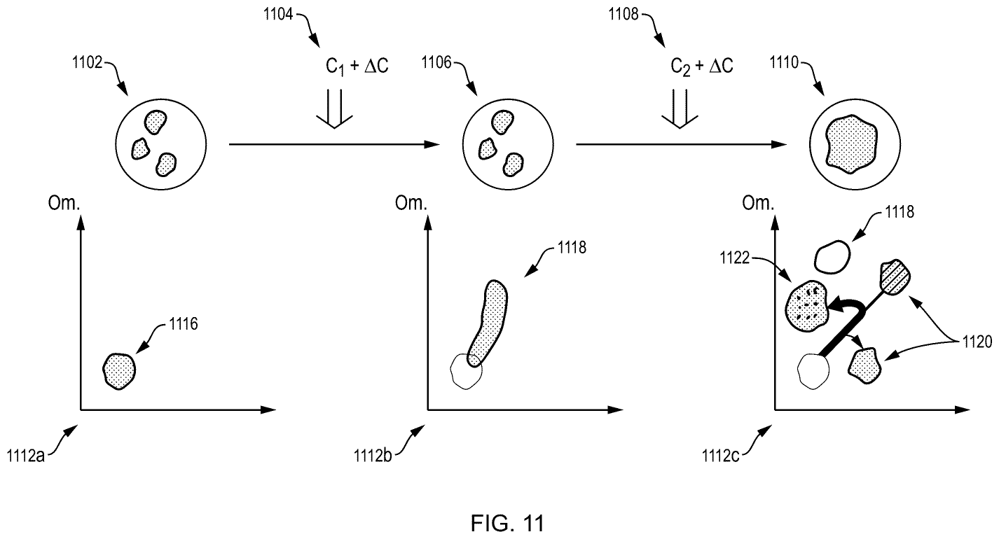

FIG. 10 is a diagram of another multi-step prior art approach to managing a cell culture process. FIG. 10 may depict a modified cell culture process that is constructed using information gained from the -omic trajectory mapping of the cell culture process depicted in FIG. 9. An initial cell population 1002 (e.g., a cell culture in a cell culture container) may be processed with a first set of modified global conditions “C1+ΔC” 1004, resulting in an intermediate cell population 1006. The intermediate cell population 1006 is then processed using a second set of modified global conditions “C2+ΔC” 1008, resulting in a final cell population 1010.

Information gained from the -omic maps 912a-c may allow more precise targeting, in time and conditions such as reagents, factors, etc. to control cell fate trajectory in the modified process shown in FIG. 10. Maps 1012a-c are -omic maps that correspond to the modified multi-step cell culture process shown in FIG. 10. A sample of the initial cell population 1002 may be measured and mapped to phenotype space 1014 within an initial -omics map 1012a. A separate sample from the intermediate cell population 1006 may be measured and mapped to phenotype space 1016 within an intermediate -omics map 1012b. A separate sample from the final cell population 1010 may be measured and mapped to a final -omics map 1012c. The final -omics map 1012c includes an outline of the initial phenotype state 1014, a population of cells with target cell state 1018, and populations of cells with non-target cell states 1020. The process depicted in FIG. 10 results in more cells ending up within the target cell state 1018 as compared to the original process depicted in FIG. 9, and also fewer cells ending up within the non-target cell states 1020. Thus, by modifying the global conditions an -omic trajectory 1022 of the cells may be steered to a more desirable outcome. This technique has been used to successfully predict which globally added compounds can steer the cell culture process to achieve a higher yield of target cell types.

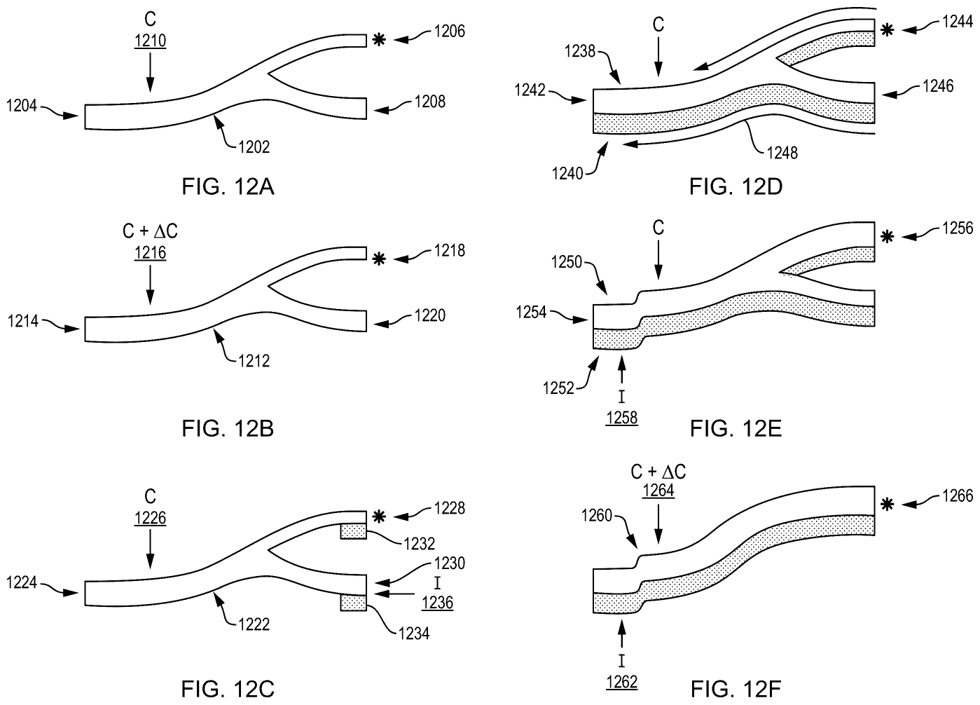

FIG. 11 is a diagram of another multi-step prior art approach to managing a cell culture process. In this example, a cell culture process may be performed with a different set of global condition modifications than illustrated in FIG. 10 that are designed to “steer” cells along a trajectory heavily favoring a target cell type, but also illustrates the potentially complex relationships that can make naive strategies fail. An initial cell population 1102 (e.g., a cell culture in a cell culture container) may be processed with a first set of modified global conditions “C1+ΔC” 1104, resulting in an intermediate cell population 1106. The intermediate cell population 1106 is then processed using a second set of modified global conditions “C2+ΔC” 1108, resulting in a final cell population 1110.

Maps 1112a-c are -omic maps that correspond to the modified multi-step cell culture process shown in FIG. 11. A sample of the initial cell population 1102 may be measured and mapped to phenotype space 1114 within an initial -omics map 1112a. A separate sample from the intermediate cell population 1106 may be measured and mapped to phenotype space 1116 within an intermediate -omics map 1112b. A separate sample from the final cell population 1110 may be measured and mapped to a final -omics map 1112c.

The final -omics map 1112c shows that with the modified global conditions 1104 and 1108, no cells end up within the target cell state 1118. Rather, the cells are steered away from known non-target cell states 1120 that typically arise in the cell culture process but toward another different non-target cell state 1122. Such a result can occur if, for example, a cell population is forced into a trajectory that is not feasible (or results in cell death or loss of cell health), or there are interdependencies between cell types, meaning a non-target cell population is required for at least a portion of the cell culture process to support the cells that ultimately result in the target cell type.

Thus there is a clear need in the art for more sophisticated control over cell trajectories, and commensurate models for optimizing cell culture processes by controlling such trajectories. Various machine learning and artificial intelligence models have been applied to the prior art purely -omic models to make better predictions and optimizations. However, the shortcomings of these approaches are that they are dependent on snapshots of disparate cell cultures, often with large time gaps and very different starting conditions and sometimes cell culture conditions.

Moreover, since cell culture processes must often be run label-free, or with very limited markers, they must run “open loop” from end to end (possibly with some sampling at intermediate points). Dynamic control of the cell culture process to account for run-to-run or line-to-line variability was not possible. Finally, conditions applied to cell culture processes are applied globally in almost every case, removing the possibility of dynamic control based on spatially localized cell characteristics.

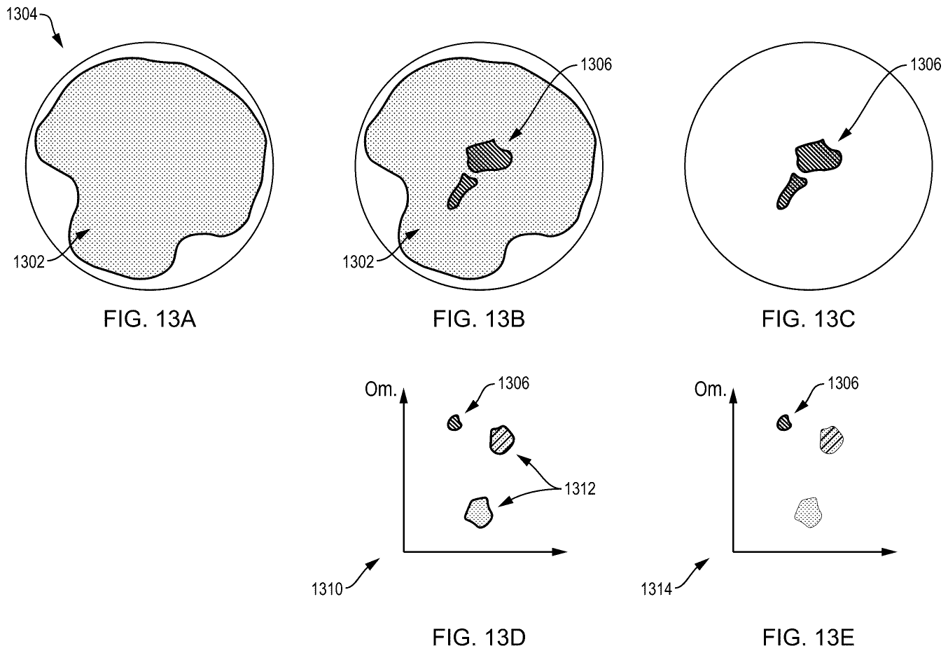

The systems and methods disclosed herein overcome the shortcomings of the prior art by providing a way to continuously monitor a cell population through a cell culture process to capture its complete -omic trajectory and then use the information to determine which cell conditions give rise to desired output cell states. This knowledge may be used to optimize future cell culture processes and improve desired cell yield and efficiency. FIG. 12A-F are diagrams illustrating the alteration of -omic trajectories of cells in accordance with various implementations. FIGS. 12A-F are high-level conceptual illustrations of how -omic trajectories may be measured and used to improve cell culture processes. Specifically, -omic trajectories are influenced by initial conditions (e.g., cell line, cell spacing, etc.) and global conditions applied to the cells (e.g., media, transcription factors, chemical signals, etc.) to push some of them to target cell types. These global conditions may be changed to alter -omic trajectory. The -omic status of cells in a cell culture may be measured and the information used to purify cells at endpoints or intermediate stages of a cell culture process (e.g., by removing cells using a cell removal tool). Spatial trajectories of cells (rather than static snapshots) may be used to trace back -omic trajectories of cells, and make better cell fate predictions in the forward direction from spatial configurations or changes over time. In addition, knowing the relationships between spatial configurations and -omic trajectories, it is possible to make proactive changes to spatial configurations using selective cell removal tools in a proliferative cell culture. Finally, combinations of proactive spatial shaping, global condition modulation, and remedial removal of non-target cells by spatial shaping may lead to highly efficient production of high-purity, highly-functional cells.

FIG. 12A shows a graphical representation of an -omic trajectory 1202 going from a starting cell population 1204 to a target cell population 1206 and a non-target cell population 1208, the -omic trajectory 1202 guided by global conditions “C” 1210.

FIG. 12B shows a graphical representation of an -omic trajectory 1212 of an initial cell population 1214 guided by modified global conditions “C+ΔC” 1216 (as opposed to the original global conditions 1210 in FIG. 12A). The modified global conditions 1214 results in a larger target cell population 1218 and a smaller non-target cell population 1220 compared to the original cell culture process shown in FIG. 12A.

FIG. 12C shows a graphical representation of an -omic trajectory 1222 of an initial cell population 1224 guided by global conditions “C” 1226 to a target cell population 1228 and a non-target cell population 1230. The -omic status of the target cell population 1228 and the non-target cell population 1230 may be ascertained, classified, or predicted via spatial information 1232 and 1234 respectively. The spatial state of the cells is driven by the -omic state, and therefore the mix of cells and spatial location of cells may be predicted from imaging, including label-free imaging. In such a system, a spatially-selective intervention “I” 1236 may be applied to physically remove non-target cells.

FIG. 12D shows a graphical representation of a combined -omic trajectory 1238 and spatial trajectory 1240 for an initial cell population 1242 that leads to a target cell population 1244 and a non-target cell population 1246 after global conditions “C” are applied. Once both trajectories 1238, 1240 are established, a back-tracing process 1248 may be used to uncover interrelationships between the two trajectories 1238, 1240. The interrelationships may be used to build a predictive model to improve future cell culture process runs.

FIG. 12E shows a graphical representation of use of a predictive model to steer an -omic trajectory 1250 (and spatial trajectory 1252) of an initial cell population 1254 in favor of a target cell population 1256 by applying a selective spatial intervention “I” 1258 that shifts the spatial trajectory 1252, and as a result shifts the -omic trajectory 1250 of the initial cell population 1254 as well.

FIG. 12F shows a graphical representation of an -omic trajectory 1260 that is optimized using both spatial interventions “I” 1262 and changes in global cell culture conditions “C+ΔC” 1264 to achieve an even higher efficiency in producing a target cell population 1266.

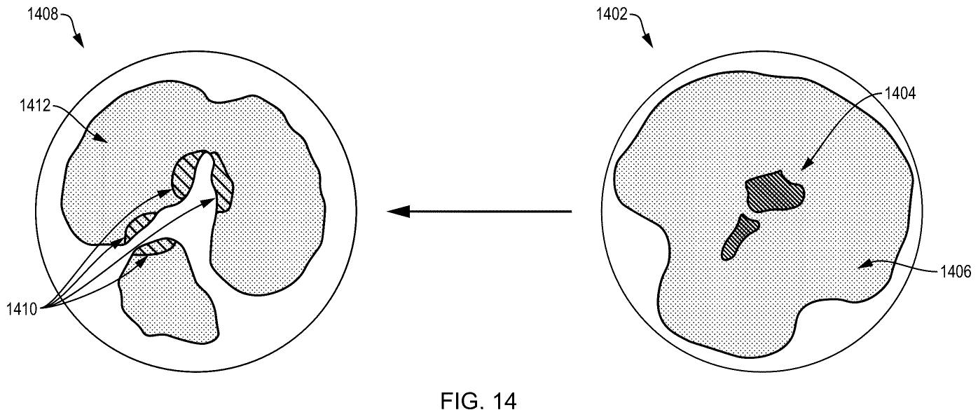

FIGS. 13A-E are diagrams illustrating the use of spatial and -omic relationships to manage a cell culture process in accordance with various implementations. FIG. 13A illustrates the endpoint of a cell culture process, with a cell culture 1302 inside of a cell culture container 1304. The cell culture container 1304 may be, for example, an open well in a microwell plate, a petri dish, a flask, or a fluidic cassette (including one that is amenable to multi-patient cell culture processing in a shared environment). Typically, only label-free imaging may be used to image the cells. As a result, a cell culture system may rely on artificial intelligence models to map cell phenotype using only brightfield images. Such a mapping may be produced from a single timepoint image (relying on morphological features) or may be produced from a time series of images that allows observations of regional cell dynamics (including but not limited to cell proliferation, cell migration, changes in cell morphology, cell death). In some implementations, a time series of images may be matched with modulated cell culture conditions, as described herein.

FIG. 13B illustrates a mapped version of the cell culture shown in FIG. 13A, with a map generated via a model from a label-free image as described herein. Regions having a target cell type 1306 are distinguished from the rest of the cell culture 1302. FIG. 13C illustrates the cell culture 1302 after an automated cell removal tool (CRT) is used to remove non-target cells from the cell culture 1302, leaving only regions with the target cell type 1306 and an otherwise empty area 1308.

FIGS. 13D-E illustrate the -omic maps corresponding to FIGS. 13B-C respectively. -Omic map 1310 in FIG. 13D shows the location of the target cell type 1306 within the -omic space, as well as the locations of non-target cell populations 1312. Spatial mapping may be performed in various ways known to persons of ordinary skill in the art, including generating a correspondence between image features and an -omic map as shown herein. After removal of cells using the CRT, -omic map 1314 in FIG. 13E shows that the cell culture has been purified and the target cell population 1306 remains without the non-target cell populations. While a purified cell population results, the number of cells may still be small, particularly if the mapping and CRT is performed with a bias towards removing any non-target cell types. For example, in a differentiation process, a bias may be applied to remove any residual pluripotent cells, at the expense of removing target differentiated cells. In some implementations, cell mapping and CRT operations may be performed in a manner that results in a pure, large population of target cell types, which represents an overall higher cell culture process efficiency.

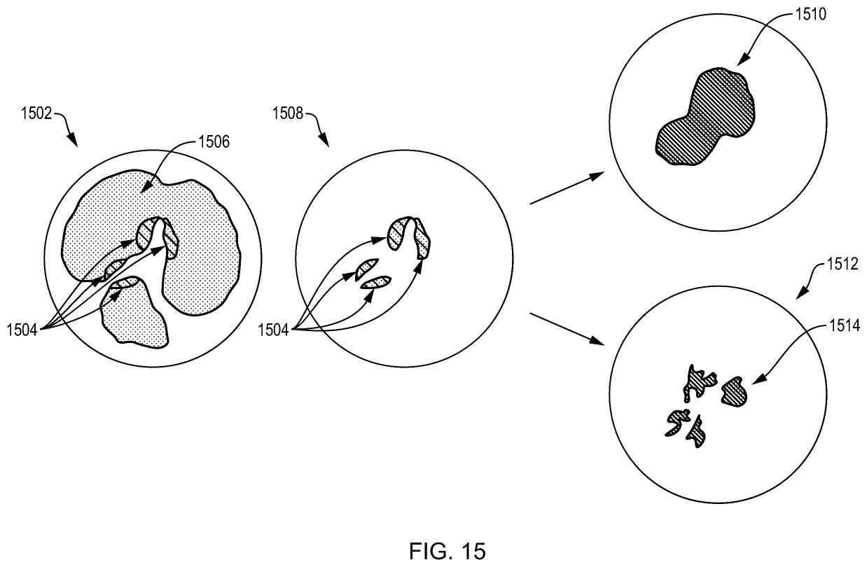

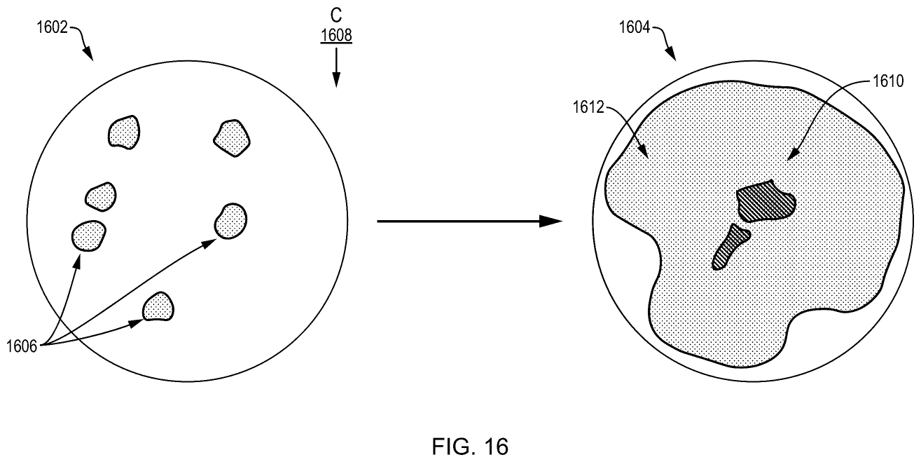

Various implementations disclosed herein include a multi-step approach for improving cell culture processes over multiple runs utilizing spatial/-omic mapping, illustrated by FIGS. 14-15. The first step of this approach is training a predictive model, which includes (1) capturing a time series of the cell culture process, with at least two timepoints; (2) spatially mapping the final cell populations at the final timepoint to create a map of target cell type regions and non-target cell type regions (this may be done with label-free images turned into labeled maps as described herein, or directly from labeled images such as immunofluorescently-labeled cell cultures); (3) using a spatial back-tracing algorithm within the time series to trace back the cells corresponding to the target cell regions in the final population to an earlier timepoint; and (4) creating a model that is able to map these “pre-target” regions from label-free images or image time series, in a manner described herein.

The second step in the multi-step approach is running further cell culture processes using the trained predictive models. This step includes (1) at a timepoint early in a cell culture process, creating predictive maps of pre-target cell regions within the cell culture; and (2) using a CRT to remove regions that are not identified as pre-target regions (i.e., not predicted to become target cells), thereby allowing more rapid proliferation of regions that are predicted to become target cell types. The next step in the multi-step approach is evaluating and purifying the cell culture process, which includes (1) creating a map of target and non-target cell regions at the final timepoint; (2) optionally, using this data and back-tracing as described herein to further train the predictive model for pre-target cells; and (3) using this map to guide the CRT to remove residual non-target cells from the final cell population. In this manner, predictive modeling may be used to produce a pure output cell population from an initial cell population, with an increased yield than would be possible with endpoint purification only. In some implementations, endpoint purification may be done with chemical or biochemical techniques rather than CRT. However, using CRT removal operations at an earlier timepoint in the cell culture process may increase output cell yield.

FIG. 14 is a diagram illustrating training a predictive model with spatial maps in accordance with various implementations. A cell culture process may be completed on a cell culture in a cell culture container, resulting in a final cell population 1402. The final cell population 1402 is mapped to spatially distinguish target cell populations 1404 and non-target cell populations 1406. For training purposes, this map may be created with labeled cell cultures (e.g., immunofluorescence, FISH, other labels, or combinations of in situ labeling for spatial context, and assays performed on cells after removal from the cell culture container, including but not limited to single cell sequencing techniques). Alternatively, a model trained to map target versus non-target cell regions from label-free image data may be used. The origin of target and non-target cells is then traced spatially to cell population 1408 at an earlier timepoint. The earlier cell population 1408 includes regions 1410 that ultimately became the target cell population 1402, and regions 1412 that became non-target cell populations 1406.

Back-tracing of cell populations may be done in various ways. In a labeled cell culture, which is typically only usable for training, it is possible to trace individual cells that express a fluorescent protein, such as a nuclear protein. Such tracing requires both adequate spatial resolution and time resolution but can provide very accurate correspondences, as well as additional information such as cell mobility and cell proliferation rate (versus local conditions) for various cell types. However, such tracing has significant overhead, and may be possible to run on only a few cell cultures for training purposes. For more disparate timepoints, a modified optimal transport (OT) solution may be used to trace likely regions of origin. This may be done with labeled cells, or with unlabeled cell cultures in which cell density may be mapped. OT solutions to find correspondence require fewer timepoints, and also may be used to generate intermediate timepoint maps. In the simplest case, an OT solution may be used with cell confluence maps only (no cell density information) and a map of the target cell type regions in the final cell culture to trace back likely origins to the pre-target cell regions in a prior timepoint.

Once regions have been mapped using back-tracing, the resulting map of pre-target cell type regions 1410 and non-target regions 1412 may now be used as ground truth for a model that predicts these regions from label-free image data. As described herein, these label-free images may be from a variety of modalities, and may include single timepoint or time series arrays that show local cell dynamics. In such a manner, predictions of regions that will result in target cell types may be mapped.

FIG. 15 is a diagram illustrating management of a cell culture process using a predictive model and selective cell removal in accordance with various implementations. The predictive model may be generated using the method described with reference to FIG. 14. FIG. 15 shows a cell culture 1502 at a first timepoint, early in the cell culture process. The cell culture 1502 is mapped using a predictive model that translates label-free images into a predictive map, predicting regions 1504 that will likely have a trajectory towards target cells at the endpoint of the cell culture process, and regions 1506 not likely to result in target cells at the endpoint of the cell culture process. A CRT is used to remove cells in the regions 1506 not predicted to become target cell types, resulting in cell culture 1508. The resultant cell culture 1508 leaves the pre-target cell regions 1504 isolated and able to expand freely in the cell culture container.

In some cases, the cells in the pre-target cell regions 1504 can rapidly proliferate into large population of target cell types 1510 in an otherwise empty cell culture container. The result is a pure, high yield population of the desired output cell population. However, there are some cases in which cells proliferate and mature into target cells only when surrounded by “supporting cells,” in which case this strategy may lead to a cell culture 1512 at the endpoint that has a large number of cells with non-target cell type 1514 (e.g., unhealthy cells or cells of the wrong type). In such cases, a more sophisticated approach may be required to control the -omic trajectory of cells via spatial means, as described herein.

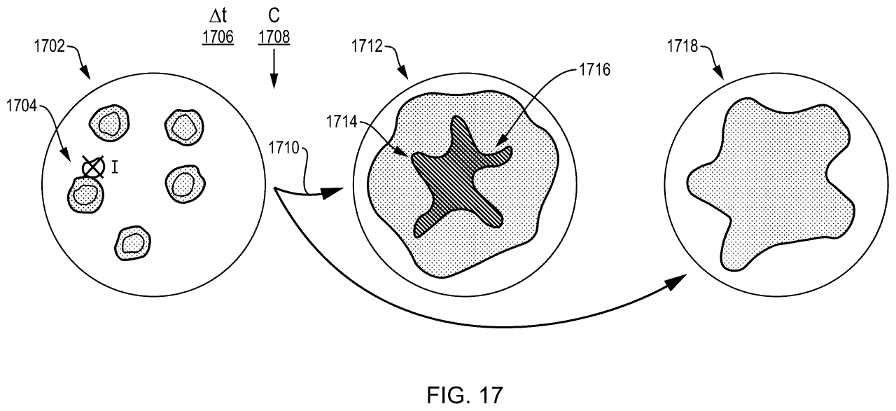

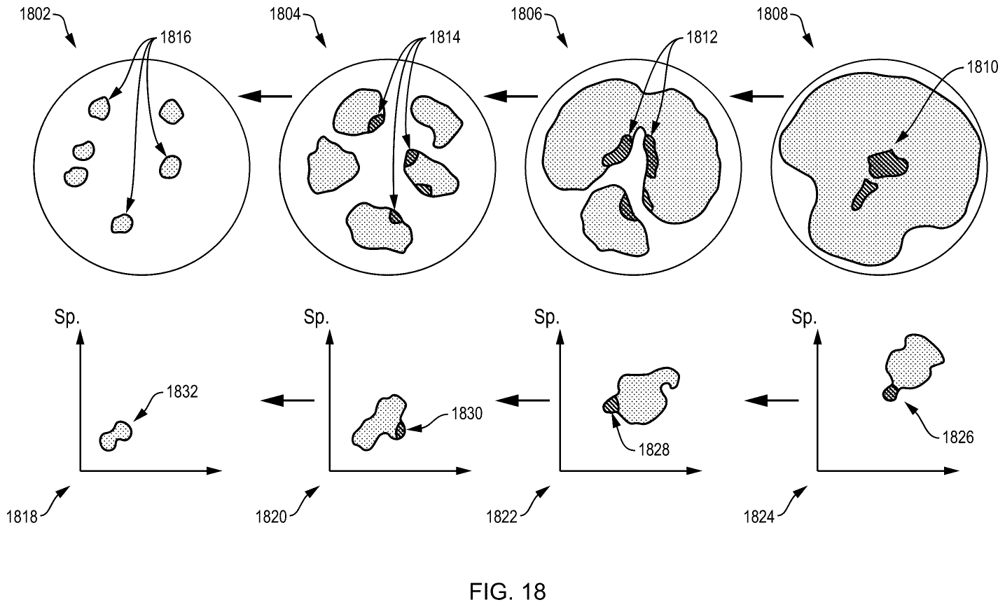

Further implementations disclosed herein include using spatial control of cell cultures to optimize the phenotypic outcomes of a cell culture process, as illustrated in FIGS. 16-17. Initial cell culture conditions, including spatial arrangements of cells in a cell culture container, often have a strong impact on the efficiency and consistency of various cell culture processes, including differentiation from pluripotent cells.

FIG. 16 is a diagram illustrating observation of the initial spatial configuration of a cell culture process in accordance with various implementations. FIG. 16 depicts a starting timepoint 1602 of a cell culture and an ending timepoint 1604 of the cell culture undergoing a cell culture process. At the starting timepoint 1602, the starting cells of the cell culture may be passaged in a manner to form cell colonies 1606 randomly distributed across the cell culture container. The cell colonies 1606 have a specific starting spatial configuration, which may include but is not limited to colony sizes, colony shapes, total number of colonies in the cell culture container, colony-to-colony distances, cell densities within colonies, neighboring cell types, etc. A set of global conditions “C” 1608 is applied to the cell culture to progress the cell culture process until the ending timepoint 1604.

At the ending timepoint 1604, the resulting cell population includes target cell types 1610 and non-target cell types 1612. The purity of the cell culture (e.g., number of target cells over total cell count) and the efficiency of the cell culture process (e.g., number of target cells over input cell count) may be calculated. The cell culture process may be run for many observations, with random initial conditions, or somewhat controlled initial conditions, including cell seeding density and when the cell culture process is commenced (for example, when factors that drive differentiation are introduced into the cell culture). Timing may be used as a gross control over spatial configuration when proliferative cells are used because cell colonies will grow or become more dense over time. The results of multiple cell culture process runs may be used to train a predictive model that associates cells that end up as the target cell type with their initial spatial configuration by back-tracing the cells through the cell culture process. Thus the predictive model may identify certain initial spatial configuration parameters that fosters cell growth towards the target cell type.

FIG. 17 is a diagram illustrating management of a cell culture process using a predictive model trained on initial spatial configurations in accordance with various implementations. The predictive model may be trained according to the example described with reference to FIG. 16, and the predictive model may be used to revise a cell culture process to optimize outcomes by controlling initial conditions. A cell culture system may alter the initial spatial configuration of a cell culture 1702 before the cell culture process is initiated. For example, the cell culture system may be configured to use a CRT to remove a cell colony 1704 from the cell culture 1702 to achieve more uniform cell colony spacing. In addition, a time delay Δt 1706 may be introduced before applying global conditions “C” 1708 to the cell culture 1702, which may encourage the cell colonies to grow to a larger size. The optimized initial conditions may cause the cell culture process to follow a different trajectory 1710 (as opposed to the trajectory illustrated in FIG. 16), resulting in a final cell culture state 1712 with a higher quantity of cells with a target cell type 1714 compared to non-target cell types 1716.

However, the two-timepoint method illustrated with reference to FIGS. 16-17 may ignore complex dynamics of cell trajectories, and result in less-optimal outcomes such as cell culture 1718. For example, the dynamics of the particular cell line may be different than the mean trajectory in previous observations. Generally, it would be advantageous to track the trajectory of cells over the entire cell culture process, and dynamically adjust the trajectory using the available tools, which are further described herein.

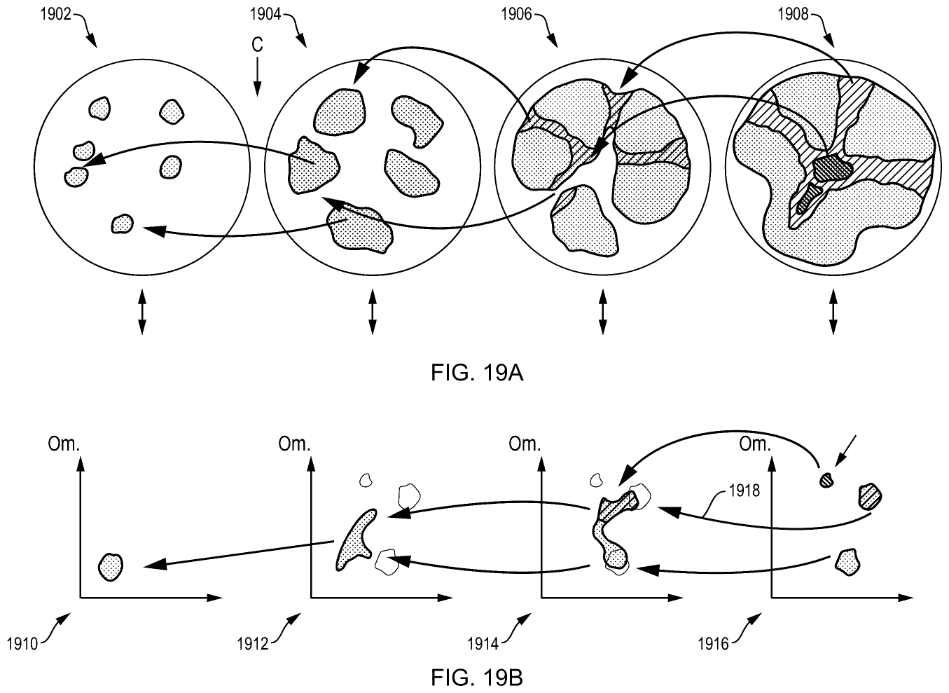

FIG. 18 is a diagram illustrating methods for tracing cell trajectories of a cell culture in space in accordance with various implementations. Multi-scale spatial conditions of the cell culture may be mapped into a latent space in which trajectories of successful and unsuccessful cell culture process outcomes may be traced, allowing for more holistic back-tracing of successful cell trajectories in a spatial context. FIG. 18 depicts a cell culture at four different timepoints 1802, 1804, 1806, and 1808 throughout a cell culture process. The cell culture system may utilize imaging to record the spatial configuration of the cell culture at different timepoints. At the end timepoint 1808, regions of the cell culture with the target cell type 1810 are mapped.

The cell culture system may back-trace cells of the target cell type 1810 backwards through timepoints 1806, 1804, and 1802, marking the likely regions where the cells of target cell type 1810 originated from (regions notated as 1812, 1814, and 1816 going backwards through time).

In cases in which individual cells are traced (e.g., using fluorescent-expressing cells or high-resolution label-free microscopy, and short time lapse intervals), the origin regions 1812, 1814, and 1816 may be precisely mapped. In cases in which label-free microscopy and/or longer time intervals are used (which allows for much broader data collection across many cell lines and runs), algorithms using unbalanced OT solutions may be used to trace likely origins and produce probabilistic maps for target cell origins. Unbalanced OT solvers contemplated herein may incorporate cell division probabilities as well as cell migration distance probabilities to weigh migration distances and proliferation in solutions to the timepoint-to-timepoint transport maps. These probabilities in turn may be driven by local cellular conditions, such as density and proximity to colony edges.

FIG. 18 also depicts spatial maps 1818, 1820, 1822, and 1824 corresponding to the cell culture at timepoints 1802, 1804, 1806, and 1808 respectively. The maps may represent a high-dimensional space that describes the multi-scale spatial conditions for a region of cells. Multi-scale conditions include spatial conditions on multiple scale levels, from a broad spatial level of the entire cell culture container down to a local spatial level that includes local cell morphology. The maps may preferably be in a reduced form that takes into account the repeated patterns observed in the cell culture. For example, if the cell culture is entirely circular cell colonies of varying diameter, a spatial configuration at the cell colony scale may be represented in a highly compact form in this latent space. The position of regions within cell colonies would represent the next lower scale, while at the next higher scale, inter-colony spacing may be represented. However, in most models an unsupervised approach may be used to reduce the spatial configuration into a latent space so the vector components in this space would not strictly correspond to specific visible features. A predictive model may be trained to maximally differentiate regions in this latent space that led to successful target cell populations from those that did not.

The latent space map 1824 of the end timepoint includes a representation of the target cell population 1826 within the overall spatial context. The target cell population 1826 may be traced back to prior timepoint maps 1822, 1820, and 1818 to identify the likely origin regions of the cells that end up as the target cell type 1810 (notated as regions 1828, 1830, 1832 going backwards in time). In this manner, a representation of successful spatial configurations in a reduced space is developed, and predictions of how the cell culture space evolves with time may be made. Further, the mapping of an image into this space may be inverted such that operations that would push more of the cell population within the latent space into the target cell type region may be translated into spatial operations within the cell culture. Then, a CRT may be used to shape the cell culture accordingly to manipulate the cell culture towards a trajectory that results in the target cell type.

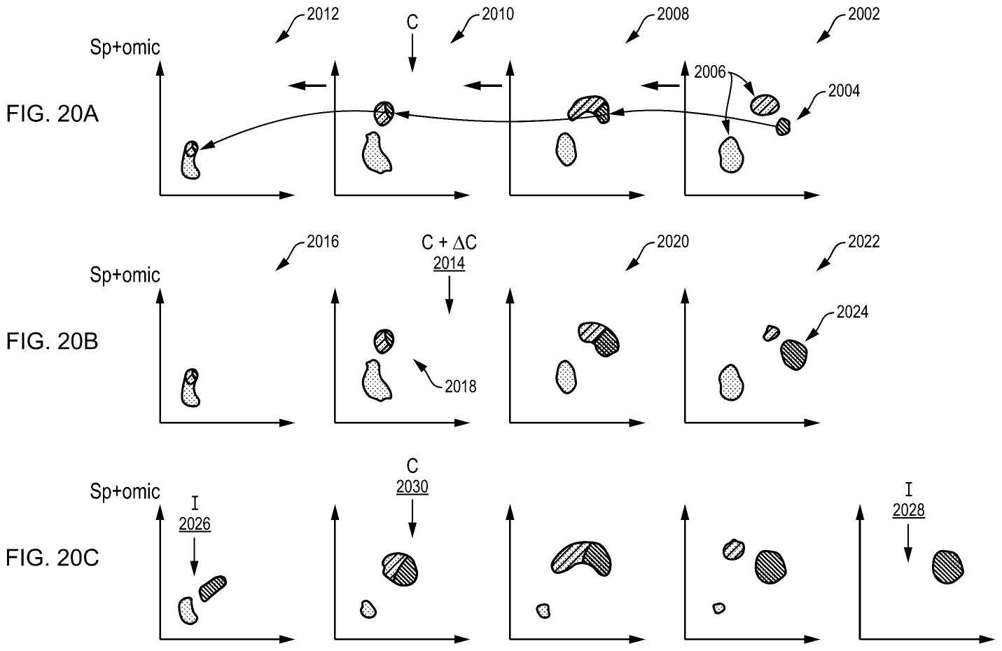

FIGS. 19A-B are diagrams illustrating parallel back-tracing of cell trajectories in both the spatial and -omic domains in accordance with various implementations. The back-tracing in both domains may be linked together such that causal links may be established between the -omic and spatial trajectories. FIG. 19A depicts a cell culture process history in which an initial cell culture state 1902 is transformed into a final cell culture state 1908 through intermediate cell culture states 1904, 1906. Using the methods disclosed herein, different phenotypic regions may then be traced backwards. For example, an OT-based solution may be applied to back-trace spatial paths of different phenotype outcomes.

FIG. 19B depicts a parallel (virtual) back-tracing in the -omic space corresponding to the spatial time series in FIG. 19B. The -omic maps 1910, 1912, 1914, and 1916 correspond to the cell culture states 1902, 1904, 1906, and 1908 respectively. The -omic map 1916 corresponding the final timepoint of the cell culture process may either be a true -omic map resulting from an assay (which is typically destructive, and therefore only usable in training runs), or a virtual -omic map that is generated from label-free or lightly-labeled image of the cell culture state 1908. For example, the -omic distribution may be generated via a pre-trained deep learning model that has been trained to label two or more phenotypes based on single timepoint or time series images. In other implementations, “lightly labeled” live cells (e.g., cells engineered to express fluorescent proteins, or using live cell labels) may be used to generate the -omic map. Based on prior -omic trajectory mapping, a back-tracing method 1918 may be used to reconstruct likely -omic maps for previous timepoints corresponding to the spatial (imaging-based) measurements in FIG. 19A. Thus -omic map 1916 may be used to reconstruct -omic maps 1910, 1912, and 1914 at earlier timepoints using back-tracing. In addition, regions within the -omic map 1916 representing the target cell type may be back-traced from the final timepoint to earlier timepoints on the -omic maps 1910, 1912, and 1914.

In this manner, correspondences between spatial conditions over time (trajectories) and -omic trajectories may be established. As a result, a model may be trained to predict -omic maps from spatial maps or series of spatial maps. This model may in turn be used to tune global conditions applied to the cell culture dynamically according to the progression of cells along the calculated -omic trajectory, in response to different run-to-run conditions, seeding conditions, cell line-to-cell line variations, etc. Moreover, this model may be used to iteratively compute a cell culture process intervention involving cell proliferation and selective cell removal by CRT that will likely shift the -omic outcome of the cell culture process. The model may forward-predict the effects of proliferation and different cell operations (e.g., cell removal, cell translation, density management, colony splitting) on the spatial trajectory (growth pattern) and the -omic trajectory simultaneously, and therefore determine a series of interventions on the cell culture process that will likely guide cell cultures toward a high yield output of cells of a target cell type.

In a joint model, spatial configuration may be used as a weight for -omic trajectory predictions. For example, the model may determine that certain -omic transitions are made with high probability when cells are at the edge of a cell colony, while cells at the dense center of a colony may have a different set of trajectory probabilities, while cells in small clusters may have a high probability trajectory towards termination. Likewise, the predicted -omic state of a spatial region may be weighted when making spatial trajectory predictions: different phenotypes will have different proliferation rates, density, motility, shape factors in resulting structures. Thus the joint backtracking-based measurement and predictive capabilities allow joint optimization of spatial and -omic trajectories using global conditions, timing of operations, and use of selective cell removal via a CRT.

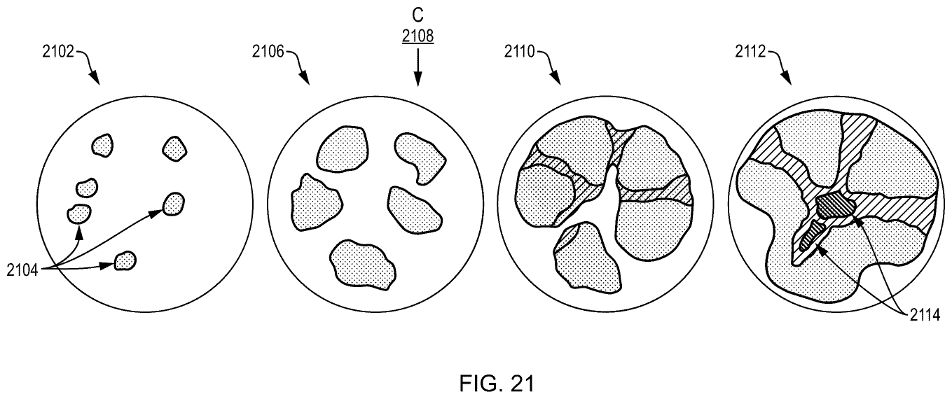

FIGS. 20A-C are diagrams illustrating methods for mapping -omic and spatial features of a cell culture process into a single latent space to train predictive models in accordance with various implementations. The predictive model may be trained by back-tracing in the combined -omic and spatial space to predict the effects of local spatial interventions and global conditions. This may enable holistic optimization of cell culture processes in a manner that captures the interrelationships between -omics and spatial conditions, and allows optimization of cell culture process yields, purity, functionality, and quantities.

FIG. 20A depicts a 2-dimensional map 2002 of a multidimensional joint space that represents both the spatial state and -omic state of a cell population at a final timepoint. The joint map 2002 distinguishes regions of the target cell population 2004 and non-target cell population 2006, which may be back-traced through the cell culture process to earlier timepoints, represented by maps 2008, 2010, and 2012. The combined space of the map 2002 may be constructed from parallel measurements, prediction, and tracking as described with reference to FIGS. 19A-B. Back-tracing cell trajectories in the spatial-omic space of maps 2002, 2008, 2010, and 2012 may be performed using OT algorithms based on as little as sparse timepoint label-free images, in which at least at the final timepoint a model has been used to assign -omic profiles to spatial regions based on these label-free images. A behavioral model predicting joint spatial-omic trajectories may be continuously updated even in production runs that output cells for clinical transplantation. In training-only runs to build the predictive models, fluorescently-labeled cell cultures and destructive assays may be used to collect spatial-omic data.

FIG. 20B depicts an example of using a predictive model trained on the spatial-omic data generated in FIG. 20A to control cell trajectories of future cell culture process runs. This can be done by, for example, modulating global conditions “C+ΔC” 2014 in response to a specific cell culture condition and trajectory. FIG. 20B depicts spatial-omic maps 2016, 2018, 2020, and 2022 at different timepoints of the cell culture process, progressing in time from left to right. The cell culture may be initiated to the same starting conditions as the cell culture depicted in FIG. 20A (i.e., the spatial-omic maps 2012 and 2016 may be almost identical). Example of global condition modulations may include, for example, varying the timing of a change in growth factors based on the spatial-omic trajectory (e.g., the mean size of colonies, the shape factor of colonies, and the proliferation rate of colonies which are linked to -omic features). By applying the modulated global condition 2014, the trajectory of the cell culture may result in an enriched, larger target cell population 2024 as compared to the unmodulated cell culture process.

FIG. 20C depicts another example of using a predictive model trained on the spatial-omic data generated in FIG. 20A to control cell trajectories of future cell culture process runs. In this example, cell trajectories may be altered using one or more spatial interventions “I” 2026, 2028 at different timepoints of the cell culture process. The spatial interventions may be performed, for example, by a CRT to selectively remove cells or perform other cell operations as disclosed herein. The earlier spatial intervention 2026 may be used, for example, to alter the spatial-omic trajectory towards a trajectory yielding a higher proportion of target cells. The later spatial intervention 2028 may be used, for example, to remove-target cells based on predictions of cell phenotype built through the entire tracking process. Global conditions “C” 2030 may be applied at a timepoint between the spatial interventions 2026, 2028. Generally, combinations of modulations to global conditions, spatial interventions (which may be applied dynamically), and other alterations to the cell culture environment may be used to alter the cell culture's spatial-omic trajectory based on determinations by the predictive model.

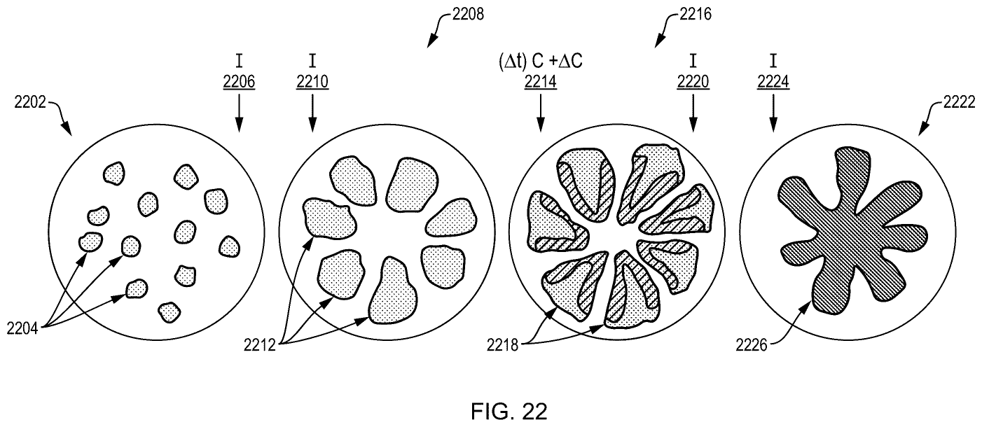

FIG. 21 is a diagram of a differentiation cell culture process prior to optimization by a predictive model in accordance with various implementations. The differentiation process begins with an initial cell culture state 2102 comprising a plurality of cell colonies 2104. The cell colonies proliferate as time progresses into intermediate cell culture state 2106, and then global conditions “C” 2108 are applied to the cell culture (e.g., adding a set of transcription factors). The cell culture proliferates further and begins to differentiate in certain regions, resulting in differentiated intermediate cell culture state 2110. At the end of the differentiation process, the final cell culture state 2112 contains target differentiated cells 2114 and at least two other non-target cell types, with the target cell type making up a low proportion of the overall cell population.

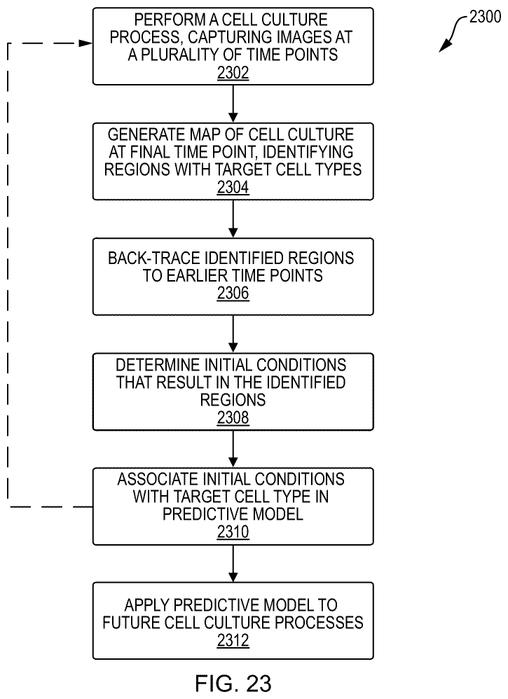

FIG. 22 is a diagram of a differentiation cell culture process after optimization by a predictive model in accordance with various implementations. The differentiation process begins with an initial cell culture state 2202 comprising a plurality of cell colonies 2204. The plurality of cell colonies 2204 that are seeded may be increased as compared to the cell culture process depicted in FIG. 21 because a cell removal tool may be used to shape the cell culture into an optimal state by subtraction at later timepoints. A first spatial intervention 2206 (denoted “I”) by a CRT may be used to selectively remove cell colonies to achieve consistent spacing and selectively down-size some cell colonies to achieve a consistent colony size. The first spatial intervention 2206 may further include patterning an extracellular matrix (ECM) on the surface of the cell culture container to slow down or stop the proliferation of cells in certain directions.

After initial colony proliferation into cell culture state 2208, a second spatial intervention “I” 2210 may be used to shape the merging cell colonies 2212. In this example, a back-tracing model may have determined that cell regions of a certain minimum size, with gaps of a certain distance, are ideal for promoting an optimal spatial-omic trajectory. Next, a modified set of global conditions “C+ΔC” 2214 (for example, introducing new transcription factors) may be introduced at a modified timepoint (with time differential Δt from the original timing as depicted in FIG. 21). The modifications to the global conditions and timing may be driven from real-time spatial-omic state and trajectory information gathered by the predictive model. At a later timepoint, the cell culture state 2216 may include uniform cell islands 2218 that exhibit the start of differentiation by some of the cells (shown by cross-hatching). A third spatial intervention “I” 2220 may be computed by the predictive model based on cell coverage and proliferation rate to tune the moment of colony-colony collision. At a later timepoint when the cell culture reaches cell culture state 2222, a fourth spatial intervention “I” 2224 may be performed to remove non-target cell types, leaving only target cell type 2226, albeit with a much larger final cell population than in the pre-optimized process shown in FIG. 21.

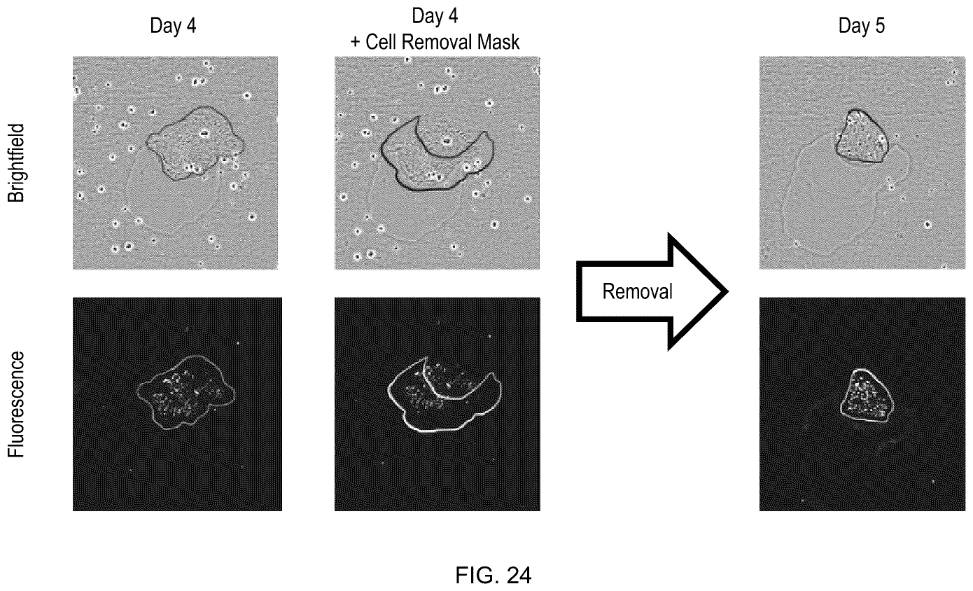

FIG. 23 is a flow chart of a method 2300 of training and using a predictive model based on cell culture process trajectories in accordance with various implementations. The method 2300 may be performed by a cell culture system that includes one or more cell culture containers, an imaging subsystem capable of capturing images of cell culture processes in the one or more cell culture containers, a CRT, and a computing subsystem supporting a predictive model.

In block 2302, the cell culture system may perform a cell culture process on a cell culture in a cell culture container. The cell culture process may be, for example, reprogramming, expansion, differentiation, or rejuvenation. The cell culture system may be configured to capture images of the cell culture at a plurality of timepoints throughout the cell culture process, from an initial timepoint to a final timepoint.

In block 2304, the cell culture system may be configured to generate a map of the cell culture at the final timepoint, the map identifying regions of the cell culture that contain one or more target cell types. Different cell culture processes have different output target cell types, and for example may include successfully differentiated cells, successfully reprogrammed clonal iPSCs, or successfully rejuvenated cells. The map may be an -omic trajectory map, a spatial trajectory map, or a combination of both.

In block 2306, the cell culture system may be configured to back-trace the identified regions containing the target cell type to corresponding maps in earlier timepoints. This can be done by generating corresponding maps from the images taken at the plurality of timepoints, and tracing the cells back through time by finding the corresponding regions in the maps.

In block 2308, the cell culture system may be configured to determine the initial cell culture conditions (e.g., spatial configuration) that result in the identified regions of the target cell type. In block 2310, the cell culture system may associate the initial conditions with the target cell type in the predictive model, indicating that if the initial conditions can be replicated in the cell culture, then more of the cell culture will transition to the target cell type by the end of the cell culture process. Blocks 2302-2310 may be iteratively performed to improve the performance of the predictive model.

Once the predictive model is sufficiently trained, in block 2312 the cell culture system may be configured to optimize future cell culture process runs utilizing the predictive model. For example, the cell culture system may utilize a CRT to selectively remove cells or ECM to alter the initial spatial conditions of the cell culture or to manipulate the cell culture during the cell culture process, or to make changes to global conditions to the cell culture. These interventions are intended to push the cell culture towards the cell trajectory leading to the target cell type. Thus in this manner, a cell culture system may utilize the method 2300 to produce high quality, high quantity output cell products in biomanufacturing applications.

The various implementations disclosed herein may enable a variety of cell culture optimization methods. For example, some cell culture processes involve the culture of initially heterogeneous cell populations. In such cases, it is often desirable to isolate clonal subpopulations originating from a single proliferative cell. Such a clonalization process may be achieved via cell operations disclosed herein (e.g., iterative removal of a portion of a heterogeneous cell population to encourage growth of a clonal subsection of the cell population). The clonal population to isolate may have certain preferred inheritable traits that distinguish the subpopulation from surrounding cells. Such traits may include the enhanced potential to transform into a particular transcriptomic state, the presence of a genetic modification, the presence of an inheritable fluorescent label, or certain morphological features such as cell size or texture. In such cases, methods of imaging-based detection of desired clonal subpopulations along with selective cell removal by a CRT as disclosed herein may be used to identify and isolate a clonal subpopulation from surrounding undesired cells.

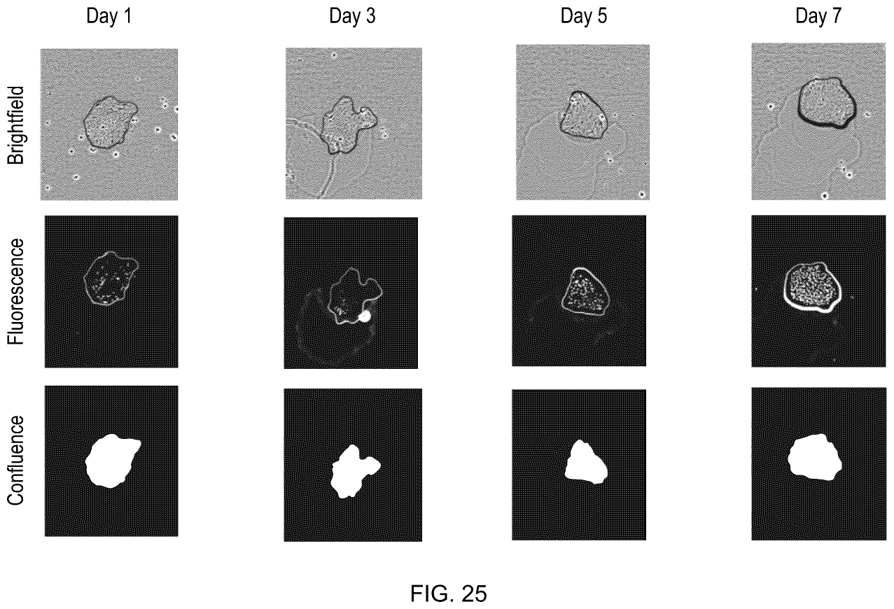

One example application of clonalization is the isolation of cells containing a genetically integrated fluorescent tag, such as a nuclear localized fluorescent protein. Single cells containing this tag may be observed in a mixed population via fluorescence microscopy over time. This may enable the tracking of a single cell as it proliferates to form a clonal neighborhood. All non-fluorescent cells surrounding this neighborhood may then be removed using a CRT, and the isolation of the clonal fluorescent subpopulation may be verified via post-removal fluorescence imaging. An example of approach as applied to human iPSCs in a multi-day cell culture process is shown in FIG. 24.

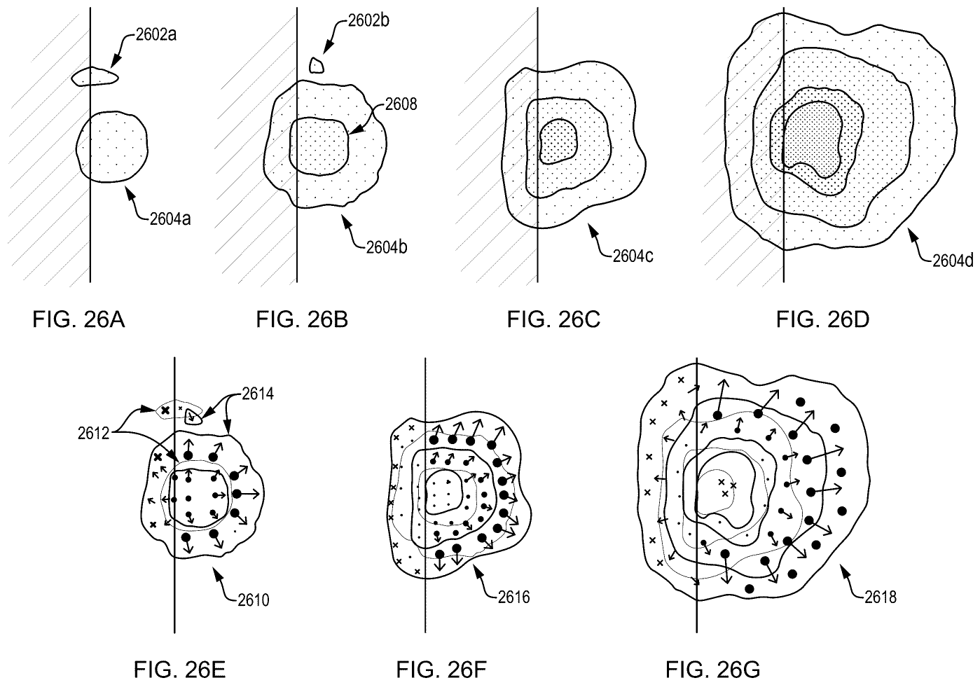

In cases in which the target cell population is sparse, highly heterogeneously mixed with unwanted cells, or not detectable via imaging, the process for clonalization described herein may be repeated for several cycles. Repeated cell growth and removal cycles progressively clonalizes the cell population. FIG. 25 illustrates an example of clonalization of a homogeneously mixed population of fluorescent and non-fluorescent iPSCs using the methods disclosed herein, wherein the cell population converges towards a fully fluorescent (i.e., clonal) population via repeated cell growth & removal cycles.

In some cases, a target cell population may be identified via cell modulation approaches, such as those disclosed herein. For example, in some cases of genetic editing of cells an antibiotic resistance cassette may be introduced, causing edited cells to display enhanced survival in the presence of a specific antibiotic molecule. A heterogeneous population of cells containing both antibiotic-resistant and non-resistant cells may be treated with a low dose of antibiotic and cellular response may be identified via light microscopy. Cells displaying markers of poor cell health, such as membrane blebbing or cell shrinkage, may be discerned from healthy cells by an operator, a computational image analysis method, a trained image-based machine learning model, or combinations therein. This may be followed by the isolation of a local sub-population of healthy cells via a CRT, enabling the clonalization of the subpopulation.

Another example of clonalization target identification via cell culture modulation involves the detection of cellular responses to cellular perturbations. For example, cells may be treated with a chemical compound, or a physical stimulus such as an electric or fluid flow field. A dynamic readout from the stimulated cells, such as a morphological change or the response of a fluorescent or other optically observable reporter molecule, may be measured via time-resolved imaging techniques. Features of this measurement may then be interpreted by an operator or a predictive model to identify cell neighborhoods with desired response dynamics, and these cells may be isolated from neighboring cells via a CRT. Such an approach may be used in the high-throughput screening of a library of optical reporters, in which the goal is to identify reporters with a target set of kinetic or intensity properties.

In certain cases, it might be desirable to isolate cell subpopulations originating from a specific location within the cell culture container at the beginning of a cell culture process that involves extensive cell proliferation and motility. In such cases, the imaging and cell isolation methods disclosed herein may be used to infer cellular flows driven by proliferation and movement based on a time series of microscopy images. Specifically, predictive models may be designed to take as input a defined cell neighborhood in a first timepoint of a time series, and return predicted spatial probability maps of cell distribution at later timepoints. This may enable the prediction of descendants of a defined cell neighborhood at a terminal timepoint, when a CRT can be used to isolate these predicted descendant neighborhoods for clonal isolation.

### Systems and Methods for Measuring Cell Culture Dynamics

Cell culture processes (such as expansion, purification/enrichment, reprogramming, rejuvenation (including partial reprogramming), differentiation, and transdifferentiation) may benefit from more accurate, granular information from label-free in situ measurements. Even for adherent cell cultures with primarily monolayers of cells, current measurement methods are limited, often relying on single time point morphological characterization of cells. In the past several years, advances have been made in the use of deep learning models to more accurately and consistently classify cells via morphological features. However, there is still a very large gap in the ability to measure cell state and trajectory from label-free timelapse imaging alone.

The state of the art, as well as the disclosures incorporated by reference, may benefit from more precise estimation of cell state and trajectory based on imaging, mapping, and computed tracking. Such estimates may be used to better control cell culture trajectory, efficiency, purity, yield, and ultimately applicability to human health and longevity. The measurement of cell dynamics in cell culture processes is non-trivial. Even with labeled methods (e.g., nuclear fluorescence), the task of tracing cell dynamics (e.g., motion, division, death) requires high frequency imaging, which can be disruptive or even destructive to cells, and sophisticated algorithms to trace individual cells and lineages. Thus there is the need in the art for improved methods of collecting large-volume cell information during a cell culture process in manner that does not disrupt the cell culture process or damage the cells.

The various implementations disclosed herein are directed towards measurement of cell culture dynamics. In an example implementation, systems and methods disclosed herein for measuring cell culture dynamics includes imaging a cell culture using an imaging system during at least two timepoints. The imaging may be label-free (such as brightfield imaging), the imaging timepoints may be too far apart to track individual cells, and the imaging resolution may be too low to identify individual cells. The systems and methods further include spatially mapping the cell culture at the at least two timepoints. These spatial maps may include, for example, cell presence maps, cell density maps, inferred protein expression maps, and cell texture/morphology maps (not necessarily at the cell level). The systems and methods further include computing an optimal transport plan which explains the evolution of the cell map from the first timepoint to the second timepoint using a combination of cell motion, cell division, cell death and/or cell state transitions. The systems and methods further include estimating the dynamics (e.g., motion, division, death, and or state change versus local and/or global conditions) of at least one cell population in the cell culture (e.g., the cell culture dynamic parameters) based on the transport plan.

In some implementations, the dynamics required for the transport plan are compared to the dynamics of one or more known cell types or states to classify cells or cell regions by likely cell state (“good” or “defective,” or type A or type B, etc.). This classification may be used to select certain regions of cells and deselect others to enrich the overall cell culture for the desired output cell type/state, through the use of a cell removal tool (CRT) to remove unwanted cells and cell regions. The classification may include deviations from a desired cell type/state, including but not limited to differentiation, karyotypic abnormalities, epigenetic changes, mutations, etc., as well as predictions of future states or impacts on the state of cells of cell regions.

In some implementations, cell state or type transitions may be incorporated into the optimal transport plan between timepoints of a cell population. For example, the transport plan may be used to trace back the lifecycle of different cell types/states at the endpoint of a training process to deduce optimal conditions (e.g., global, local, spatial) to produce optimal cell trajectories.

In some implementations, the dynamics of a cell population may be measured to estimate cell dynamic parameters. In some implementations, a map of the cell population and estimated parameters may be used to predict a future state. In some implementations, one or more potential actions on the cell culture may be evaluated as a way to change the trajectory of a cell culture. In some implementations. The one or more potential actions may include, for example, selective removal of cells using a CRT, selective removal of extracellular matrix (ECM) using a CRT, a change in media components, a change in gas concentrations, a change in pH, a change in temperature, a change in timing of media changes, harvesting all or some of the cells from the cell culture, or changes in other environmental parameters. The action plan may be generated using a simulation based on the cell culture parameters, or using a reinforcement learning model that has been trained on prior runs of the cell culture process, and/or prior runs of simulations.

Methods of explaining spatial evolution of the cell population over time may include, but are not limited to, diffusion modeling such as modeling by the Fisher-Kolmogorov-Petrovsky-Piskunov equation, or solutions to optimal transport. Global or local cell region parameters may be estimated by a number of methods, including but not limited to those described in Liu, Ye et. al., “Parameter identifiability and model selection for partial differential equation model of cell invasion,” J. R. Soc. Interface 21: 20230607 (2024) and Puliafito, Alberto et. al., “Collective and single cell behavior in epithelial cell contact inhibition,” PNAS, vol. 109, no. 3, 739-744 (January 2012), each of which is hereby incorporated by reference in their entireties.

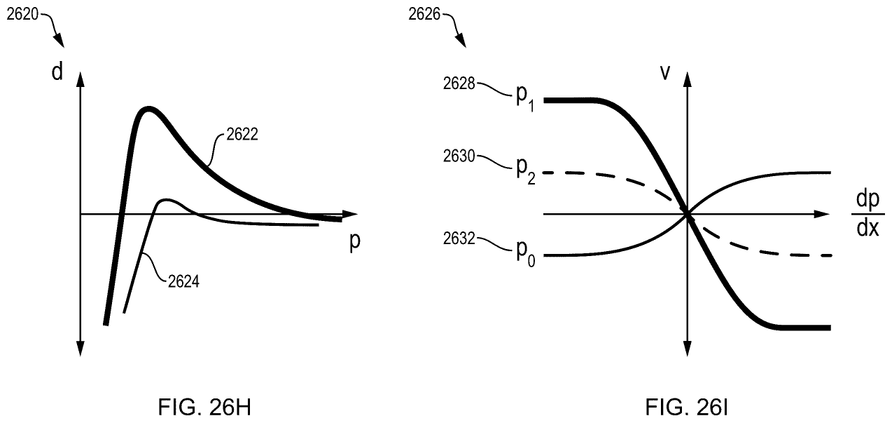

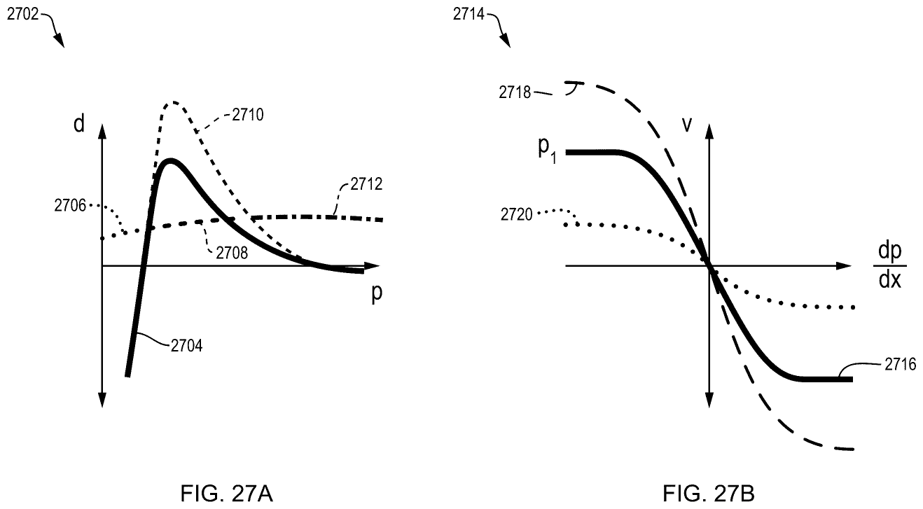

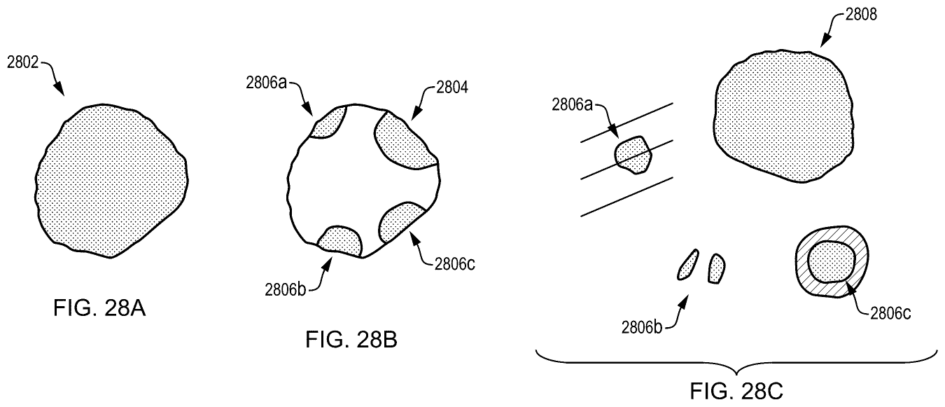

FIGS. 26A-D are diagrams illustrating a cell population at four different timepoints in accordance with various implementations. The cell population may be imaged at each illustrated timepoint. The various implementations disclosed herein may be used to map the cell dynamics that link each timepoint to the next. The imaging may be performed using label-free imaging. Images may be converted into maps of cells, including but not limited to maps of cell confluence, cell density, cell phenotype, and/or latent space representations of local cell characteristics generated by deep learning and other models. With reference to the example shown in FIGS. 26A-D, the cell population is illustrated using cell density maps. The systems and methods disclosed herein are designed to connect timepoints that are too far apart temporally to apply individual cell tracing, which has typically been used (in addition to fluorescent labeling or expression) for cell dynamics studies in the prior art.

FIG. 26A illustrates a cell population a first timepoint, the cell population including a small cell cluster 2602a and a large cell cluster 2604a. Both cell clusters 2602a, 2604a are partially located in a cell culture surface region 2606 in which an ECM has been partially or entirely removed. For example, this may happen due to scanning of a pulsed laser over a cell culture surface that includes a laser-absorptive coating, which results in rapid heating and/or bubble expansion and collapse in the targeted region and removal of cells and the ECM in that region.

FIG. 26B illustrates the cell population at a second timepoint. The small cell cluster 2602b has contracted, or lost cells, by the second timepoint, particularly in the region 2606 lacking ECM. The small cell cluster 2602b may also have moved closer to the large cell cluster 2604b. The large cell cluster 2604b has expanded due to cell proliferation and motion, and the proliferation has further resulted in a higher-density region 2608 within the large cell cluster 2604b. For the purposes of drawing interpretation, lines within a cell cluster are meant to show iso-density lines, with low density regions at the perimeter of the cell cluster and progressively higher densities within the cell cluster.

FIG. 26C illustrates the cell population at third timepoint. Here, the large cell cluster 2604c has expanded and subsumed the remaining piece of the small cell cluster. The large cell cluster 2604c has increasing cell density towards the center of the cluster, as shown by the iso-density lines. FIG. 26D illustrates the cell population at a fourth timepoint, at which the large cell cluster 2604d has further proliferated, grown in area, grown in cell count, and grown denser in its center.

FIGS. 26E-G illustrate cell dynamics maps generated from images of the cell populations at the timepoints illustrated in FIGS. 26A-D in accordance with various implementations. The cell dynamics maps may allow for reconstruction of cell trajectories and dynamics to link the timepoints.

FIG. 26E illustrates a transport map 2610 mapping the cell population at the first timepoint to the cell population at the second timepoint. The transport map 2610 may include multiple layers of scalar and vector values that connect the cell density map of the cell population at the first timepoint (FIG. 26A) to the cell density map at the second timepoint (FIG. 26B). In some implementations, the transport map 2610 may be constructed using cell density information from multiple other timepoints between the first and second timepoints. Outlines 2612 show the positions of the small and large cell clusters 2602a, 2604a at the first timepoint. Outlines 2614 show the positions of the small and large cell clusters 2602b, 2604b at the first timepoint. The transport map 2610 shows the best estimation of cell growth and translation from the first timepoint to the second timepoint, in which dots indicate cell proliferation/division (with larger diameter dots corresponding to higher division rate), “X” marks indicate net cell death, and arrows indicating net cell motion.

In this manner, a combination of cell division, death, and motion are synthesized to optimally explain the trajectory of the cell population from the first timepoint to the second timepoint. This transport map 2610 may be computed via a range of models, such as solutions to unbalanced optimal transport problems (including but not limited to Sinkhorn algorithms, entropic regularization schemes, non-negative penalized algorithms, scaling algorithms, unbalanced gradient flows, unbalanced barycenters, unbalanced minibatch optimal transport, unbalanced low-rank optimal transport solvers, iterative evaluation of partial differential equation-based models, and deep learning such as generative adversarial network (GAN) approaches to optimal transport).

FIG. 26F illustrates a transport map 2616 mapping the cell population at the second timepoint (FIG. 26B) to the cell population at the third timepoint (FIG. 26C). Again, a series of sources/sinks and vectors (represented by dots, X's, and arrows) are calculated to represent the transport from one timepoint to another. In general, these transport maps may in fact represent multiple transport maps between two timepoints, which interpolate the cell dynamics at multiple points (i.e., a series of N transport steps may be used to detail the dynamics between two timepoints, to enable more accurate tracing of final cellular origins within the initial population and to capture intra-timepoint cellular dynamics).

FIG. 26G illustrates a transport map 2618 mapping the cell population at the third timepoint (FIG. 26C) to the cell population at the fourth timepoint (FIG. 26D). The multi-dimensional vector maps (dots and arrows) within the transport map 2618 represent the trajectories of cells as they proliferate, move, and/or die off. Specific regions of cell death are indicated by X's. For example, cell death in the transport map 2618 are in regions (1) where the lack of ECM results in bad cell proliferation and health, resulting in a net cell “sink” (death); and (2) a region in the center of the cell cluster where cell density is too high, resulting in cell death or detachment during washing/media change operations that are represented by a net cell loss.

FIGS. 26H-I illustrate examples of aggregate cell dynamics for the cell population of FIGS. 26A-D that may be generated from the transport maps shown in FIGS. 26E-G in accordance with various implementations. FIG. 26H illustrates an example of a graph 2620 of cell proliferation/death as a function of cell density, as compiled from the transport maps show in FIGS. 26E-G. Plot 2620 shows net growth or division rate per time (vertical axis “d”) versus local cell density (horizontal axis ρ). A first plot of cell data 2622 shows the proliferation rate as a function of density for cells on ECM, for example. The characteristics of this plot 2622 are typical of pluripotent cells such as embryonic stem cells (ESCs) or induced pluripotent stem cells (iPSCs). At very low density, these cells do not survive without additives (such as ROCKi) to the media, resulting in a negative growth rate (as indicated by the shrinkage of very small clusters). At medium-low densities, the cells proliferate rapidly, for example dividing every 18 hours. At higher densities, contact inhibition slows the proliferation rate significantly. At the highest densities, the colonies may show a negative growth rate due to cell death from overcrowding, or detachment of cells from the 2D colony during washing or media changes.

A second plot 2624 shows the same data, extracted from regions where the cells are growing on surfaces depleted of ECM, for example as the result of spatially-selective cell removal via a CRT, ECM patterning, ECM non-uniformities within the cell culture container, and/or selective ECM removal by a CRT or other tool. The low-ECM or no-ECM proliferation curve generally shows lower or negative net growth, with a higher cell density needed to sustain itself, and only a narrow range of densities at which cells can survive, mostly by cell-to-cell contact.

FIG. 26I illustrates another graph 2626 generated from the transport maps shown in FIGS. 26E-G. The graph 2626 shows plots, at various local cell densities, of net cell velocity (vertical axis “v”) as a function of density gradient (horizontal axis dp/dx). For example, a first trace 2628 shows cell motion dynamics at medium-low density ρ1. This trace indicates cells have a net motion away from more dense regions into lower-density or empty regions, thus the “positive” velocity into a negative gradient, and vice-versa. The velocity saturates at a maximum, and at a zero density gradient, no net motion is observed. A second trace 2630 shows the same cell motion dynamics at a high density ρ2. The same overall pattern is observed, but the net velocities are lower due to cell crowding. A final data trace 2632 shows behavior at a very low density ρ0, at which the velocity profile is inverted for this cell type. At very low densities, the cells attempt to consolidate, or move towards regions of higher cell density. This behavior is typical of pluripotent cells, in which cells seeded in single-cell cultures consolidate and cells seeded as clusters tend to initially contract in area /rise in density.

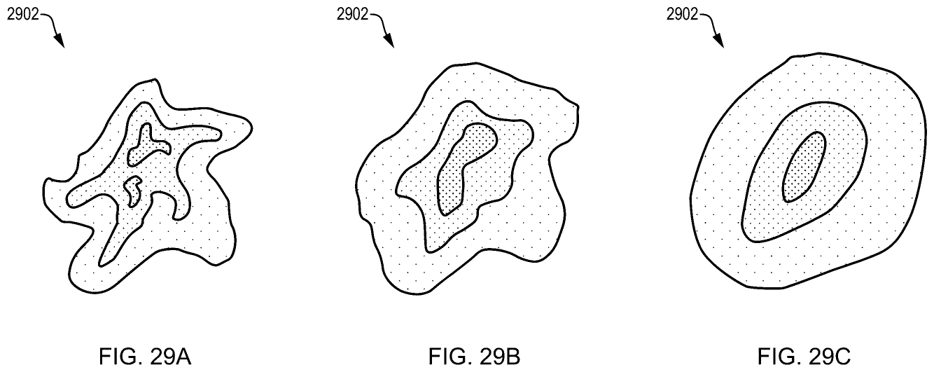

FIGS. 27A-B are cell dynamics graphs for comparing observed cell dynamics to baseline cell dynamics behavior in accordance with various implementations. The baseline cell dynamics behavior may be compiled from, for example, multiple cell cultures and processes in which the resulting cells passed all quality control and/or functional tests. The conditions under which cell dynamics are evaluated may vary based on other factors, such as local cell culture conditions, location within a growth container, stage of a cell process, patient or clone characteristics, etc. For example, one range of dynamics may be considered “normal” for management and proliferation of pluripotent cells, and deviations from normal pluripotent cell dynamics may be seen as signs of genomic damage or lowered pluripotency. However, during subsequent differentiation of these pluripotent cells, certain deviations from the pluripotent dynamics may in fact signal successful initial differentiation.

FIG. 27A illustrates a graph 2702 of net cell proliferation versus cell density, similar to the graph 2620 shown in FIG. 26H. The graph 2702 plots net growth or division rate per time (vertical axis “d”) versus local cell density (horizontal axis p). A solid trace 2704 shows, for example, the “normal” dynamics of a class of pluripotent cells, with death at very low densities, a peak growth rate at a medium-low density, and slower growth rates at higher densities due to contact inhibition. Additional curves/segments are shown that represent departures from this normal dynamic, which can result from a range of effects. For example, cells may be observed that are healthy and even proliferating at very low density as indicated by trace 2706. This may result, for example, from spontaneous differentiation in pluripotent cells, in which the survival and proliferation of cells at low densities around the edges of cell colonies is often a sign of spontaneous differentiation. Possible responses to this situation may include one or more of: logging spontaneous differentiation in the history of the cell population/clone, removing the spontaneously differentiated cells from the cell culture using a CRT, and/or removing the cell colony of origin entirely. The survival of pluripotent cells at low densities may also indicate karyotypic or genomic abnormalities, as evidenced by enhanced survival through cell passaging steps conferred by some such abnormalities. In an active cell differentiation process, the appearance of this dynamic may signal successful initial stages of differentiation.

Trace 2708 shows observed cell dynamics behavior at medium-low density, in which a lower proliferation rate may also indicate differentiation, as the cell cycle extends. It could also indicate only partial reprogramming of cells into pluripotency. In other cases, it may indicate some genetic damage or senescence in the cells. Another observed case, in which cell division is abnormally rapid, is indicated by trace 2710. Abnormally rapid cell division may indicate karyotypic abnormalities, among other issues, in the pluripotent cells. Finally, cell division that continues unabated at higher densities, shown by trace 2712, may indicate a range of issues that result in a lack of contact inhibition. Among the causes for this may be mutations in oncogenes, including but not limited to p53, Rb, PTEN, RAS, MYC, SRC; aneuploidies; and/or abnormal methylation or histone modifications. In many differentiation protocols, terminally differentiated cells should become post-mitotic and therefore cease proliferation entirely, so the expected behavior would be a flat line on the y-axis, and regions that show dynamics where proliferation persists may indicate a bad cell culture, or trigger removal of those regions by CRT. In such terminally differentiated cell cultures or tissues, the persistence of proliferation at higher densities may indicate tumorigenicity. Similar curves can be plotted for cells on low- or no-ECM regions.

FIG. 27B illustrates a graph 2714 of net cell velocity versus cell density gradient, similar to the graph 2626 shown in FIG. 26I. The graph 2714 plots net cell velocity (vertical axis “v”) as a function of density gradient (horizontal axis dp/dx). The graph 2714 includes a solid line plot 2716 indicating the expected behavior of cells, namely moving towards areas of lower density with some saturation in cell velocity. Two other plots, 2718 and 2720, illustrate deviations from expected behavior, all at the same local density ρ1. Plot 2718 shows a case in which faster than normal velocity is measured. In some cases, this velocity is so fast that cells cross over one another, indicating the ability to migrate over other cells. Such behavior may indicate differentiated cell types, including even semi-adherent cells. Conversely, plot 2720 illustrates low velocity across a wide range of density gradients and may indicate the inability to migrate, or the beginnings of a cell transformation (such as differentiation). In the extreme case, the curve may reverse, indicating that cells are compactifying, which may indicate differentiation or formation of a 3D structure from a 2D structure.

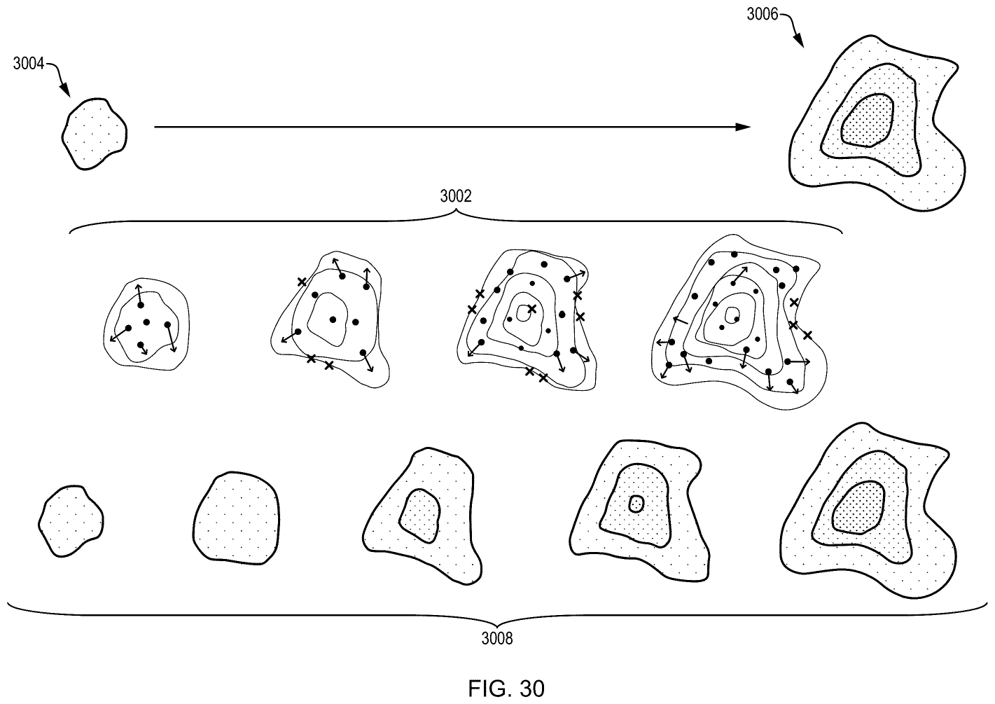

FIGS. 28A-C are diagrams illustrating splitting of test colonies from a primary cell colony in accordance with various implementations. The test colonies may be used to test cell dynamics under various conditions. FIG. 28A shows a primary cell colony 2802 in a cell culture container (e.g., a closed cassette). FIG. 28B shows the use of a CRT (e.g., a pulsed laser scanner) to remove the majority of the primary cell colony 2802, leaving a reduced primary cell colony 2804 and three test colonies 2806a-c. The reduced primary cell colony 2804 may have a healthy cell density after cell pruning and continue on for downstream processing (e.g., cell colony maintenance through a cell culture process). In some implementations, the test colonies 2806a-c may be created serially rather than simultaneously. The test colonies 2806a-c may be translated/manipulated by further actions of the CRT.

FIG. 28C shows the reduced primary cell colony 2804, which has been allowed to proliferate and grow, and the three test colonies 2806a-c, which may be used to measure cell dynamics under different conditions. For example, the first test colony 2806a may be moved into an area of the cell culture surface with depleted ECM to analysis cell dynamics under ECM-depleted conditions. The second test colony 2806b may be split into two smaller cell clusters to look at survival under low-density conditions, and/or motility of cells rejoining to form a larger cluster. The third test colony 2806c may be surrounded by a “moat” formed from complete ECM removal, to observe cell dynamics as cell density increases (and to what extent cells extend into the ECM-free moat). In general, the test colonies 2806a-c may be manipulated, translated, and exposed to various conditions to ascertain cell dynamics. For example, the tests may include variations in media components, temperature, flow rates, etc. Based on the dynamics observed in the test colonies 2806a-c, the state or quality of the reduced primary cell colony 2804 may be predicted or inferred.

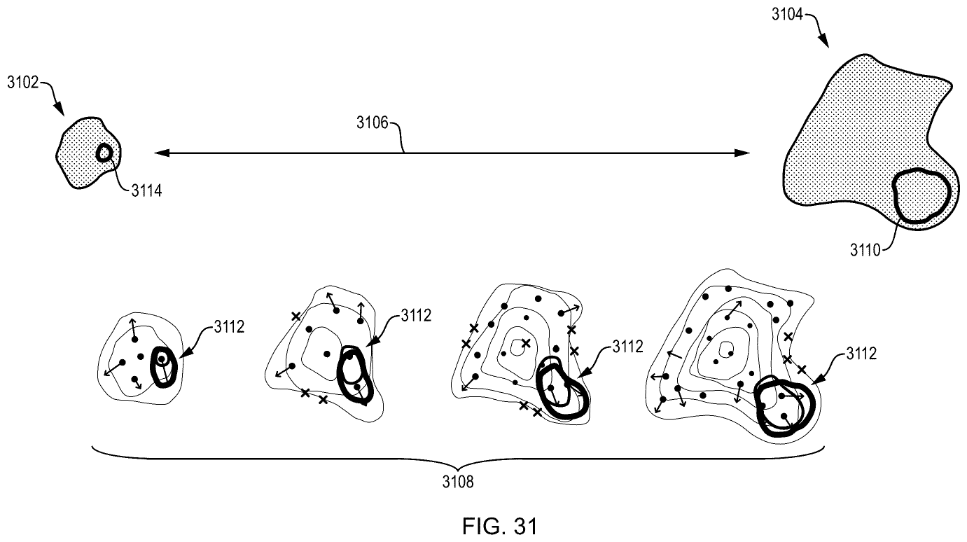

FIGS. 29A-C are diagrams illustrating the use of multiple scales of density measurement used to classify cell colony regions in accordance with various implementations. FIG. 29A shows a map of cell density of a cell colony 2902 at a low sigma smoothing (i.e., more differentiation between regions of different cell densities, resulting in more identified regions of different cell densities). The lines in the cell density map may be iso-density lines. FIG. 29B shows a map of cell density for the same cell colony 2902 and medium sigma smoothing. FIG. 29C shows a map of cell density for the same cell colony 2902 and high sigma smoothing (i.e., less differentiation between regions of different cell densities, resulting in fewer identified regions of different cell densities).

Cell structure behavior may be characterized and predicted at different scales, based on the different smoothing sigmas shown in FIGS. 29A-C. For example, cells in a small neighborhood of high density (identified in a low-sigma map such as FIG. 29A) in an overall low-density region (identified in a high-sigma map such as FIG. 29C) may have dynamic behaviors different from cells at the same small-neighborhood density within a larger structure of the same high density. The example discussed herein pertains to cell density, but density gradient and other properties may be evaluated at multiple scales as well. In some implementations, structures that span from local detail to the global may be mapped to a latent data space that captures this multi-scale representation. Such multi-scale representation is particularly useful when there are multiple signaling systems in play, e.g., cell-to-cell contact (or lack thereof), cell-to-cell chemical communication, local chemical signaling through media (or depletion of nutrients/buildup of waste products), and global chemical signaling. Similarly, there may be ECM patterning or gradients at various scales. Similarly, there may be flow, nutrient, waste, pH, gas concentrations that are container- and process-dependent acting at multiple scales.

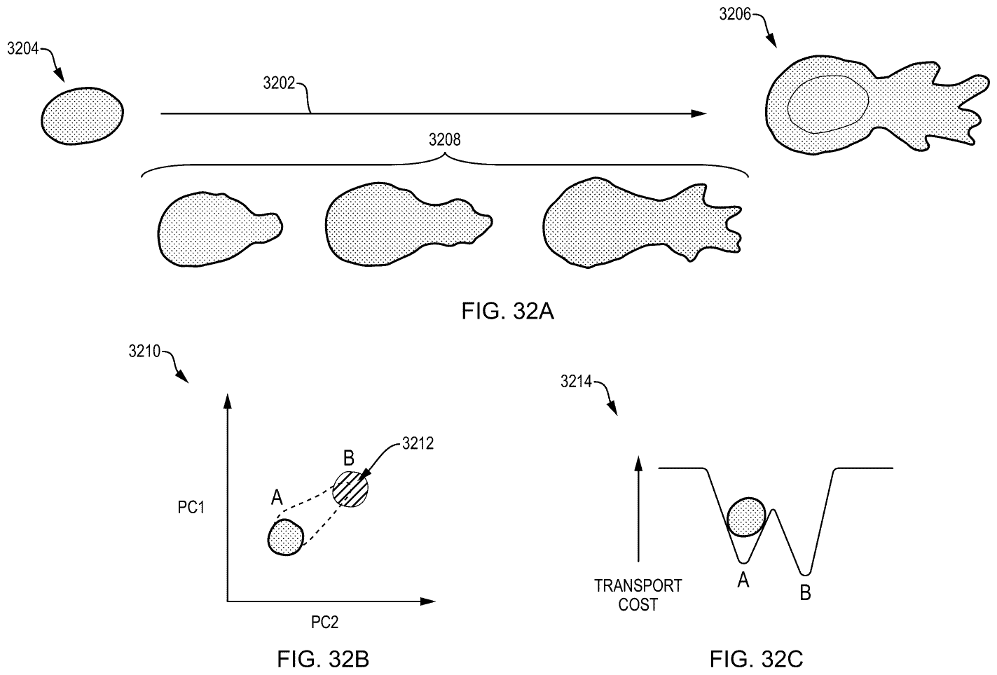

FIG. 30 is a diagram illustrating the reconstruction of a cell transport plan 3002 using data from two timepoints in accordance with various implementations. The transport plan 3002 may include not only cell motion, but may also include cell division (proliferation), cell death, and cell transformation (e.g., senescence, differentiation, reprogramming, rejuvenation, maturation, activation, gene editing, transfection, etc.). In the example shown in FIG. 30, the cells may all be of the same type/state as the population grows from a colony at a first timepoint (represented by colony density map 3004) to the same colony at a second timepoint (represented by colony density map 3006). The colony density maps 3004, 3006 indicate cell density as a series of iso-density lines (more dense towards the center of the colonies).

The optimal transport plan 3002, represented by a sequence of plans including cell proliferation (points), cell death (X's), and cell motion (arrows), is iteratively optimized to minimize transport cost. Transport cost may be calculated not only by distance, but may also be calculated by how far off the expected cell behavior each cell (or “unit,” which can include multiple cells) is from the optimal transport plan 3002. For example, net cell motion may be a function of local cell density (to simulate a “no-hopping” rule) as illustrated in FIG. 27A. If a proposed transport plan calls for motion significantly larger than the normal behavior, a cost is incurred in the proposed transport plan. Similarly, if the cell type is contact-inhibited, cell division will drop rapidly with local cell density. In such a case a proposed transport plan that calls for rapid cell division in high-density areas (or very little cell division in medium-low density areas) will incur a significant cost, pushing the optimizer towards a transport plan more in line with expected cell behavior.

In the process of calculating and optimizing a transport plan, the optimizer may calculate intermediate cell density maps 3008. These density maps 3008 may in turn be used to predict expected cell dynamics (e.g., motion, division, death) in different regions to complete and calculate the cost of the transport plan. Thus an iterative process may be set up wherein a transport plan creates a series of cell maps, which in turn are used to calculate the cost of the proposed transport plan. Maps may include not only cell confluence and density, but cell states, and the transitions between cell states may be included in the transport plans. Ultimately, the process may be used not only to describe a transport plan for a known cell type(s) or state(s), but also to characterize cell type(s)/state(s) as described herein.

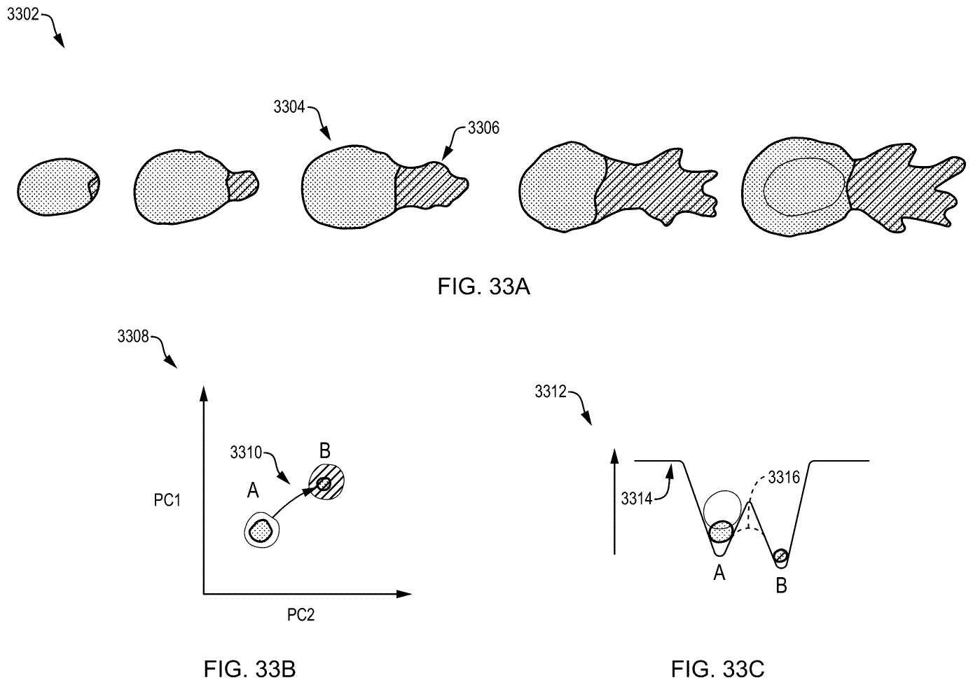

FIG. 31 is a diagram illustrating the use of transport plans to trace cellular origins and/or destinations in a cell culture process in accordance with various implementations. The transport plans may be built to match the dynamics of a particular cell type or state. In the example shown in FIG. 31, the cells in a cell colony may be traced from a first timepoint (represented by colony density map 3102) to the same colony at a second timepoint (represented by colony density map 3104). The tracing may be done forwards or backwards, as indicated by bidirectional line 3106.

An optimal transport plan 3108 (which may include cell motion, division, death, state changes, etc.) may be generated that explains the transformation of the cell colony at the timepoint to the cell population at a second timepoint. While the example shown in FIG. 31 are single cell colonies in two dimensions, the systems and methods described herein is applicable to all manners of arrangements of cells, including colonies, large sheets, mixed cell populations, and 3-dimensional cell structures. In this example, a region of interest 3110 is specified at the second timepoint. This region 3110 may be identified, for example, as having certain characteristics, or be identified as a potential representative region for creating a test colony or extracting material for in process analysis. Thus it may be desirable to trace back the origin of the region 3110. The methods described herein allows backward tracing of the region 3110 via the inversion of the optimal transport plan 3108 generated to transform the cell culture from the first timepoint to the second timepoint. By inverting cell motion, cell death, cell division, and cell state changes along the generated transport plan 3108, the region of interest may be traced back through intermediate states 3112. Thus the cell region of interest 3110 may be traced back to a corresponding region 3114 of the cell colony at the first timepoint.

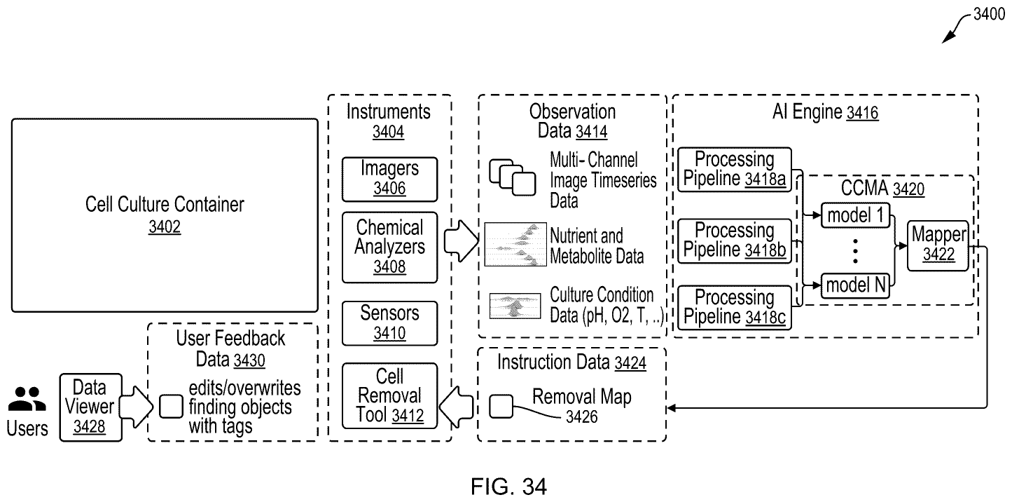

FIGS. 32A-C are diagrams illustrating the calculation of an optimal transport plan for a cell culture while restricting cells to a single type/state (e.g., assuming a single set of dynamics) in accordance with various implementations. FIG. 32A shows an optimal transport plan 3202 of a cell culture from a first timepoint 3204 to a second timepoint 3206. An optimal transport solution for the cell culture calculates cell trajectories including cell motion, division, and death for a transport minimum cost. A set of intermediate cell culture maps 3208 may be generated as part of the optimization of the transport plan.

FIG. 32B shows a graph 3210 of cell dynamics (motion, division, death), shown for illustrative purposes on two axes PC1 and PC2, which for example may represent principal components of a rich description of cell dynamics under various conditions, both local and global. The graph 3210 shows cell dynamics for a single cell type/state (“A”) of the cell culture that the optimal transport plan 3202 is applied to. However, to accomplish the optimal transport plan, significant deviations from the expected cell behavior, indicated by dashed line 3212, may be required.

FIG. 32C shows an alternative graphical plot 3214 showing the energy, or cost, of a transport plan. The optimal transport plan is depicted as the lowest valley “B” in the plot 3214. The actual transport plan (depicted as valley “A”) may require more energy/cost than what would be expected for this cell type.

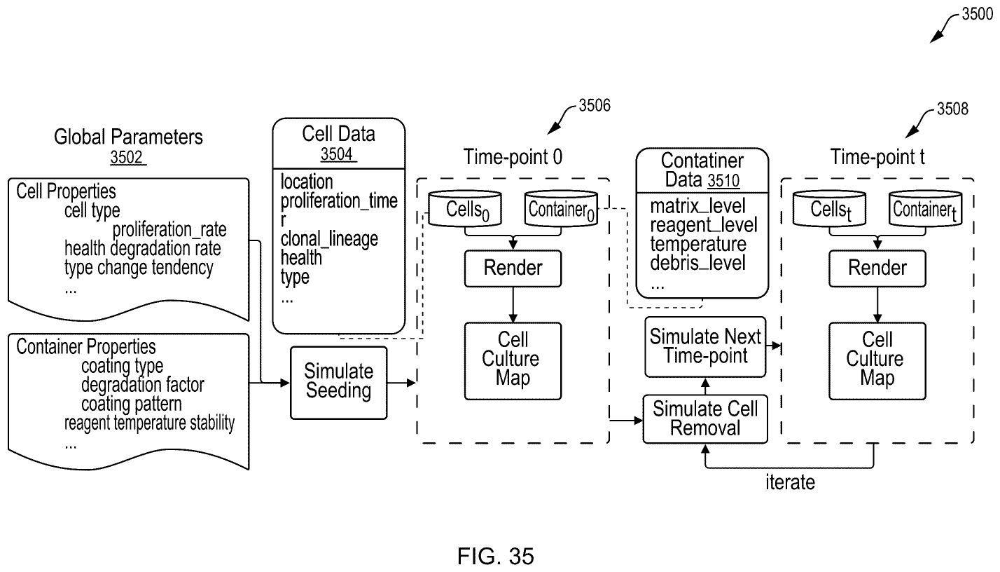

FIGS. 33A-C are diagrams illustrating a solution for the optimal transport challenge depicted in FIGS. 32A-C in accordance with various implementations. The solution may allow the introduction of a second cell type/state, may allow for the possibility of transition from the first cell state into the second cell state, and ultimately also provides a cell map with estimated cell state classifications, based on the observed dynamics of the cell population in the optimal transport solution incorporating multiple cell states.

FIG. 33A shows a revised optimal transport plan 3302 (revised with respect to the optimal transport plan 3202 shown in FIG. 32A). The revised optimal transport plan 3302 has three intermediate spatial states, with two cell states allowed—cell state “A” 3304 and cell state “B” 3306. The subpopulation of cells of the first cell state 3304 expands according to cell type A dynamics, while the subpopulation of cells of the second cell state 3306 expands according to cell type B dynamics. As a result, the revised optimal transport plan 3302 may incur a much lower cost (lower deviation from expected cell behavior) and also provides a spatial classification prediction of the cell populations.

FIG. 33B shows a graph 3308 representing cell dynamics of cells in the revised optimal transport plan 3302, in which cell dynamics have been projected onto two dimensions. Unlike the optimal transport solution depicted in FIGS. 32A-C, during the calculation of the revised optimal transport plan 3302, both cell states A and B are allowed, and transitions 3310 from one state to the other is allowed as part of the solution. This may be constant for the entire time step such that cell states are assumed to be constant, or transitions from one state to another may be allowed in the intermediate estimate states.

FIG. 33C shows an alternative graphical plot 3312 showing the energy, or cost, of a transport plan. The cost or energy on curve 3314 is now minimized by separately optimizing the transport of a first set of cells in cell state A, and a second set of cells in cell state B, with each set of cells following its own cell dynamics (e.g., motion, division, death as a function of local and global conditions). In some implementations, the transition from one state to another may be affected by global or local conditions, as indicated by dashed line 3316. For example, in a colony of iPSCs, a lower barrier to transition to spontaneously differentiated cells may be applied for cells at the edge of a colony, or cells in a very small colony where cells experience stress. In another case, the barrier to a state transition may be lowered due to a change in media constituents meant to initiate cell differentiation.

The process depicted in FIGS. 33A-C may also be reversed. If a map of cell types at a second timepoint is known, the dynamics of these cell types may be used to produce an optimal transport plan from a first timepoint. In such a manner, the cell type regions may be traced back in time. This may be used, for example, when fluorescent labeling or other techniques have been used at a timepoint late in a process (or at a terminal timepoint), to trace the origin of regions classified as different cell types/states spatially. This may be combined with other techniques, such as optimal transport as applied to cell gene expression levels, to predict the evolution of cell state populations.

### Deep Reinforcement Learning Networks for Cell Culture Management

Traditionally, cell culture management has been performed by humans in laboratories, monitoring cell cultures and manually managing the cell cultures, for example by scraping unwanted cells off a cell culture surface or passaging cells between cell culture containers. Automated tools may help laboratory technicians in this task by, for example, collecting images of cells, extracting information from the images that may be used to evaluate cell quality, and removal of cells that have been identified for removal by the technician. However, the technician must still make the final decision on which cells to keep and which to remove. This decision is often a subjective one, and different technicians may make different decisions even when observing the same cell culture.

Efforts have been made to automate the decision-making process performed by technicians in the cell culture process. This would speed up the cell culture process and reduce costs by overcoming reliance on a team of humans. However, these efforts are still in their infancy and often produce poorer results than manual intervention. Thus, there is a need in the art for systems and methods to perform high quality automated cell culture management. In addition, there is a need in the art to perform cell culture management without passaging, as repeated passaging degrades the quality of the cell culture due to cell damage and potential introduction of contaminants.

The systems and methods disclosed herein include an automated method to manage a cell culture (which includes one or more cell clusters) by collecting observation data of a cell culture environment and initiating a series of cell removal operations based on the observation data. These methods replace traditional passaging operations during cell culture processes. A cell culture system as disclosed herein may include an artificial intelligence (AI) engine that performs a series of data processing steps in which microscopic images and other sensor data of the cell culture are inputted on a daily basis. An agent executing within the AI engine may utilize the data to output a cell removal map which includes instructions to an instrument (e.g., a cell removal tool) to execute the physical cell removal operation. The agent may include one or more neural networks called policy models which are trained via a reinforcement learning (RL) technique to decide 1) when to intervene a cell cluster during its proliferation for cell removal, and 2) how to intervene in a cell cluster (e.g., which regions of the cell cluster to be removed or whether the cell cluster needs to be removed entirely). In some implementations, the RL models may be reinforcement learning from human feedback (RLHF) models that utilize human feedback to improve themselves.

The policy models may be trained with a reward framework to keep the cell clusters pure and healthy during their proliferation. For example, for induced pluripotent stem cell (iPSC) cultures, interventions leading to spontaneous differentiation or cell pile-ups (i.e., very dense clusters) generate negative rewards for the agent during training. RL-based techniques utilize large amounts of training data to present many sequences of observation, action, and reward triplets to the agent. For training purposes, a cell culture simulation engine may be developed to initialize the policy models through several in-silico cell culture experiments. The best performing policy models are then adapted to real cell culture behavior through in-vitro cell culture experiment data. The reward mechanism for in-vitro experiments is based on the feedback from expert scientists reviewing the cell culture conditions as well as the outcomes of quality assays run as release criteria for the cell product.

The computing subsystem may include one or more agents that perform the RL methods disclosed herein. The agents may make decisions based on data provided by an AI engine that collects information from the cell culture system. Reinforcement learning is an AI framework to teach an agent how to interact with an environment through carefully designed reward mechanisms. In this framework, the agent usually starts out by naively exploring the environment through random actions and receives rewards after each action or at the end of a series of actions. High rewards reinforce the good choices of the agent, causing better decision making over time. This technique has been successfully applied in many diverse domains recently, such as robotic navigation, stock portfolio management, and game playing.

The systems and methods disclosed herein apply a version of this reinforcement learning technique to train policy models for a cell culture management agent (CCMA). Cell culture management may be framed as a game in which achieving a desired biological outcome at the end of a defined cell culture period establishes a win and any other outcome such as cell death or deviation into an undesired cell state establishes a loss. The implementations disclosed herein also include a training framework to create such agents and a manufacturing system to use such agents.

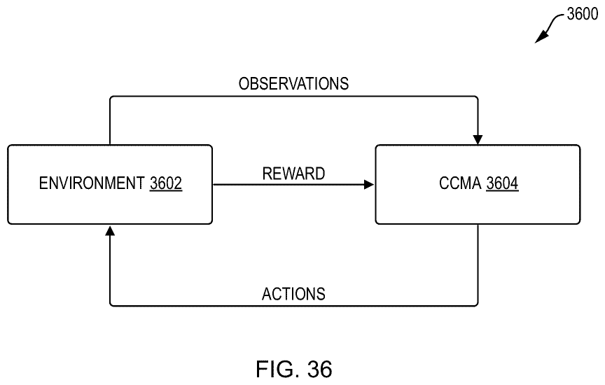

FIG. 34 is a block diagram of a cell culture management system 3400 in accordance with various implementations. The cell culture management system 3400 may be part of a larger cell culture system as disclosed herein. The cell culture management system 3400 may include one or more cell culture containers 3402. A cell culture container 3402 may include one or more cell culture chambers in which adherent cells are cultured on a first surface of the cell culture chamber. The first surface may be optically transparent to allow for optical-based cell imaging and cell removal operations. The cell culture chamber may be sealed during the cell culture process. The first surface may include a laser-activated film or nano-optical coating that is configured to allow for optical cell imaging and precise laser cell removal of cells from the first surface. For example, the film or coating may be configured to absorb laser light within a first wavelength range that targets one or more cells. The film or coating may be configured to convert the absorbed optical energy into mechanical energy to dislodge or otherwise remove the targeted cells from the first surface. The film or coating may also be configured to transmit light within a second wavelength range, this light being used to image the cells without harming them.

The cell culture container 3402 may also contain fluidic components to provide the cell culture with fluid media, to exchange old fluid media with fresh fluid media, and to provide fluidic flows to flush non-adhered cells out of the cell culture chamber. The cell culture container 3402 may also contain various other components to support environmental monitoring or controls, sensors, electronic monitoring and controls, and to couple to robotic arms or other components that may move the cell culture container 3402 around a cell culture system infrastructure. In a non-limiting example, the cell culture container 3402 may be in a closed cassette format that allows for parallel processing of many cell cultures in a shared control system environment. In another example, the cell culture container 3402 may be a multi-well plate.

The cell culture management system 3400 may include a number of instruments 3404 configured to collect data from the cell culture container 3402. The instruments may include, for example, one or more cell imagers 3406, one or more chemical analyzers 3408, and one or more sensors 3410. The cell culture management system 3400 may affect the transition of cells through various bioprocess phases such as reprogramming, maturation, and expansion. During a given cell manufacturing phase, a deployed AI agent may observe the environment of the cell culture container 3402 from observation data 3414 collected from the instruments 3404. The observation data 3414 may include, for example, time-series microscopic image data (which may be multi-channel), nutrient and metabolic data, culture condition data (e.g., pH, 02, temperature), and/or other chemical or condition monitoring data. The observation data 3414 is input to an AI engine 3416, which may have one or more data processing pipelines 3418a-c. The processing pipelines 3418a-c may be configured to process the observation data 3414 to reduce the data to a more abstract input format (such as instances of clones and their attributed measuring pluripotency levels, growth rate, etc.) which are suitable for policy models, and to synchronize multiple data sources to common observation time-points.

The AI engine 3416 also includes a cell culture management agent (CCMA) 3420 that receives processed data from the processing pipelines 3418a-c. The CCMA 3420 may utilize one or more models (e.g., reinforcement learning models) and a mapper 3422 to run inferences on the processed observation data to generate instruction data 3424. In a multiple model scenario, different models may process input from a different input modality, such as culture condition data vs image-timeseries data. Then, a mapper model may ‘join’ the output of these models to an instruction output. In such a multi-modal architecture, multiple ‘heads’ may be used during inference and during a ‘join’ operation. The instruction data 3424 may be used by the cell culture management system 3400 to dynamically manage the cell culture in the cell culture container 3402 in situ. The instruction data 3424 may include a cell removal map 3426 that identifies one or more cells in the cell culture container 3402 may be removed by a cell removal tool 3412. The cell removal map 3426 may be empty if no intervention is necessary (e.g., when cells are in a recovery phase of the cell culture process). The instruction data 3424 may also include time-series image maps showing historical removals applied to the cell culture container 3402. Users of the cell culture management system 3400 may utilize a data viewer 3428 to view all intermediate data products and cell removal maps and provide feedback data 3430 in the form of edits or overwrites to cell removal regions in the cell removal map 3426. Users may be able to ‘view’ the system outputs. Users may also be able to optionally view historical data, for example, for better ‘explainability’ of the actions of the AI engine. Users may also provide feedback data 3430 in the form of regional annotations with associated textual tags to denote various conditions as findings. The cell culture management system 3400 may operate with multiple levels of human intervention. For example, in manual mode a user may have the power to directly manage the cell culture themselves, in a human-in-the-loop mode a user may rely partially on system generated removal instructions, and in an autonomous mode the user may rely entirely on system generated cell removal instructions.

The cell culture management system 3400 may be capable of executing multi-day biological cell culturing processes in which the cell culture is observed on a periodic basis (e.g., daily) and the cell removal map generation is executed at least once per period until some exit criteria are reached. The exit criteria may be, for example, the cells reaching a pre-specified confluence within the cell culture container 3402 and/or a pre-specified number of days passing from the initial cell seeding event. After the exit criteria are met, the cells in the cell culture container 3402 may be transferred to another cell culture container and the CCMA's cell culture management period has ended. The cell manufacturing process may include a plurality of distinct biological phases, and the CCMA 3420 may be configured to execute different and phase-appropriate models depending upon which phase the cells are currently in. For example, in the case of stem cell reprogramming, after starter cell seeding, the CCMA 3420 may run in a reprogramming phase in which all the emerging stem cell clusters are allowed to grow and only spontaneously differentiated cells and non-reprogrammed starter cells are removed. In the case of stem cell culturing, after stem cell seeding into the cell culture container 3402, the CCMA 3420 may run in a management phase in which all stem cell clusters are kept at a pre-determined size and/or density and spontaneously differentiated cells are removed.

In some implementations, data and decisions from in vitro cell culture experiments on the cell culture management system 3400 may also be used to train the models in the CCMA 3420 to improve their performance. For example, in an experiment a seeding event is followed by a series of media change and incubation operations over a period of time for a given cell culture. The cell culture management system 3400 may manage the cell culture process and collect the observation data 3414 on a periodic basis (e.g., daily) while allowing the human experts to monitor the process through the data viewer 3428 on an as-needed basis. Several such experiments, each with different parameters, may be performed on the cell culture management system 3400 to collect the necessary amount of data for training purposes.

The CCMA 3420 may utilize reward mechanisms that are based on the user feedback data 3430, which may be collected in the manual, human-in-the-loop (e.g., RLHF), and/or autonomous modes of operation. Human experts may review each experiment and provide a success or fail assessment based on the biological viability and quality of the managed cell clusters in the cell culture container 3402. This feedback data for an overall experiment is associated, in some embodiments, with each time-point of each cell culture in the cell culture container 3402, thus becoming the reward feedback for each time-point. For failed cell cultures, the human experts may also review previous time-points and provide feedback on the cell removal maps 3426 at the time-points where the CCMA's decisions most likely led to eventual failure outcomes. In alternate or additional implementations, the success or fail assessment of an experiment may come from biological assay outcomes of the samples managed by the CCMA 3420. The assay results may be translated into a pass/fail assessment and such assessment is associated, in some embodiments, with each individual time-point to determine rewards for agent actions at those time-points.

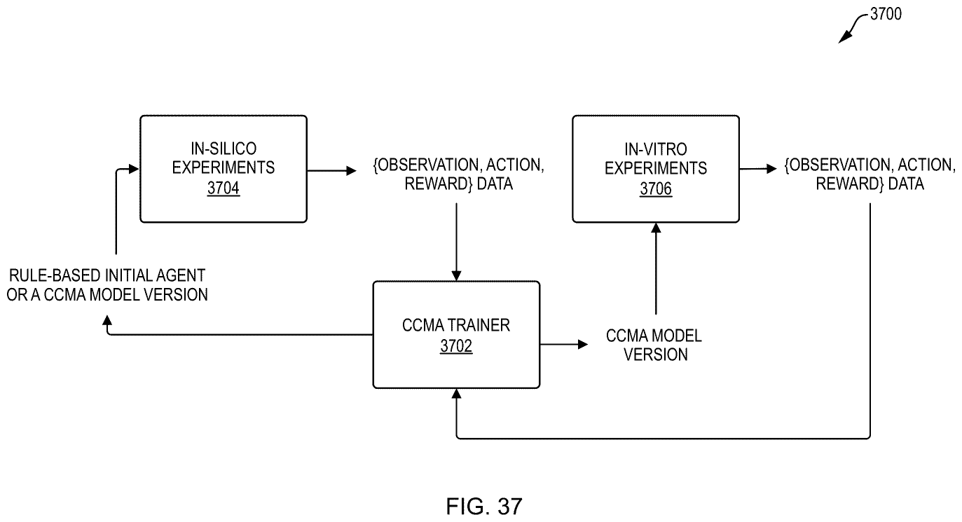

FIG. 35 is a block diagram of a cell culture simulation engine 3500 in accordance with various implementations. The cell culture simulation engine 3500 is configured to mimic cell manufacturing operations on a cell culture system and the data collected from experiments run on the system. The cell culture simulation engine 3500 may be used to train a RL model agent to manage a cell culture. For example, the cell culture simulation engine 3500 may be configured to mimic a seeding event in which a set of cell instances from a particular adherent cell type are initiated in a cell culture container and their locations within the cell culture container are represented as the initial state of the simulated cell culture. The cell culture simulation engine 3500 may store global parameters 3502 that represent global properties of the cell culture container (e.g., laser coating type, reagent type, temperature stability) and each cell in the cell culture container (e.g., cell type, proliferation rate, health degradation rate). The cell culture simulation engine 3500 may also store cell data 3504 for each specific cell, such as location, clonal lineage, type, health, etc.

On a periodic basis (e.g., daily, in simulated time), the cell culture simulation engine 3500 may be configured to mimic cell proliferation, in which new cell instances are added to the simulated cell culture by dividing the existing cells as per their internal timers. Each cell's health, clonal origin, and type are represented and are potentially varied, and the locations of all cells are updated based on the dynamics of the cell culture container as well as the simulated cell population. For example, time-point 0 in block 3506 may represent the state of the cell culture after seeding. The cell culture simulation engine 3500 may render a cell culture map of the simulated cell culture based on the initial properties of the cells and cell culture container (from the global parameters 3502 and the cell data 3504).

The cell culture simulation engine 3500 may mimic the proliferation of the initially seeded cells using the global parameters 3502, the cell data 3504, and cell culture container data 3510 (e.g., reagent level, extracellular matrix level, temperature, debris level) to arrive at a cell culture state at a later time point t, represented in block 3508. The state of the cells and the cell culture container may be updated and then rendered into an updated cell culture map. The cell culture simulation engine 3500 may also be configured to mimic cell removal operations given a cell removal map (instructions) and update the locations of the remaining cells in the cell culture map.

Given a particular population of simulated cells, the cell culture simulation engine 3500 may be configured to simulate observation data (such as images or pH levels) as would be measured by various instruments such as an imager, or culture condition sensors. Specifically, the cell culture simulation engine 3500 may be configured to simulate the 2D spread of cell nuclei in a cell culture container for an adherent cell type after a seeding operation with given settings, subsequent growth characteristics of such seeded cell populations, and after cell removal operations given simulated changing cell culture container conditions such as matrix degradation after cell removal operations. The structure of the simulation, with tracking at the cell level, allows the introduction or evolution of different cell phenotypes or genotypes by way of representing proliferation parameters based on genomic profiles that correspond to a distinct set of cell types, often mixed within cell clusters. For example, extended high density conditions may lead iPSCs to start to differentiate and lose their pluripotency, which in return will change cell proliferation and morphological properties. In another example, karyotypic abnormalities may arise, and change cell mobility or proliferation. This allows training of the CCMA with cell quality feedback. The parameter settings that create a variety of proliferation dynamics for a given set of desired cell types may be manually configured via expert feedback or may be empirically learned from in-vitro cell cultures.

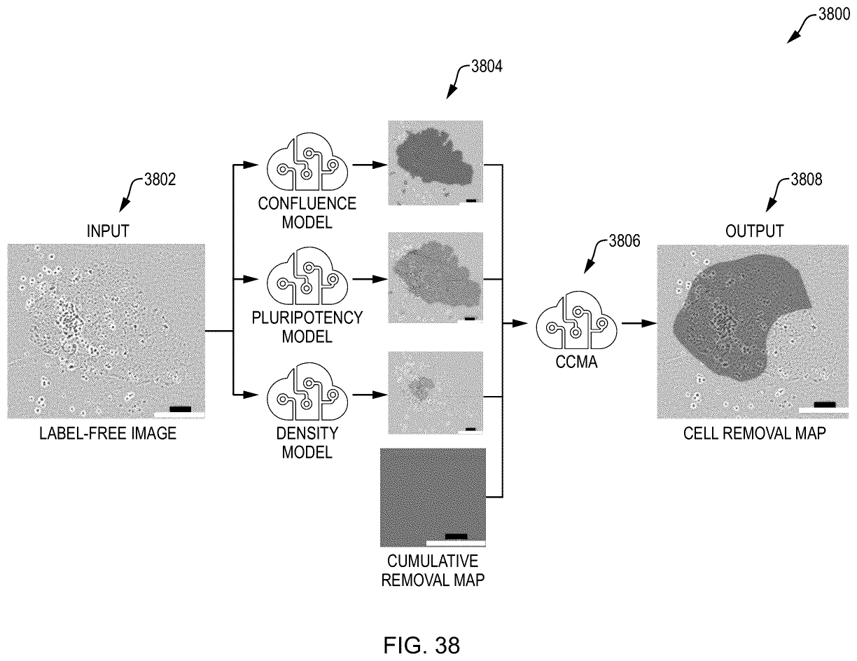

FIG. 36 is a block diagram illustrating a training framework 3600 for a reinforcement learning approach to cell culture management in accordance with various implementations. The training framework 3600 provides reinforcement learning to establish CCMA models that can provide novel cell culture management sequences that achieve highly rewarding cell culture conditions. RL training utilizes sequences of {observation, action, reward}triplets collected from the interactions of the CCMA 3604 with a cell culture environment 3602. “Observation” may include observation data collected from instruments of the cell culture environment (e.g., instruments 3404 in FIG. 34), whether in raw or reduced form, which is inputted to the models of the CCMA 3604. “Action” may include a cell removal map which is outputted from the CCMA 3604. Once the cell removal map is applied, the cell culture environment changes and new observation data is collected after an appropriate period of time. “Reward” may be a scalar value which is generated from this post-action observation data of the cell culture environment to indicate whether the “Action” positively or negatively affected the cell culture according to given criteria. The CCMA 3604 may be trained based on{observation, action, reward}triplets collected from a combination of in-silico and in-vitro experiments.

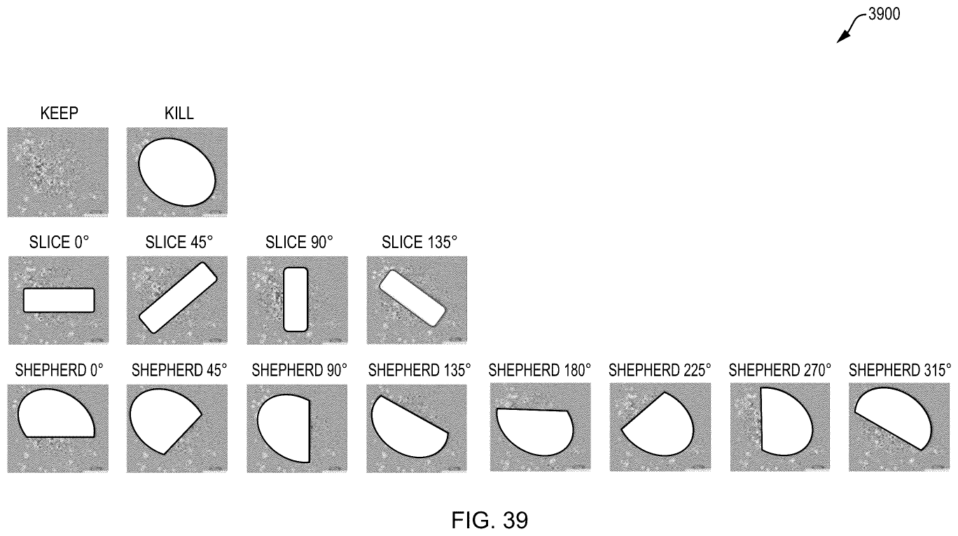

FIG. 37 is a block diagram illustrating the training method 3700 for a reinforcement learning model in accordance with various implementations. A CCMA trainer 3702, for example implementing the framework of FIG. 36, may start with a rules-based initial agent that performs one or more in-silico experiments 3704, for example using the cell culture simulation engine 3500 in FIG. 35. The rules-based initial agent may implement a non-DL based algorithm based on general rules that a human expert managing colonies in a container would follow, for example keeping colonies in concentric circular lanes so that they don't clash with each other nor the container edges. The {observation, action, reward}triplet data from the in-silico experiments 3704 are returned to the CCMA trainer 3702 to train the agent between experiments in a closed loop feedback process. The CCMA trainer 3702 may result in a new CCMA model version which can undergo another in-silico experiment 3704 or an in-vitro experiment 3706 using live cells. The {observation, action, reward}triplet data from the in-vitro experiments 3706 are returned to the CCMA trainer 3702 to update the agent between experiments as well. Note that the in-silico experimentation is cheaper and can be performed longer and as frequently as needed to generate large amounts of {observation, action, reward}data. The reward mechanism measures the performance of any current agent, and the combined in-silico and in-vitro training sessions progressively improve the performance of the agents by having them acquire strategies from in-silico experiments and then fine-tune those strategies on scarcer in-vitro experiment data.

In an example training scheme, using the in-silico experiments 3704 and/or the in-vitro experiments 3706, may mean running each experiment for a period of time with a fixed agent, and recording the outcome. Each experiment is analogous to a playthrough of a game, with a series of steps and data from time-point 0 to a time-point n. In various implementations, the data are recorded in a replay buffer in the format (o0, a0, r0), (o1, a1, r1), . . . , (on, an, rn). A reward value is generated by a reward generator engine for each time-point based on the outcome of the experiment as well as individual time-point events. Given the replay buffer with data from a set of performed experiments, the CCMA trainer 3702 updates the agent. In some implementations, new and past datasets are combined into a composite training set. A new set of experiments are run with a new version of the agent, and the agent is iteratively updated. Models may evolve over several cycles of such training sessions from in-silico and/or in-vitro experiment data.

In an example, a training cycle may begin with a rule-based action generator hand-curated by an expert team to achieve phase-appropriate cell management goals. This rule-based action generator is used in in-silico experiments first and generates an initial training dataset. This dataset is enhanced by some in-vitro experiment data in which human experts correct the rule-based agent's actions and contribute to creation of a larger and more realistic training dataset. Once a decent dataset size, usually on the order of millions of {observation, action, rewards}triplets, is achieved a first training session proceeds to generate a version of CCMA. This strategy is sometimes referred to as imitation learning, in which the agent learns to imitate a human (who would operate based on some domain specific heuristics or rules). Such agents are then used in larger scale in-silico and in-vitro autonomous experiments in which the agent is equipped with some action perturbation as well to enable ‘exploration’ by the agent during that experiment and thus have a chance to potentially devise new strategies and eventually surpass human performance. Action perturbation is a common technique during reinforcement learning training to introduce some noise into the actions of an agent to force it to take possibly a reward reducing or an un-expected action during an episode which may lead to a win in the long run.

The reward generator engine, which may be part of the CCMA trainer 3702, may be used to assign reward values to the experiments managed by the agent. Given data from an episode (o0, a0), (o1, a1), . . . , (on, an) and an experiment outcome such as success or failure, the reward generator engine is configured to assign a reward value to the action of each time-point, specifically creating the data series (o0, a0, r0), (o1, a1, r1), . . . , (on, an, rn) in the replay buffer. For bad outcomes, such as no cell survival at the end of the experiment, the reward engine propagates a negative reward to each time-point. For good outcomes, such as healthy surviving cells with a certain minimum population size, the reward engine propagates a positive reward to each time-point similarly with a decay scheme. Additionally, the reward engine may include a mechanism to examine events logged for each time-point to generate a time-point specific reward to be combined with the value propagated based on the end-of-episode outcome. For example, if there is human feedback available for a given time-point in terms of an edit to the agent's action, then the reward of that time-point is reduced. In another example, if an automated cluster tracking mechanism in the cell culture system logs an event such as managed clusters merging with each other, then the reward of preceding actions are reduced by the reward generator engine.

Different neural network architectures may be used as part of a training framework, depending on input and output formulations. For example, for image-based observational spaces such as cell culture imagery, inference outputs of various CNN-based models that reduce initial raw image to a set of phenotype maps, may be fed as input channels to another CNN to encode task-specific features. These task-specific features can then be fed into a fully connected output layer to generate action probabilities over a discrete action space. In another implementation, raw image observations may be fed into a vision-transformer which connects to a two-headed action generator and a value generator directly in accordance with the actor-critic framework of reinforcement learning.

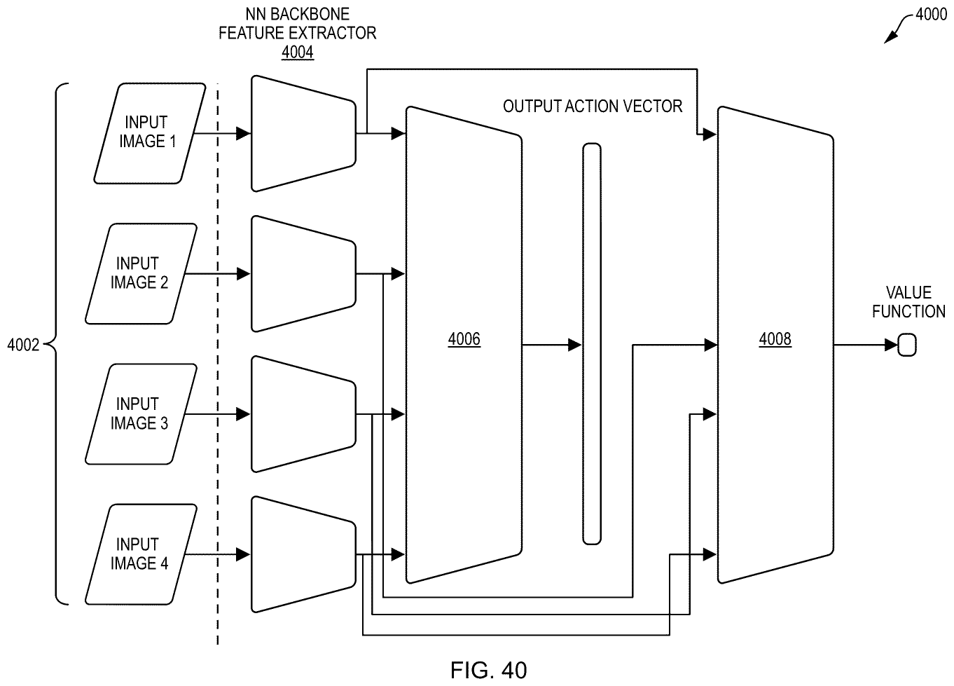

FIG. 38 is a block diagram of a cell culture management system 3800 using an exemplary agent architecture in accordance with various implementations. In the example described herein, the agent may operate at the cell cluster level to decide what the next cell removal action should be from a set of possible actions. The cell culture management system 3800 may take as input 3802 label-free images of a cell culture. The input 3802 may be processed through different models to produce different maps 3804 highlighting different attributes of the cell culture. For example, the maps 3804 may include a cell confluence map generated by a confluence model, a pluripotency map generated by a pluripotency model, a cell density map generated by a density model, and/or a colony health map generated by a colony embedder. In this example, these models are CNN-based segmentation models that reduce inputted label-free images to output images. In this way, a label is applied to one or more region of the image, forming as label map. The models may be configured to act on the input 3802 to detect where the cells are in a cell culture container, what type of cells they are, and what their density is at each grid location in the cell culture container (e.g., a pixel in the inference image).

The maps 3804 may also include a cumulative removal map, which is an image in which each pixel shows how many times a cell removal operation was applied to that physical grid location in the cell culture container over the course of the experiment. This map may provide environment observation information with respect to matrix integrity, so the agent may strategize to stay on ‘good’ (i.e., less damaged) matrix areas to maximize cell stability. The input 3802 may be an image of the full cell culture container and some processing may be done to extract cell clusters based on the inferred confluence map. The maps 3804 used as input to a CCMA 3806 may be cropped and centered around one cluster at a time (which may be termed an “ego-cluster”) so that the CCMA 3806 may choose an action for and with a certain line of sight around it. This cropped area is what the CCMA 3806 ‘observes’ from the environment. If there are other cell clusters in the cell culture container, those clusters may show in this observed area only when they get close enough to the ego-cluster and fall within its line of sight.

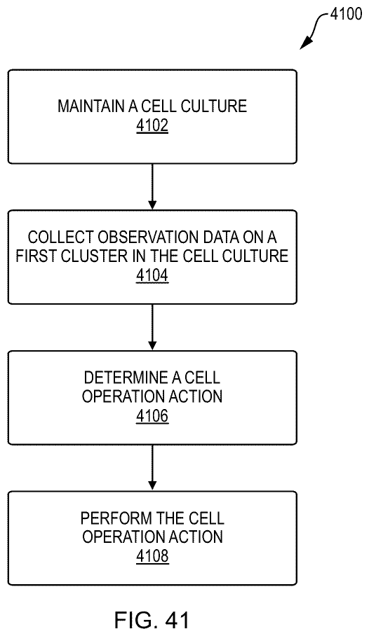

The output of the CCMA 3806 may be a cell removal map 3808 which is generated based on the action output. For example, possible action outputs with respect to cells or cell clusters may include but are not limited to keep, kill, slice with a certain orientation and bite area, and shepherd with a certain orientation and bite area. FIG. 39 illustrates an example action space 3900 for a cell culture management agent in accordance with various implementations. The example action space 3900 includes keep, kill, slice, and shepherd actions, but may also include other actions or operations not illustrated in FIG. 39. In the example shown in FIG. 39, the orientation space is binned into 8 angular intervals for shepherding actions and it is binned into 4 angular intervals for slicing actions. In FIG. 39, the outlined shape in each image illustrates the cell removal map in relation to an example oval shaped cell cluster. Note that the cell removal map may be generated based on a specific cluster's outline where a removal template is scaled to cover the cluster fully and oriented based on the agent's chosen shepherding orientation for example so that all the cells which are not in the ‘mouth’ of the template would be removed completely. The cell cluster outline would be generated from the upstream confluence model output which segments the image into cells vs background and thus enables grouping of neighboring cell pixels as cell cluster objects with an outline. The actions may be implemented by a cell removal tool, such as a laser that may be used to target specific cells and cell clusters and remove them using opto-mechanical means as disclosed herein.

A keep action places no cell removal template onto the cell cluster and hence effectively leaves all cells from that cluster within the cell culture container. A kill action places a template to fully cover the whole cell cluster and thus removes all the cells in that cluster from the cell culture container. A shepherding action removes a chunk of the cells in the cell cluster and affects growth into a particular direction in the cell culture container. Such steering enables the agent to strategize motion within the cell culture container by avoiding clashes onto the edges, avoiding clashes onto other cell clusters that are being managed, and ensuring that cells always grow onto good matrix areas. A slicing action is used to create two new cell clusters from an existing cell cluster and enable the agent to increase the number of cell clusters that are being managed (e.g., to recover from an unwanted merge of clusters). The agent infers one of the actions at each time period for each cell cluster to maintain a number of clusters based on the overall goals of the bioprocess phase and achieve a successful outcome (e.g., survival of all initially selected clusters). In some implementations, the actual cell removal map shape may be different than the action template and may follow exact boundaries of the cell cluster more faithfully.

The complexity of cluster management increases as more clusters are managed within the cell culture container and as the number of days without matrix re-deposition increases. The former is due to increased chances of clashes and the latter is due to the decreased availability of cell culture container surface with intact matrix. Note that cell removal operations damage the matrix coating of the cell culture container and create less ideal conditions for cell growth and thus the agent is taught to avoid entering such potentially damaged areas.

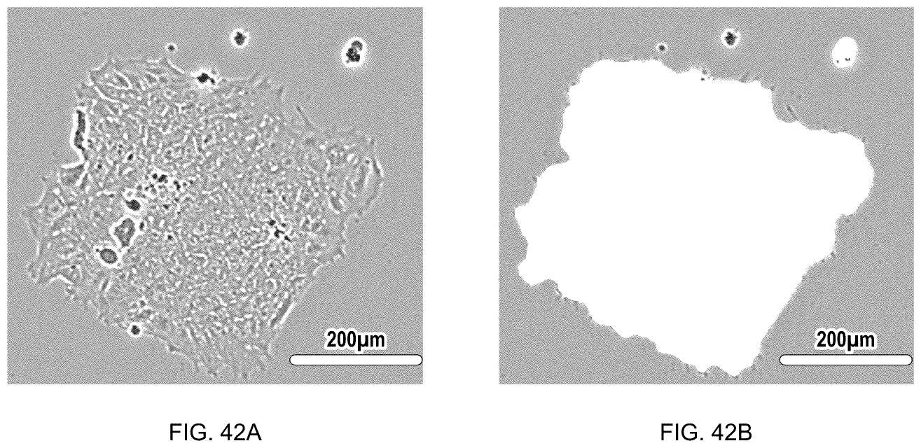

FIG. 40 is a block diagram of an example neural network architecture 4000 used in a cell culture management system in accordance with various implementations. The neural network architecture 4000 may be used by a cluster-level action generator agent as described herein which is applied to each cluster in the container to generate an action for each. This architecture follows the actor-critic paradigm of reinforcement learning and features two heads (outputs) from the neural network: a policy head 4002 and a value head 4004. Specifically, input images 4002 are processed by one or more neural network backbone feature extractors 4004. The output of the extractors 4004 are fed into two full connected networks: an action generator or policy head 4006 to generate an output action vector π(a|s) and also to a value head 4008 to generate a value function V(s). Note that a stands for action and s stands for state, and the input images 4002 (e.g., observation data) are thought to be generated from the current state of the environment and thus represent the state s. In some examples, a temporal memory may be used with one or both of the fully connected networks to account for the sequence of prior steps. In some examples, one or both of the fully connected networks may be a stateless network.

The output action vector or policy function π(a|s) outputs an action for a given input state s (or its observation data). In one example, the action output may be a one-hot vector of size 14 corresponding to 14 possible actions, such as those illustrated in FIG. 39, possibly also including potential angles, and as described herein. The size of the one-hot vector may be handled by changing a size of the template based on the cluster outline. The value function V(s) outputs a scalar value representing the predicted reward at the end of the game for being in the current state s (as observed from the input images 4002). Having an explicit estimation for the states' values allows for policy gradients to be scaled by the amount of expected reward and thus leads to faster convergence during training. The ground-truth for expected reward is usually calculated based on a discounted total reward of all the steps that followed a given current step in a given episode until the end of the game.

Note that given a cluster-level action generator as described herein, the inference pipeline may be executed for each cell cluster in the cell culture container so that the agent generates actions for each cell cluster. Multiple decisions by the agent effectively makes this a multi-agent game even though the same ‘trained’ agent is used to generate actions in a given episode. For a given ego-cluster, the agent takes other cell clusters into account as they fall into its line of sight and thus some collaborative behavior emerges if such collaboration creates good outcomes for the experiments. For example, the agents learn to move clusters into directions where they avoid collisions with each other because in experiments where collisions happen one of the clusters would essentially merge with another one. The collision avoidance behavior is reinforced by carefully designing the reward engine to examine such merge events and propagate back negative rewards to preceding actions.

In some implementations, the neural network architecture 4000 may use several backbones, such as ResNet or a transformer, as its feature generator network and may even transfer weights of models trained on other image-based feature extraction tasks. In some implementations, the neural network architecture 4000 may operate with varying action spaces by increasing the output vector size and assigning new templates to each action category. For example, it can include mouth-size bins (e.g., an area of a cell cluster that remains after excising a portion of the cluster) to represent shepherding actions with varying mouth sizes for each orientation. Some action perturbation may be utilized for in-silico or in-vitro experiments to generate episodes of data where agents experiment with a larger action space and observe outcomes accordingly. Note that as action space increases, the network parameters increase and thus the data required for convergence would increase.

In some implementations, the neural network architecture 4000 may output a full image as the cell removal map directly rather than relying on an intermediate template which gets turned into an image based on the cell cluster outline. Such an output space would give the agent more possibilities to explore the action space, such as creating holes within clusters. In some implementations, the neural network architecture 4000 may use as input a series of images for each of its input channels to represent a historical line of sight around the ego-cluster. Such enlarged observation data space would enable the agent to understand ‘where’ the cluster has been so far within the cell culture container and would enable the agent to learn better strategies for steering. The extractor 4004 then acts on a time-series image input and outputs a single feature vector to be fed into the policy head 4006 and the value head 4008. The backbone network(s), such as ResNet, associated with neural network architecture 4000, may have a modified input channel to have ‘more’ channels. Each additional channel may allow for a different time-point to be input to the backbone network(s). This may allow for the backbone network(s) to receive any number of previous time-points as input, in one shot.

In some implementations, the neural network architecture 4000 may be formalized as a super-agent, with a broader input context and decision-making scope than a single cluster-level agent. With a super-agent, N cell clusters may be managed by a single neural network model. This model would take in N sets of the 4 input images described herein, as well as produce N actions and values. This super-agent may manage all cell clusters in a cell culture container or various groups of cell clusters, possibly arranged by spatial proximity to one another or other characteristics. Super-agent formalizations have the benefit of being mathematically stationary, which removes the blame attribution problem common in multi-agent problem domains. Due to this, as well as the broader input context, it is likely that a higher degree of cell cluster coordination may be achieved with a super-agent. In some examples, the super-agent may be trained to generate a non-action from those output channels that correspond to fully removed/dead clusters at any point before an episode is over.

In some implementations, the neural network architecture 4000 may be equipped with a simulation mechanism (e.g., cell culture simulation engine 3500) during inference time to predict the future outcomes of a plurality of highly probable actions at each step. The agent may then choose an action more optimally based on both the current state of the game and predicted future states resulting from probable actions. For example, the shepherding dynamic of a cell cluster may be simulated by a step size and a shepherding action angle (dθ) to discretize the lookahead search space. A given cell cluster would move by a step size along a given direction/angle in the next time point. All possible positions for each cell cluster may be predicted this way and negative outcomes may be anticipated for the ego-cluster (e.g., a collision with another cluster). Predicted future states may be assigned a scalar value similar to reward values based on the anticipated outcomes of these simulated moves. A Monte Carlo tree search (MCTS) algorithm may be used to evaluate possible future states of the game and pick an optimal move for the ego-cluster from a set of highly probable actions initially inferred by the agent for that time-point. As the depth of the search (lookahead time interval) increases, the complexity of the search tree increases. However, the chance of predicting a favorable outcome for the ego-cluster increases, so there is a trade-off between computation time and having a more effective agent in each episode.

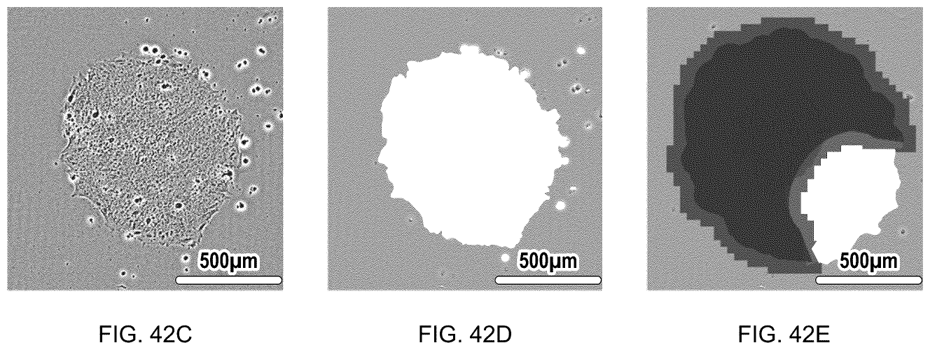

FIG. 41 illustrates a method 4100 for cell culture management in accordance with various implementations. The method 4100 may be performed by a cell culture management system as disclosed herein. In block 4102, a cell culture management system maintains a cell culture in a cell culture container. The cell culture may be, for example, an adherence cell culture (e.g., iPSCs). The cell culture container may be, for example, a closed cassette or multi-well plate. The cell culture container may include a semi-transparent first surface upon which the cell culture is adhered, the first surface configured to allow for optical imaging and optical cell operation action. The cell cluster may include one or more cell clusters.

In block 4104, the cell culture management system may collect observation data on one or more of the cell clusters. The observation data may include time-series images of the cell clusters and information derived from the images (e.g., confluence, density, pluripotency).

In block 4106, a cell culture management agent in the cell culture management system may determine a cell operation action to perform on the one or more cell clusters based on the observation data. The cell culture management agent may utilize reinforcement learning to make the determination. The cell culture management agent may be trained on managing a cell culture through in silico experiments (e.g., using a cell culture simulation engine as described herein) and in vivo experiments.

In block 4108, a cell removal tool in the management system may be configured to perform the determined cell operation action. The cell operation action may include, but is not limited to, removing a cell cluster, removing a region of a cell cluster, shepherding a cell cluster along a surface of the cell culture container, splitting a cell cluster into a plurality of sub-clusters, and keeping a cell cluster as-is. In this manner, the method 800 allows for automated cell culture management using a reinforcement learning scheme, resulting in more efficient, consistent, cheaper, and scalable cell manufacturing.

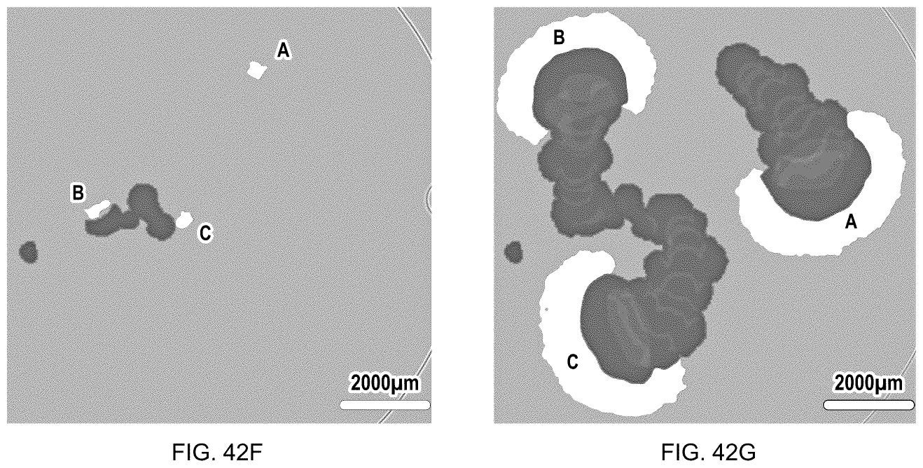

FIGS. 42A-G illustrate machine learning generated (ML-generated) cell removal maps and sub-sequent removal and proliferate steps to expand a cell colony in accordance with various implementations.

FIG. 42A shows an iPSC Colony “A” label-free brightfield image. The image is of the Colony on day 0. FIG. 42B shows an iPSC Colony “A” deep learning-generated live cell map, shown in white. The cell map is generated from an image of the Colony on day 0. FIG. 42C shows another iPSC Colony “A” label-free brightfield image. The image is of the Colony on day 3. FIG. 42D shows another iPSC Colony “A” deep learning-generated live cell map, shown in white. The cell map is generated from an image of the Colony on day 3. FIG. 42E shows an iPSC Colony “A” algorithmically-generated cell removal map, shown in black, to maintain colony size and healthy cell density, and move towards fresh ECM. The cells may have been removed using a cell removal tool, such as a laser-based tool. As examples, cell removal may be achieved using laser-absorbing film absorption and/or laser initiated microbubble cell deletion. The cell removal map is generated from an image of the Colony on day 3. FIG. 42F shows three iPSC Colonies, labeled as “A”, “B”, and “C”, with deep-learning generated live cell maps, in white, and cumulate cell removal maps, in black. The cell maps and cell removal maps are generated from an image of the Colonies on day 0. FIG. 42G shows three iPSC Colonies, labeled as “A”, “B”, and “C”, as is FIG. 42F, with deep-learning generated live cell maps, in white, and cumulate cell removal maps, in black, showing significant expansion of each colony while maintaining healthy cell density. The cell maps and cell removal maps are generated from an image of the Colonies on day 26.

### Systems and Methods for Bioprocess Anomaly Detection

Many cell culture processes require scaling to large numbers of cells. This includes processes using adherent cell cultures, in which cells are in theory observable via microscopy. However for some cell culture processes, for example differentiating and/or expanding cells for regenerative therapies, require on the order of billions of cells per batch, and 1E2-1E4 cm² or even more of growth surface. At the same time, many of these complex processes experience deviations from the desired process, in forms including incorrect cellular differentiation or maturation, poor cell health, unhealthy local cell densities, or other issues.

Typical monitoring practice is to perform microscopy spot checks on the cell culture in just a few locations. Often, only cell confluence (e.g., the proportion of area covered with live cells) is measured as an indicator of progress in the process. Some automated systems, in fact, automate such spot checking which image a very small region of the growth surface. While sampled imaging/spot checks may generate a proxy for overall cell number or state, it does not solve the problem of anomalous cell states/behaviors/arrangements which may result in a loss of manufacturing yield, efficacy, or safety (for example with inclusion of residual pluripotent or multipotent cells in a cell therapy product, and the resulting risk of tumorigenicity or in vivo differentiation to foreign tissues).

Methods for accurately characterizing cell regions for confluence, density, phenotype, dynamic behavior, etc. down to the single-cell level have been described herein, but often require a significant amount of imaging data collection (for example high magnification and spatial resolution, multiple Z focus layers, and/or multiple illumination conditions with accompanying slow acquisition times per unit area, and huge volumes of data to store and transmit) as well as the processing of this data to create accurate maps (for example by the use of pretrained deep learning network architectures that perform image-to-image translation) that require significant computational resources. Thus there is a need in the art to achieve 100% observation coverage of cell cultures and processes to identify potential regions of abnormal or out-of-specification behavior that may reduce yields, efficacy or safety, but doing so in an efficient manner that is scalable to very large areas and long-duration processes.

The systems and methods disclosed herein utilize high-throughput, sparse imaging of cell cultures, in combination with computing methods that identify anomalies, to survey these cell cultures in their entirety with high efficiency and throughput. Anomalies may be detected based on local morphology (as measured by transmitted or reflected light), and/or changes of morphology over time.

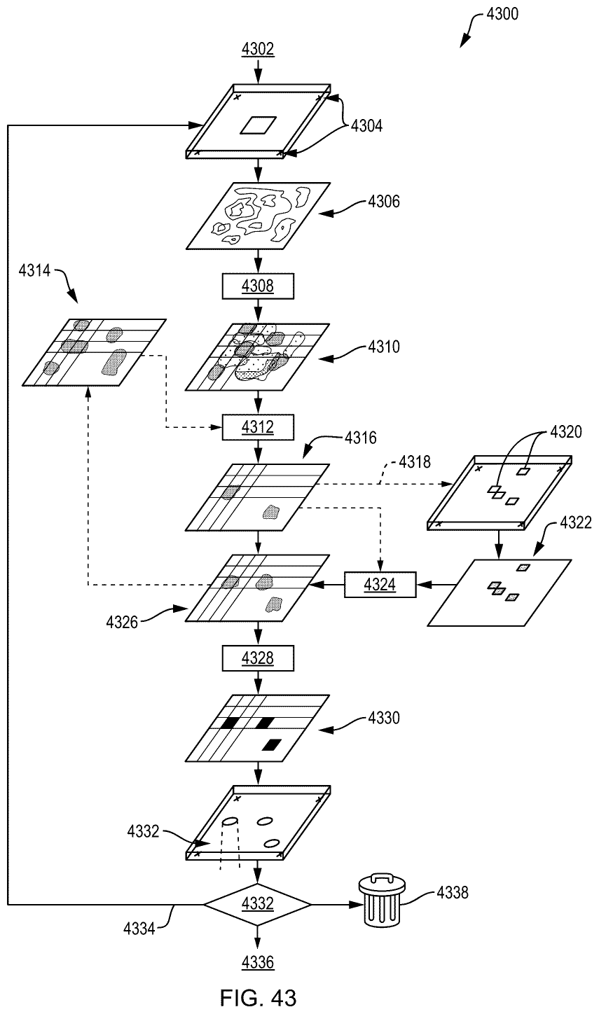

FIG. 43 is a diagram of a cell culture workflow 4300 for detecting anomalies in a cell culture process in accordance with various implementations. A cell sample may be needed into consumable 4302, which may be suitable for adherent or semi-adherent cell cultures and processes. The consumable 4302 may be of various types, including but not limited to closed fluidic cassette formats. The consumable 4302 may include optical registration markings 4304 for the purpose of simplifying alignment between time series images, and coordinating spatial locations between one or more imaging systems as well as, potentially, one or more cell removal tools (CRTs), as have been previously herein. In some implementations, spatial registration may be performed on the basis of cell culture growing internal to the consumable itself.

The consumable 4302 and cell culture within it is imaged by an imaging system to generate images or image composites 4306. This imaging may be done using label-free imaging that provides a very high area throughput to enable imaging of large culture vessels or cassettes, and many such containers that are cultured in parallel. Conventional imaging modalities such as brightfield, phase, quantitative phase, differential interference contrast may be used. In some implementations, as opposed to using a microscope objective, camera systems may be used in conjunction with various controlled illumination. In some implementations, more than one imaging system may be used in parallel to image the containers and cell culture. In some implementations, a system resembling a flatbed scanner (with a substantially 1D photodetector array) may be used in conjunction with controlled illumination. In other implementations, lensless imaging systems may be used with coherent or semi-coherent light sources in order to obtain a “holographic” image of the cell culture. In some implementations using lensed systems, image sensors may be placed in the Fourier plane of the optical system (rather than the image plane) to capture one or more images of the frequency domain representation of the local cell culture.

The images 4306 may be a human-readable image of the cell culture, but may also be a frequency-space representation, or other representation (with one or more 2D planes, if multiple image sensors were used to image each area) that represents the local state of the cell culture and is ultimately interpretable by machine learning models. In some implementations, compressive sensing techniques may be used to minimize acquisition time and the amount of data that needs to be processed. In such cases, the compressive sensing system is configured based on the known information content of the optical signal from the cell culture. For example, if one or more Fourier plane images are recorded by an imaging system, only portions of such an image may be captured. In other cases, a full image at relatively high resolution may be captured, but immediately decomposed into a frequency pyramid format, in which low-resolution images represent larger spatial features, and higher-resolution images represent smaller spatial features. Such an image pyramid may be computed via Gaussian, Laplacian, wavelet or other means, including filter series that are specifically designed for the cell culture features. The purpose of such a pyramid is to allow high-speed surveys of the cell culture via image processing/machine learning algorithms, with closer inspection (via higher-resolution layers) in areas of uncertainty.

A computing system 4308 implements a model which now classifies regions within the cell culture, represented by map 4310 showing embeddings generated by the model from label-free image data. In some implementations, such classifications may correspond to known biological features (live or dead cell presence, cell density, cell morphology, etc.). In other implementations, the model simply categorizes patches with similar features closer to each other in a latent space. This is possible even when data produced by the imaging system is not of the cells themselves, but another representation (Fourier space, holographic, etc.) of the local cell configuration.

Producing a good latent space entails creating a model which represents a given input image, typically with size 256×256 pixels, as a grid of non-overlapping much smaller, typically 16×16 regions (aka patches), and maps each of these regions to a high dimensional vector in a semantically meaningful space conducive to the task of change/anomaly detection. Vision Transformer (ViT) architecture has been shown to perform well in generating such latent spaces with a capacity to transfer well to various other tasks. In one implementation, a ViT model may be trained using contrastive learning techniques which generates positive and negative patch pairs to be mapped to closer or further parts of the latent space respectively by the model. For example, patches may be sampled from ‘spatially’ nearby regions in the same image and augmented in various ways to constitute ‘positive’ pairs (i.e., semantically similar) whereas patches from different images or from spatially distant parts of an image can be sampled as ‘negative’ pairs (i.e., semantically dissimilar). Then the model is trained via contrastive loss to learn to classify the patches as positive versus negative and thus creating a latent space which places semantically similar patches to nearby positions.

In another implementation, masked autoencoding techniques may be used as the self-supervision task in which a significant number of patches in an image are masked for a vision transformer model with encoder and decoder modules to learn to reconstruct the pixel values of the masked patches fully. In this self-supervision framework, the decoder module is only used during training and the encoder module is leveraged for mapping patches to latent space vectors as part of downstream tasks such as anomaly/change detection. Masked autoencoding leads to highly representative latent spaces which capture spatial and semantic content of cell microscopy images well.

Models with ViT architectures have a large number of parameters (e.g., ViT-L/16 has 304 million parameters) and thus the number of images required to build them from scratch is on the order of millions. In scarce data scenarios, publicly available ViTs are fine-tuned to create domain-specific ViTs. In these two implementations, a dataset of cell culture images collected with imagers as part of various experiments may be used to fine-tune publicly available ViTs with a compatible number of input channels. Such a domain-specific ViT may further be specialized to a particular bioprocess or batch or even to a particular patient by further fine-tuning on images collected from experiments of that sub-domain only.

The aforementioned self-supervision techniques lend themselves well to building domain-specific embedding models via smaller classical neural network architectures as well. In one implementation, a regular CNN backbone such as ResNet50 can be trained using contrastive learning on input images of size 256×256. The output of one of the intermediate layers of the CNN, such as layer 2 or layer 3 of ResNet50, may be used to construct one dimensional vectors that map their corresponding receptive fields to vectors in a semantic latent space. Note that in this method each location where a vector is constructed may have overlapping receptive fields in the input image but nevertheless the image would be effectively partitioned into grid patterned regions similar to the ViT method with a unique embedding vector corresponding to each region. One advantage of using a CNN backbone is that they can be trained from scratch from a relatively smaller dataset of domain images such as with the number of images on the order of hundreds of thousands instead of millions to create a domain-specific embedding model.

Given a domain-specific ViT or CNN-based embedding model which produces a latent space vector for a region (or a patch) in an input cell culture image, downstream tasks may be achieved through derived models which use these models as their backbone and which are trained via regular supervision. For example, expert annotations of ‘abnormal’ differentiation patterns for a given differentiation bioprocess may be used to train a classifier module that sits on top of the output of the ViT encoder and that learns to partition the latent space of this domain-specific ViT into normal and abnormal for that bioprocess. When such labeled data is not available, various unsupervised clustering techniques such as k-means or mixture-of-gaussians may be used to build classifiers. For example, given a dataset of images from a particular bioprocess, all regions' vectors may be used to build a mixture of gaussian (MoG) model which represents the ‘expected’ top modes in the dataset. During inference, given a new image, each of its regions' vectors can then be classified as ‘abnormal’ if they are out-of-distribution compared to the MoG model.

In some implementations, the model may be configured to classify regions as either “normal” or “abnormal” based on observations of many cell cultures at the same stage of processing, or simply as “empty,” “same” or “different.” In other implementations the model works only from a single snapshot of the cell culture to classify regions. In other implementations the model works from single timepoint snapshots across a batch of cells, for example a single clone/donor/patient batch that is split across multiple containers.

In other implementations the model works across multiple timepoints such that regions may be seen as changing over time or staying the same in the latent space representation. Such change may be a positive or negative signal, depending on the process. Processing block 4312 represents such (optional) change tracking, depicted in this case as comparison with a prior timepoint mapping 4314 (or multiple timepoints) of the same cell culture, to produce a map that indicates change or rate of change of local features. In some cases, for example during cell expansion or differentiation, change is a positive, and areas that do not show change may be marked for elimination. In other cases, change is a potential negative indicator. For example, nearing the end of a differentiation process, areas with continued proliferation or morphological changes may indicate incorrectly differentiated or undifferentiated cells that pose a tumorigenic risk, and such regions may be marked for elimination. In other cases, such change detection is used in conjunction with condition modulation techniques that have been previously herein. For example, media conditions may be modulated over several timepoints, and the rate of regional cell change is mapped to detect indications of cell state or phenotype.

Given a time-series image dataset which provides roughly equally spaced time-points of a cell culture, e.g., every 12 hours, a generative DL model may be trained via a self-supervision task in which the model predicts the next time-point image given the current time-point image. A generative model trained in such a way would learn both the spatial and the temporal dynamics of cell cultures as they go through various bioprocesses and would generate in minute detail how the culture might look like in the next observation point. In one implementation, the aforementioned encoder-decoder ViT architecture of masked autoencoding framework may be trained where no masking is applied during training but the current time-point image is given to the encoder as the full image and the next time-point image is expected to be generated as the full image by the decoder network. If such a generator model is trained on only ‘normal’ bioprocess images, then it learns the ‘normal’ dynamics of the cell culture. Given such a normal dynamics generator, the next observation point images predicted by the model may be compared to actual observation images and the deviations can be computed via direct image differencing or local correlation measurements.

In other implementations, differencing may be done in other domains, for example in the frequency domain, portion of the frequency domain, or aggregated portions of the frequency domain. In another implementation, the embedding vectors given by the encoder module of the ViT trained via this next-time point image generation task may be used to detect deviations. Note that these embedding vectors would capture temporal as well as spatial dynamics of the cell culture regions in the latent vector space. Specifically, the embedding vectors of corresponding image regions may be compared between current time-point and previous time-point images. If the corresponding vectors deviate too much from each other, e.g., as given by a cosine similarity score threshold, then those regions can be marked as ‘abnormal’ or ‘change’ regions. In another implementation, the ‘expected’ vector modes may be learned from past few time-points' images through unsupervised modeling such as by fitting a MoG. Then the current time-point image's vectors may be compared to this MoG to detect out-of-distribution vectors as ‘abnormal’ or ‘change’. Note that MoGs can be fitted to a group of vectors from past time-point images at image-level or at region-level. The regions may be regular partitions of the image as various sized tiles, or more sophisticated semantic partitioning of the image, e.g., into cell cluster regions tracked over time.

Example implementations of the models described herein are disclosed in Dosovitskiy, A., et al., “An Image is Worth 16×16 Words: Transformers for Image Recognition at Scale,” arXiv preprint arXiv:2010.11929, 2020; Caron, M., et al., “Emerging Properties in Self-Supervised Vision Transformers,” In Proceedings of the IEEE/CVF International Conference on Computer Vision (ICCV), pp. 9650-9660, 2021; Chen, T., et al., “A Simple Framework for Contrastive Learning of Visual Representations,” In Proceedings of the 37th International Conference on Machine Learning (ICML), pp. 1597-1607, 2020; He, K., et al., “Masked Autoencoders Are Scalable Vision Learners,” In Proceedings of the IEEE/CVF Conference on Computer Vision and Pattern Recognition (CVPR), pp. 16000-16009, 2022; and He, K., et al., “Deep Residual Learning for Image Recognition,” In Proceedings of the IEEE Conference on Computer Vision and Pattern Recognition (CVPR), pp. 770-778, 2016, each of which is incorporated by reference in their entireties.

A mapping 4316 (which may or may not incorporate change detection) may be flagged for further inspection (as indicated by arrow 4318) when it indicates regions of uncertainty, or regions with previously unseen properties. In other cases a series of representative regions may be picked in a deliberate manner to inform/refine the models of cell culture. In such a case, the consumable 4302 may be sent to a secondary sensing system that performs measurements on these regions of interest 4320. Such measurements may include further transmitted-light imaging (typically at a higher magnification, resolution, more illumination conditions, more focus planes, or other modes that produce higher-resolution, higher-fidelity, or higher data content than the initial high-throughput imaging), but may also include other modalities including but not limited to spectroscopic measurements, two-photon microscopy, total internal reflection measurements, etc. For example, a series of representative regions may be picked based on rate of change from latent space maps generated via high-throughput lensless imaging. These representative regions may then be measured via Raman spectroscopy, and processed by a pre-trained model that predicts cell state from spectra (which indicated local biochemical composition). Via these measurements, the entire map of the cell culture may now be “recolored” by predicted cell state. The sampled local data 4322 is incorporated into the overall cell culture map by processing module 4324, and a final map 4326 of the cell culture (which optionally incorporates such sampling, and optionally incorporates change measurements) is generated.

A computing module 4328 ingests the map(s) of the cell culture and generates a map of regions to remove from the culture, a process that is performed optionally. A removal map 4330 may be generated in such a case, indicating where cells should be killed, ablated, and/or washed out of the cell culture. In some implementations, material from these regions may be used for assays to measure cell state or phenotype, including but not limited to differentiation or reprogramming state, genomic state, epigenetic state, success or failure of gene editing operations, presence of viral vectors or other payloads, etc.

Optional local cell removal is done in the cell culture container using a CRT as indicated by 4332. For example, cells within a closed fluidic cassette may be killed via a laser tool. Such a laser tool may be used in conjunction with an absorbing layer on the cell growth surface, as described herein, or may directly be absorbed in the target cells and/or surrounding media to kill/remove cells via heating and/or boiling.

The cell culture is evaluated by computing block 4332, based on cell culture maps as well as other potential inputs including but not limited to: time in culture, patient or batch information from previous steps, measurements of spent media, assays of cell material from the cell culture, human observation of the cell culture including but not limited to viewing high-throughput imagery, generated latent space maps, or high-resolution sampled data from the regions of interest 4320, or other inputs. A decision is made from the evaluation to continue cell culture in the container (arrow 4334), go on to the next phase of the process (arrow 4336) which may include harvest of the cells, or introduction of a new set of reagents, or disposal of the container/cell culture (arrow 4338) because it is judged to be not likely to yield good cells.

In some cases, the present implementations may be utilized for cell-based production rather than production of cells themselves. For example, the method may be used for viral vector production, manufacturing or exosomes, processing of one cell type (e.g., immune cells) by another cell type, or other biomanufacturing where the 2D adherent cells perform a useful process rather than being the object of the manufacturing process themselves. In such a case the present implementation is used to maintain the most productive state within the system. In some implementations, computing or processing blocks or modules 4308, 4312, 4324, 4328, and 4332 may performed by a single computing subsystem (e.g., computing subsystem 110 in FIG. 1), or may be performed one or more separate computing subsystems.

### Data-Driven Cell Culture Management

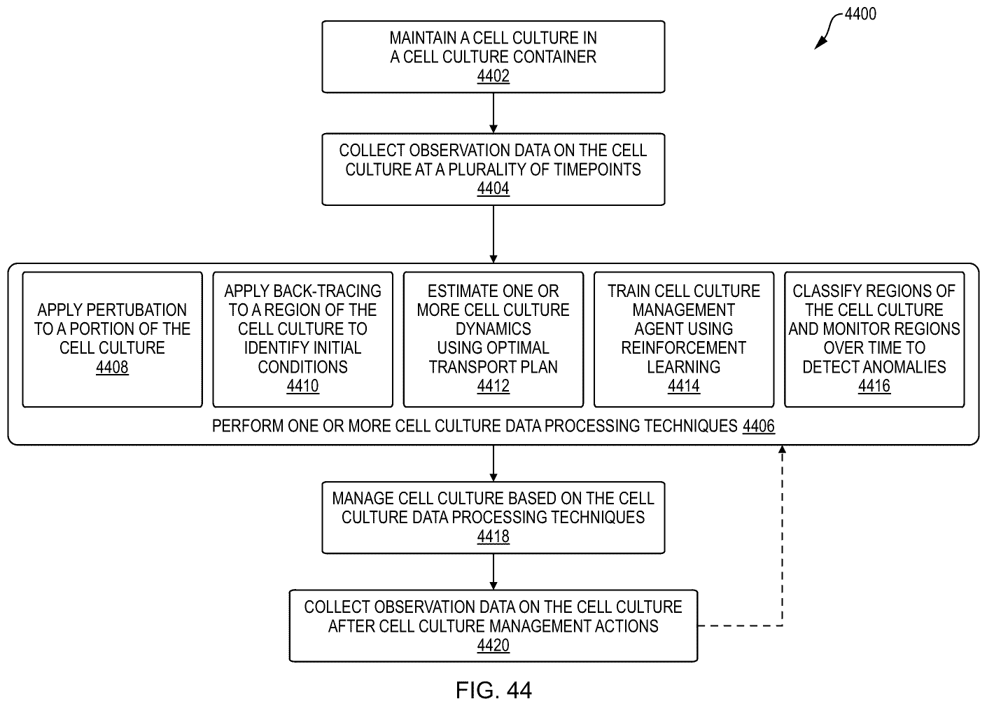

The systems and methods disclosed herein for cell culture measurement and management via modulation, measurement and control of cell culture process trajectories, measurement of cell culture dynamics, management of cell cultures during deep reinforcement learning networks, and detection of phenotype anomalies and changes may be combined with each other in any number of permutations to manage cell culture processes on a cell culture system (e.g., cell culture system 100). FIG. 44 is a flow chart illustrating a method 4400 for measuring and managing a cell culture, and may be performed by a cell culture system.

In block 4402, the cell culture system may maintain a cell culture in a cell culture container (e.g., well plates, closed cassettes). The cell culture may include a plurality of cell colonies/clusters. In some implementations, the cell culture system may include a plurality of cell culture containers and maintain cell cultures in each of them in parallel. The cell culture system may perform a cell culture process on the cell culture, for example but not limited to expansion, reprogramming, differentiation, rejuvenation, regeneration, clonalization, and gene editing.

In block 4404, the cell culture system may collect observation data from the cell culture at a plurality of timepoints. The observation data may include, but is not limited to, imaging data, chemical measurements, or environmental sensor data. For example, the cell culture container may be a closed cassette with at least one transparent surface that allows optical imaging of the cell culture without opening the cell culture container so that the cell culture environment remains sterile. Collecting observation data at a plurality of timepoints allows for the creation of time-series data sets (e.g., time-series images) and enables the tracking of the cell culture and its parameters over time and allows for richer data processing.

In block 4406, the cell culture system may perform one or more cell culture data processing techniques based on the observation data. The cell culture data processing techniques include any of the techniques disclosed herein. For example, in block 4408 the cell culture system may perform one or more perturbations to a portion of the cell culture, as described with reference to FIGS. 3A-6. In another example, in block 4410 the cell culture system may apply back-tracing to a region of the cell culture to identify the initial cell culture conditions of that region, as described with reference to FIGS. 7-25. In another example, in block 4412 the cell culture system may estimate one or more cell culture dynamics using an optimal transport plan, as described with reference to FIGS. 26A-33C. In another example, in block 4414 the cell culture system may train a cell culture management agent using a reinforcement learning model, as described with reference to FIGS. 34-42G. In another example, in block 4416 the cell culture system may classify regions of the cell culture and monitor the regions over time to detect anomalies, as described with reference to FIG. 43.

In block 4418, the cell culture system may manage the cell culture based on the cell culture data processing techniques. Management may include, for example, determining one or more cells, cell colonies, or portions of cell colonies to remove (e.g., using a cell removal tool), determining whether to perform media changes or flushes, determining whether certain environmental parameters of the cell culture should be adjusted to change how the cell culture grows over time, determining whether the cell culture is ready for harvesting, determining whether certain initial conditions of a cell culture should be replicated in future cell culture processes, and updating predictive models that govern management of the cell culture.

In block 4420, the cell culture system may collect additional observation data (e.g., time-series images) after any cell culture management actions taken in block 4420. Additional data processing techniques may be performed on the cell culture, this time incorporating the observation data obtained in block 4420. This allows refinement of the data processing techniques to improve cell culture management (e.g., improvement of various machine learning models, feedback on regions of the cell culture to collect additional data from, etc.). In this manner, the method 4400 enables data-driven cell culture management that leads to quicker and higher quality manufacturing of cells for a variety of purposes.

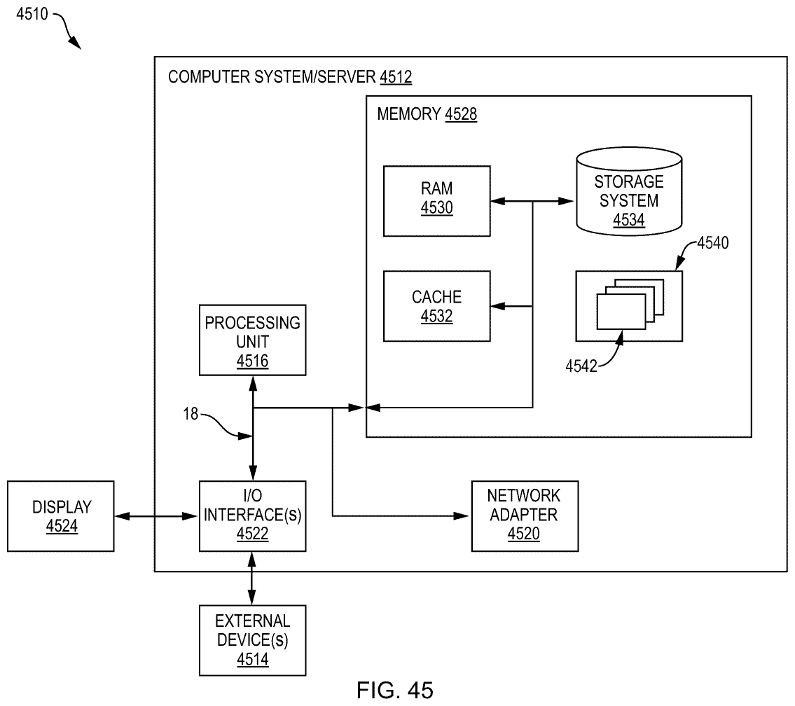

Referring now to FIG. 45, a schematic of an example of a computing node is shown. Computing node 4510 is only one example of a suitable computing node and is not intended to suggest any limitation as to the scope of use or functionality of embodiments described herein. Regardless, computing node 4510 is capable of being implemented and/or performing any of the functionality set forth hereinabove.

In computing node 4510 there is a computer system/server 4512, which is operational with numerous other general purpose or special purpose computing system environments or configurations. Examples of well-known computing systems, environments, and/or configurations that may be suitable for use with computer system/server 4512 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 4512 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 4512 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 45, computer system/server 4512 in computing node 4510 is shown in the form of a general-purpose computing device. The components of computer system/server 4512 may include, but are not limited to, one or more processors or processing units 4516, a system memory 4528, and a bus 4518 that couples various system components including system memory 4528 to processor 4516.

Bus 4518 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 4512 typically includes a variety of computer system readable media. Such media may be any available media that is accessible by computer system/server 4512, and it includes both volatile and non-volatile media, removable and non-removable media.

System memory 4528 can include computer system readable media in the form of volatile memory, such as random access memory (RAM) 4530 and/or cache memory 4532. Computer system/server 4512 may further include other removable/non-removable, volatile/non-volatile computer system storage media. By way of example only, storage system 4534 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 4518 by one or more data media interfaces. As will be further depicted and described below, memory 4528 may include at least one program product having a set (e.g., at least one) of program modules that are configured to carry out the functions of embodiments of the disclosure.

Program/utility 4540, having a set (at least one) of program modules 4542, may be stored in memory 4528 by way of example, and not limitation, as well as an operating system, one or more application programs, other program modules, and program data. Each of the operating system, one or more application programs, other program modules, and program data or some combination thereof, may include an implementation of a networking environment. Program modules 4542 generally carry out the functions and/or methodologies of embodiments as described herein.

Computer system/server 4512 may also communicate with one or more external devices 4514 such as a keyboard, a pointing device, a display 4524, etc.; one or more devices that enable a user to interact with computer system/server 4512; and/or any devices (e.g., network card, modem, etc.) that enable computer system/server 4512 to communicate with one or more other computing devices. Such communication can occur via Input/Output (I/O) interfaces 4522. Still yet, computer system/server 4512 can communicate with one or more networks such as a local area network (LAN), a general wide area network (WAN), and/or a public network (e.g., the Internet) via network adapter 4520. As depicted, network adapter 4520 communicates with the other components of computer system/server 4512 via bus 4518. It should be understood that although not shown, other hardware and/or software components could be used in conjunction with computer system/server 4512. Examples, include, but are not limited to: microcode, device drivers, redundant processing units, external disk drive arrays, RAID systems, tape drives, and data archival storage systems, etc.

In various embodiments, the processors or processing units 4516 may implement the techniques described herein in conjunction with a camera and/or camera system (not shown) that may capture images of various biological materials, such as a cell culture, and/or a data store to store data, such as observation data. The processors or processing units 4516, the camera and/or camera system, and/or the data store may be integrated into a single system, such as an imaging system or device.

The present disclosure may be embodied as a system, a method, and/or a computer program product. The computer program product may include a computer readable storage medium (or media) having computer readable program instructions thereon for causing a processor to carry out aspects of the present disclosure.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise, and encompass “at least one.” Any reference to “or” herein is intended to encompass “and/or” unless otherwise stated.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. As used herein in the specification and in the claims, “or” should be understood to have the same meaning as “and/or” as defined above.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “flexible” as used herein refers to an object or material that is able to be bent or compressed without cracking or breaking. The term “semi-flexible” as used herein refers to an object or material that has a portion thereof that is able to be bent or compressed without cracking or breaking.

As used in any implementation herein, a “circuit” or “circuitry” may include, for example, singly or in any combination, hardwired circuitry, programmable circuitry, state machine circuitry, and/or firmware that stores instructions executed by programmable circuitry. An “integrated circuit” may be a digital, analog or mixed-signal semiconductor device and/or microelectronic device, such as, for example, but not limited to, a semiconductor integrated circuit chip.

The term “coupled” as used herein refers to any connection, coupling, link or the like by which signals carried by one system element are imparted to the “coupled” element. Such “coupled” devices, or signals and devices, are not necessarily directly connected to one another and may be separated by intermediate components or devices that may manipulate or modify such signals.

Likewise, the terms “connected” or “coupled” as used herein in regard to mechanical or physical connections or couplings is a relative term and does not require a direct physical connection.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems.

It will be appreciated by those skilled in the art that any block diagrams herein represent conceptual views of illustrative circuitry embodying the principles of the disclosure. Similarly, it will be appreciated that any flow charts, flow diagrams, state transition diagrams, pseudocode, and the like represent various processes which may be substantially represented in computer readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

While various implementations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the implementations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific inventive implementations described herein. It is, therefore, to be understood that the foregoing implementations are presented by way of example only and that, within the scope of the appended claims and equivalents thereto, implementations may be practiced otherwise than as specifically described and claimed. In addition, any combination of two or more such features, systems, aspects, articles, materials, kits, and/or methods, if such features, systems, aspects, articles, materials, kits, and/or methods are not mutually inconsistent, is included within the inventive scope of the present disclosure. Particularly, any element of the disclosure and any aspect thereof may be combined, in any order and any combination, with any other element of the disclosure and any aspect thereof.

The above-described implementations can be implemented in any of numerous ways. For example, the implementations may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device. Also, a computer may have one or more input and output devices. These devices can be used, among other things, to present a user interface. Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

Implementations of the methods described herein may be implemented using a processor and/or other programmable device. To that end, the methods described herein may be implemented on a tangible, non-transitory computer readable medium having instructions stored thereon that when executed by one or more processors perform the methods. The computer readable medium may include any type of tangible medium, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of media suitable for storing electronic instructions.

The terms “program” or “software” are used herein in a generic sense to refer to any type of computer code or set of computer-executable instructions that can be employed to program a computer or other processor to implement various aspects of implementations as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various implementations. Also, data structures may be stored in computer-readable media in any suitable form.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

### Non-Transitory Computer Readable Storage Medium

In some implementations, the platforms, systems, media, and methods disclosed herein include one or more non-transitory computer readable storage media encoded with a program including instructions executable by the operating system of an optionally networked computing device. In further implementations, a computer readable storage medium is a tangible component of a computing device. In still further implementations, a computer readable storage medium is optionally removable from a computing device. In some implementations, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some implementations, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some implementations, a computer program comprises one sequence of instructions. In some implementations, a computer program comprises a plurality of sequences of instructions. In some implementations, a computer program is provided from one location. In other implementations, a computer program is provided from a plurality of locations. In various implementations, a computer program includes one or more software modules. In various implementations, a computer program includes, in part or in whole, one or more web applications, one or more mobile applications, one or more standalone applications, one or more web browser plug-ins, extensions, add-ins, or add-ons, or combinations thereof.

### Software Modules

In some implementations, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various implementations, a software module comprises a file, a section of code, a programming object, a programming structure, or combinations thereof. In further various implementations, a software module comprises a plurality of files, a plurality of sections of code, a plurality of programming objects, a plurality of programming structures, or combinations thereof. In various implementations, the one or more software modules comprise, by way of non-limiting examples, a web application, a mobile application, and a standalone application. In some implementations, software modules are in one computer program or application. In other implementations, software modules are in more than one computer program or application. In some implementations, software modules are hosted on one machine. In other implementations, software modules are hosted on more than one machine. In further implementations, software modules are hosted on a distributed computing platform such as a cloud computing platform. In some implementations, software modules are hosted on one or more machines in one location. In other implementations, software modules are hosted on one or more machines in more than one location.

### Databases

In some implementations, the platforms, systems, media, and methods disclosed herein include one or more databases/data stores, or use of the same. In view of the disclosure provided herein, those of skill in the art will recognize that many databases are suitable for storage and retrieval of image data, cell types, attribute categories, labels, assay data, or any combination thereof. In various implementations, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some implementations, a database is internet-based. In further implementations, a database is web-based. In still further implementations, a database is cloud computing-based. In a particular implementation, a database is a distributed database. In other implementations, a database is based on one or more local computer storage devices.

What is claimed is:

1. A method of managing a cell culture process, comprising:
maintaining a cell culture in a cell culture chamber, the cell culture undergoing a cell culture process;
measuring one or more properties of the cell culture;
applying one or more perturbations to all or a portion of the cell culture;
measuring the one or more properties of the cell culture after applying the one or more perturbations;
comparing the one or more properties before and after the one or more perturbations; and
managing the cell culture process based on the comparison.

2. The method of claim 1, wherein managing the cell culture process comprises predicting a future state of the cell culture based on the one or more properties before and after the one or more perturbations and the comparison.

3. The method of claim 1, wherein measuring the one or more properties comprises acquiring images of the cell culture.

4. The method of claim 3, wherein the images comprise time-series images.

5. The method of claim 1, wherein at least one of the one or more perturbations is applied spatially.

6. The method of claim 1, wherein at least one of the one or more perturbations is applied temporally.

7. The method of claim 1, wherein the cell culture chamber comprises a closed fluidic cell culture chamber.

8. The method of claim 7, wherein the cell culture is adhered to a first surface of the closed fluidic cell culture chamber.

9. The method of claim 8, wherein the first surface is transparent and enables optical imaging and optical cell removal of the cell culture.

10. The method of claim 1, wherein measuring the one or more properties comprises generating one or more cell maps.

11. The method of claim 10, wherein comparing the one or more properties comprises generating one or more differential maps.

12. The method of claim 1, wherein managing the cell culture comprises at least one of selectively removing one or more cells from the cell culture using a cell removal tool, applying one or more additional perturbations to the cell culture, harvesting the all or a portion of the cell culture for downstream processing, or terminating the cell culture process.

13. The method of claim 1, wherein the one or more perturbations are applied to a test portion of the cell culture spatially separated from the rest of the cell culture.

14. The method of claim 1, further comprising:
capturing images of the cell culture at a plurality of timepoints throughout the cell culture process, the plurality of timepoints comprising an initial timepoint before application of the one or more perturbations and a final timepoint after application of the one or more perturbations;
generating a map of the cell culture at the final timepoint, wherein the map identifies one or more regions of the cell culture having a target cell type;
back-tracing the one or more regions to earlier timepoints;
determining a first perturbation in the one or more perturbations that results in the one or more regions having the target cell type based on the back-tracing; and
associating the first perturbation with the target cell type in a predictive model.

15. The method of claim 1, wherein the cell culture process comprises one of reprogramming, expansion, differentiation, and rejuvenation.

16. The method of claim 15, wherein the map is a spatial trajectory map, an -omic trajectory map, or a combination of both.

17. The method of claim 15, wherein the method is iteratively repeated to improve the predictive model.

18. The method of claim 15, further comprising:
applying the predictive model to a future cell culture process.

19-65. (canceled)

66. A computer program product for managing a cell culture process, the computer program product comprising a computer readable storage medium having program instructions embodied therewith, the program instructions executable by a processor to cause the processor to perform a method comprising:
maintaining a cell culture in a cell culture chamber, the cell culture undergoing a cell culture process;
measuring one or more properties of the cell culture;
applying one or more perturbations to all or a portion of the cell culture;
measuring the one or more properties of the cell culture after applying the one or more perturbations;
comparing the one or more properties before and after the one or more perturbations; and
managing the cell culture process based on the comparison.

67. An imaging system for managing a cell culture process, comprising:
a camera configured to capture at least one digital image of the cell culture; and
a processor configured to receive the at least one digital image from the camera and perform thereon a method comprising:
maintaining a cell culture in a cell culture chamber, the cell culture undergoing a cell culture process;
measuring one or more properties of the cell culture;
applying one or more perturbations to all or a portion of the cell culture;
measuring the one or more properties of the cell culture after applying the one or more perturbations;
comparing the one or more properties before and after the one or more perturbations; and
managing the cell culture process based on the comparison.
